SEQUENCE LISTING (23 sequences)

SEQ ID NO: 1 - DNA, 20 bp, Artificial Sequence
  source: /note=“Description of Artificial Sequence: Synthetic primer”
gtcatttgcaccttcctcct

SEQ ID NO: 2 - DNA, 20 bp, Artificial Sequence
  source: /note=“Description of Artificial Sequence: Synthetic primer”
tggatcaaatgccaggacag

SEQ ID NO: 3 - DNA, 23 bp, Artificial Sequence
  source: /note=“Description of Artificial Sequence: Synthetic primer”
tcctgagaaagagaaggacattg

SEQ ID NO: 4 - DNA, 21 bp, Artificial Sequence
  source: /note=“Description of Artificial Sequence: Synthetic primer”
ctgtgggctcctgtagaaatc

SEQ ID NO: 5-6 - skipped (2 entries)

SEQ ID NO: 7 - DNA, 15 bp, Artificial Sequence
  source: /note=“Description of Artificial Sequence: Synthetic probe”
tggcaacccttgaga

SEQ ID NO: 8 - DNA, 15 bp, Artificial Sequence
  source: /note=“Description of Artificial Sequence: Synthetic probe”
tcgtggtggcaaccc

SEQ ID NO: 9 - DNA, 439 bp, Homo sapiens
gggatgctgcactgtatcagtccctgcccactctggcccgggccctggcacagtacctggtggtggtctccaaactgcccagtcatttgcaccttcctcctgagaaagagaaggacattgtgaaattcgtggtggcaacccttgagaggcaagccctggtgctgtgggagccccaaggaagagcctctggcctggtggccacgtagcccaggagagatttctacaggagcccacagcgctgaaggagagagaggcagcagagccctgtcctggcatttgatccatgagcagatcccgctgagtctggatctccaggcagggctggactgctgctgcctggccctgcagctgcctggcctctggagcgtggtctcctccacagagtttgtgacccacgcctgctccctcatctactgtgtgcacttcatcctggaggccg

SEQ ID NO: 10 - DNA, 22 bp, Artificial Sequence
  source: /note=“Description of Artificial Sequence: Synthetic primer”
gagactcatccagtaccatcag

SEQ ID NO: 11 - DNA, 22 bp, Artificial Sequence
  source: /note=“Description of Artificial Sequence: Synthetic primer”
gatgtcagctatctgtcgagac

SEQ ID NO: 12 - DNA, 19 bp, Artificial Sequence
  source: /note=“Description of Artificial Sequence: Synthetic probe”
ttgtcttcattctccttgt

SEQ ID NO: 13 - DNA, 24 bp, Artificial Sequence
  source: /note=“Description of Artificial Sequence: Synthetic primer”
gtagaacttcagaccctaatcctg

SEQ ID NO: 14 - DNA, 19 bp, Artificial Sequence
  source: /note=“Description of Artificial Sequence: Synthetic primer”
caccactctggcttcacaa

SEQ ID NO: 15 - DNA, 15 bp, Artificial Sequence
  source: /note=“Description of Artificial Sequence: Synthetic probe”
gagtcagtgattgtt

SEQ ID NO: 16 - DNA, 24 bp, Artificial Sequence
  source: /note=“Description of Artificial Sequence: Synthetic primer”
tcactgaagagtaccagaaaagtc

SEQ ID NO: 17 - DNA, 21 bp, Artificial Sequence
  source: /note=“Description of Artificial Sequence: Synthetic primer”
ttttattccccagcactctcg

SEQ ID NO: 18 - DNA, 17 bp, Artificial Sequence
  source: /note=“Description of Artificial Sequence: Synthetic probe”
atacgtggttggagagc

SEQ ID NO: 19 - DNA, 105 bp, Homo sapiens
  misc_feature (1)..(105): /note=“This sequence may encompass 6-35 ′CAG′ repeating units”
cagcagcagcagcagcagcagcagcagcagcagcagcagcagcagcagcagcagcagcagcagcagcagcagcagcagcagcagcagcagcagcagcagcagcag

SEQ ID NO: 20 - DNA, 117 bp, Homo sapiens
  misc_feature (1)..(117): /note=“This sequence may encompass 36-39 ′CAG′ repeating units”
cagcagcagcagcagcagcagcagcagcagcagcagcagcagcagcagcagcagcagcagcagcagcagcagcagcagcagcagcagcagcagcagcagcagcagcagcagcagcag

SEQ ID NO: 21 - DNA, 120 bp, Homo sapiens
  source: /note=“See specification as filed for detailed description of substitutions and preferred embodiments”
cagcagcagcagcagcagcagcagcagcagcagcagcagcagcagcagcagcagcagcagcagcagcagcagcagcagcagcagcagcagcagcagcagcagcagcagcagcagcagcag

SEQ ID NO: 22 - DNA, 108 bp, Homo sapiens
  source: /note=“See specification as filed for detailed description of substitutions and preferred embodiments”
cagcagcagcagcagcagcagcagcagcagcagcagcagcagcagcagcagcagcagcagcagcagcagcagcagcagcagcagcagcagcagcagcagcagcagcag

SEQ ID NO: 23 - DNA, 291 bp, Homo sapiens
cagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcag

(19) United States
(12) Patent Application Publication
(10) Pub. No.: US 2024/0216369 A1
(43) Pub. Date: Jul. 4, 2024

(54) THE USE OF A SPLICING MODULATOR FOR A TREATMENT SLOWING PROGRESSION OF HUNTINGTON'S DISEASE

(71) Applicant: Novartis AG, Basel (CH)

(72) Inventors: Martin BEIBEL, Freiburg (DE); Beth BOROWSKY, Flemington, NJ (US); Jang-Ho CHA, Arlington, MA (US); Thomas FALLER, Reinach (CH); Baltazar GOMEZ-MANCILLA, Rosenau (FR); Caroline GUBSER KELLER, Basel (CH); Marc LAISNEY, Mulhouse (FR); Wen LIN, Millburn, NJ (US); Nicole RENAUD, Somerville, MA (US); Rajeev SIVASANKRAN, Acton, MA (US)

(21) Appl. No.: 17/755,435

(22) PCT Filed: Oct. 30, 2020
(86) PCT No.: PCT/IB20/60210

§ 371 Date: Apr. 28, 2022

Related U.S. Application Data
(60) Provisional application No. 62/929,582, filed on Nov. 1, 2019.
(60) Provisional application No. 62/930,776, filed on Nov. 5, 2019.
(60) Provisional application No. 62/949,698, filed on Dec. 18, 2019.
(60) Provisional application No. 62/963,836, filed on Jan. 21, 2020.
(60) Provisional application No. 63/027,124, filed on May 19, 2020.

(51) Int. Cl. A61K 31/501; A61K 9/00; A61K 9/08; A61K 45/06; A61K 47/40; A61P 25/28

(52) U.S. Cl. CPC A61K 31/501; A61K 9/0053; A61K 9/08; A61K 45/06; A61K 47/40; A61P 25/28

(57) ABSTRACT

Use of a splicing modulator for a treatment slowing progression of Huntington's disease.

### SEQUENCE LISTING

The instant application contains a Sequence Listing which has been submitted electronically in ASCII format and is hereby incorporated by reference in its entirety. Said ASCII copy, created on Oct. 22, 2020, is named PAT058724-WO-PCT_SL.txt and is 6,792 bytes in size.

### FIELD OF THE INVENTION

The invention relates to the use of a splicing modulator for a treatment slowing progression of Huntington's disease.

### BACKGROUND OF THE INVENTION

Huntington's disease (HD) is a hereditary, neurodegenerative and progressive disorder, which has a prevalence of about 5 in 100,000 worldwide. It is caused by CAG repeat expansions in the huntingtin gene (i.e. gene encoding the protein huntingtin) and it is characterized by motor, cognitive, psychiatric and functional capacity decline. The CAG trinucleotide repeat expansion results in a mutant huntingtin protein (mHTT), which is associated with neural dysfunction and ultimately death.

The number of CAG repeats in the HTT gene ranges from 6 to 35 (SEQ ID NO: 19) in healthy individuals. Disease penetrance is seen to be reduced for individuals carrying 36 to 39 CAG repeats (SEQ ID NO: 20), however those with 40 or more CAG repeats (SEQ ID NO: 21) are almost certain to develop the disease. As described in European Journal of Neurology, 2017, 24-34, clinical diagnosis of HD is based on:
confirmed family history or positive genetic test (i.e. confirmation of CAG repeat expansion≥36 (SEQ ID NO: 22)); and
onset of motor disturbance as defined by the Unified Huntington's Disease Rating Scale (UHDRS) total motor score (TMS) diagnostic confidence score (DCS), which ranges from 0 (no motor abnormalities suggestive of HD) to 4 (motor abnormalities≥99% likely to be due to HD), wherein a score of 4 defines “motor onset” or “manifest” HD.

Typically, age of onset (i.e. once the DCS reaches 4) ranges between 30 to 50 years and average duration of survival after clinical diagnosis is 15 to 20 years. Currently, after onset, it is “function” (i.e. assessment of functional capacities), rather than motor signs, which determines disease stage (e.g. in Neurology, 1979, 29, 1-3 or in Neurology, 1981, 31, 1333-1335). The Total Functional Capacity (TFC) scale (e.g. in Movement Disorders, 1996, 11, 136-142) is a component of the UHDRS and ranges from 0 (fully dependent for all care) to 13 (fully independent) the level of independence of a person with HD. This scale assesses functional status of a HD patient in terms of ability to work, handle household finances, manage domestic chores, perform activities of daily living, and level of care needed. Based on the UHDRS total functional capacity (TFC), HD is divided into stages 1 to 5 of disease progression. The categorization of HD, based on TFC score (also referred to as Shoulson and Fahn stages), are also described as early stage of HD (corresponding to stages 1 or 2, based on TFC score), moderate stage or mid stage HD (corresponding to stage 3, based on TFC score) and advanced stage or late stage HD (corresponding to stage 4 or 5, based on TFC score).

At present, only symptomatic treatments are available. Thus, to date, there is no therapy available to slow the progression of HD. Accordingly, there is a need to find disease-modifying therapies for HD (i.e. therapeutic options that can slow disease progression).

### SUMMARY OF THE INVENTION

The invention relates to the use of a splicing modulator, for example, as defined herein:
in a treatment slowing progression of Huntington's disease;
in a treatment slowing progression of Huntington's disease by producing an in-frame stop codon between exons 49 and 50 in the HTT mRNA;
in the treatment of Huntington's disease as a disease-modifying therapy;
in a treatment slowing the decline of motor function associated with Huntington's disease;
in a treatment slowing cognitive decline associated with Huntington's disease;
in a treatment slowing psychiatric decline associated with Huntington's disease;
in a treatment slowing the decline of functional capacity associated with Huntington's disease; or,
in a treatment slowing the progression of Huntington's disease pathophysiology.

### BRIEF DESCRIPTION OF DRAWINGS

FIG. 1. RNA-seq analysis of human fibroblast line treated with branaplam. FC: fold change. RPKM: Reads per kilo base per million mapped reads.

FIG. 2. 2a, 2b, 2c: In vitro modulation of HTT transcript and protein.

FIG. 3. A single, oral dose of branaplam elevates novel-exon-containing brain HTT transcript levels in the BacHD mouse model. P values: (Vehicle 8 hrs vs 50 mg/kg, 8 hrs) ** P=0.0034, (Vehicle 24 hrs vs 50 mg/kg, 24 hrs) **** P<0.0001, (Vehicle 24 hrs vs 50 mg/kg, 48 hrs) ** P=0.0020.

FIG. 4. A single, oral dose of branaplam lowers total brain HTT transcript levels in the BacHD mouse model.

FIG. 5. A single, oral dose of branaplam elevates novel-exon-containing blood HTT transcript levels in the BacHD mouse model.

FIG. 6. A single, oral dose of branaplam lowers total blood HTT transcript levels in the BacHD mouse model.

FIG. 7. Repeat oral doses of branaplam in the BacHD mouse model lowers mutant Huntingtin protein in the striatum. P values: (Vehicle vs 12 mg/kg, 24 hrs) ** P=0.048, (Vehicle vs 24 mg/kg, 72 hrs) *** P=0.0003, (Vehicle vs 24 mg/kg, 72 hrs) **** P<0.0001.

FIG. 8. Repeat oral doses of branaplam in the BacHD mouse model lowers mutant HTT protein in the cortex. P values—(Vehicle vs 12 mg/kg, 24 hrs) ** P=, (Vehicle vs 24 mg/kg, 72 hrs) ***P, (Vehicle vs 24 mg/kg, 72 hrs) **** P<0.0001.

FIG. 9. Repeat oral doses of branaplam in the BacHD mouse model lowers mutant HTT protein in the liver.

FIG. 10. Repeat oral doses of branaplam in the BacHD mouse model lowers total HTT transcript in blood.

FIG. 11. Repeat oral doses of branaplam in the BacHD mouse model lowers HTT mutant HTT protein in the CSF.

FIG. 12. Normalized relative quantities of blood transcripts with inclusion of a novel exon into HTT mRNA in infants with SMA Type 1 with weekly administration of branaplam.

FIG. 13. Median change from baseline in blood HTT mRNA levels in infants with SMA Type 1 with weekly administration of branaplam.

FIG. 14. Adaptive Ph IIb Study Design. TBD=to be determined; PBO=placebo.

FIG. 15. Simulated versus observed branaplam concentrations in plasma of Type 1 SMA patients, 33-39 months of age, after oral branaplam administration (nominal dose: 60 mg/m², 3.125 mg/kg, first-in-human proof of concept study with branaplam as described in Example 1c.1).

FIG. 16. Simulated versus observed branaplam concentrations in plasma of Type 1 SMA patients, 35-44 months of age, after oral branaplam administration (nominal dose: 60 mg/m², 3.125 mg/kg, first-in-human proof of concept study with branaplam as described in Example 1c.1).

FIG. 17. Simulated versus observed branaplam concentrations in plasma of Type 1 SMA patients, 22-29 months of age, after oral branaplam administration (nominal dose: 60 mg/m², 3.125 mg/kg, first-in-human proof of concept study with branaplam as described in Example 1c.1).

FIG. 18. Predicted distribution of mutant HTT protein in the brain cortex of BacHD mice after repeated oral branaplam administration (12 and 24 mg/kg, Example 1a). Symbols: Observed mHTT protein levels; Solid line: Median prediction; Grey area; prediction 90% confidence interval (each band corresponds to 10% confidence intervals with 9 bands).

FIG. 19. Predicted distribution of mutant HTT protein in the brain striatum of BacHD mice after repeated oral branaplam administration, 12 and 24 mg/kg, Example 1a). Symbols: Observed mHTT protein levels; Solid line: Median prediction; Grey area; prediction 90% confidence interval (each band corresponds to 10% confidence intervals with 9 bands).

FIG. 20. Timecourse of HTT protein lowering in the BacHD mouse striatum following repeat oral doses of Branaplam. P values—(Vehicle vs 24 mg/kg, 72 hrs, 3 w), **** P<0.0001, (Vehicle vs 24 mg/kg, 168 hrs, 3 w) ** P=0.0031, (Vehicle vs 24 mg/kg, 240 hrs, 3 w) ** P=0.0017.

FIG. 21. Timecourse of HTT protein lowering in the BacHD mouse cortex following repeat oral doses of Branaplam. P values—(Vehicle vs 24 mg/kg, 72 hrs) ** P=0.0060

### DETAILED DESCRIPTION OF THE INVENTION

It has been found that a splicing modulator, for example, branaplam or a pharmaceutically acceptable salt thereof, may be an ideal candidate for a treatment slowing progression of Huntington's disease, having therapeutic advantages, such as one or more of the following:
i) it is useful for the treatment of Huntington's disease as a disease-modifying therapy;
ii) it delays the onset of Huntington's disease or the onset of symptoms associated with Huntington's disease;
iii) it reduces the rate of decline of motor function associated with Huntington's disease, for example, compared to placebo, for example, as assessed by using standard scales, such as clinical scales, for example the UHDRS motor assessment scale (e.g. in Movement Disorders, 1996, 11, 136-142);
iv) it reduces the rate of cognitive decline associated with Huntington's disease, for example, compared to placebo, for example, as assessed by using standard scales, such as clinical scales [e.g. as assessed by the Symbol Digit Modalities Test, the Stroop Word Reading Test, the Montreal Cognitive Assessment or the HD Cognitive Assessment Battery (comprising the Symbol Digit Modalities Test, Trail Making Test B, One Touch Stockings, Paced Tapping, Emotion Recognition Test, Hopkins Verbal Learning Test); e.g. in Movement Disorders, 2014, 29 (10), 1281-1288];
v) it reduces the rate of psychiatric decline associated with Huntington's disease, for example, compared to placebo, for example, as assessed by using standard scales, such as clinical scales [for example the Apathy Evaluation Scale or by the Hospital Anxiety and Depression Scale; e.g. in Movement Disorders, 2016, 31 (10), 1466-1478, Movement Disorders, 2015, 30 (14), 1954-1960];
vi) it reduces the rate of decline of functional capacity associated with Huntington's disease, for example, compared to placebo, for example, as assessed by using standard scales, such as clinical scales, for example the UHDRS Total Functional Capacity, Functional Assessment and Independence scales (e.g. in Movement Disorders, 1996, 11, 136-142);
vii) it reduces the rate of progression of Huntington's disease pathophysiology [e.g. reducing the rate of brain (e.g. whole brain, caudate, striatum or cortex) volume loss (e.g. % from baseline volume) associated with Huntington's disease [e.g. as assessed by MRI, e.g. by neuroimaging measures, such as in Lancet Neurol. 2013, 12 (7), 637-649)];
viii) it reduces decline in quality of life, for example as assessed by the Huntington's Disease Health-related Quality of Life questionnaire (HDQOL) (e.g. in Movement Disorders, 2018, 33 (5), 742-749), for example compared to a sham or placebo;
ix) it has a favorable therapeutic profile, such as a favorable safety profile or metabolic profile; for example a favorable profile in relation to off-target effects, psychiatric adverse events, toxicity (e.g. genotoxicity) or cardiovascular adverse events (e.g. blood pressure, heart rate, electrocardiogrameters)

Embodiments of the present invention are described herein below:

### EMBODIMENTS (A)

Embodiment 1a: A splicing modulator for use in a treatment slowing progression of Huntington's disease.
Embodiment 2a: A splicing modulator for use in the treatment of Huntington's disease as a disease-modifying therapy.
Embodiment 3a: A splicing modulator for use in a treatment slowing the decline of motor function associated with Huntington's disease.
Embodiment 4a: A splicing modulator for use in a treatment slowing cognitive decline associated with Huntington's disease.
Embodiment 5a: A splicing modulator for use in a treatment slowing psychiatric decline associated with Huntington's disease.
Embodiment 6a: A splicing modulator for use in a treatment slowing the decline of functional capacity associated with Huntington's disease.
Embodiment 7a: A splicing modulator for use in a treatment slowing the progression of Huntington's disease pathophysiology [e.g. reducing the rate of brain (e.g. whole brain, caudate, striatum or cortex) volume loss (e.g. % from baseline volume) associated with Huntington's disease (e.g. as assessed by MRI)].
Embodiment 8a: A splicing modulator for use according to embodiment 3a, wherein motor function comprises one or more selected from the group consisting of ocular motor function, dysarthria, dystonia, chorea, postural stability and gait.
Embodiment 9a: A splicing modulator for use according to embodiment 4a, wherein cognitive decline comprises decline of one or more selected from the group consisting of attention, processing speed, visuospatial processing, timing, emotion processing, memory, verbal fluency, psychomotor function, and executive function.
Embodiment 10a: A splicing modulator for use according to embodiment 5a, wherein psychiatric decline comprises one or more selected from the group consisting of apathy, anxiety, depression, obsessive compulsive behavior, suicidal thoughts, irritability and agitation.
Embodiment 11a: A splicing modulator for use according to embodiment 6a, wherein functional capacity comprises one or more selected from the group consisting of capacity to work, capacity to handle financial affairs, capacity to manage domestic chores, capacity to perform activities of daily living, and level of care needed.
Embodiment 12a: A splicing modulator for use according to any one of embodiments 1a to 11a, wherein Huntington's disease is genetically characterized by CAG repeat expansion of from 36 to 39 (SEQ ID NO: 20) in the huntingtin gene on chromosome 4.
Embodiment 13a: A splicing modulator for use according to any one of embodiments 1a to 11a, wherein Huntington's disease is genetically characterized by CAG repeat expansion of from >39 (SEQ ID NO: 21) in the huntingtin gene on chromosome 4.
Embodiment 14a: A splicing modulator for use according to any one of embodiments 1a to 13a, wherein Huntington's disease is manifest Huntington's disease.
Embodiment 15a: A splicing modulator for use according to any one of embodiments 1a to 14a, wherein Huntington's disease is juvenile Huntington's disease or pediatric Huntington's disease.
Embodiment 16a: A splicing modulator for use according to any one of embodiments 1a to 15a, wherein Huntington's disease is early stage of Huntington's disease, middle stage of Huntington's disease, or advanced stage of Huntington's disease; in particular early stage of Huntington's disease.
Embodiment 17a: A splicing modulator for use according to any one of embodiments 1a to 16a, wherein Huntington's disease is stage I of Huntington's disease, stage II of Huntington's disease, stage III of Huntington's disease, stage IV of Huntington's disease or stage V of Huntington's disease; in particular stage I of Huntington's disease or stage II of Huntington's disease.
Embodiment 18a: A splicing modulator for use according to any one of embodiments 1a to 13a, wherein Huntington's disease is pre-manifest Huntington's disease.
Embodiment 19a: A splicing modulator for use according to any one of embodiments 1a to 18a, wherein the splicing modulator is administered according to an intermittent dosing schedule.
Embodiment 20a: A splicing modulator for use according to any one of embodiments 1a to 18a, wherein the splicing modulator is administered once a week or twice a week.
Embodiment 21a: A splicing modulator for use according to any one of embodiments 1a to 20a, wherein the splicing modulator is administered orally.
Embodiment 22a: A splicing modulator for use according to any one of embodiments 1a to 21a, wherein the splicing modulator is provided in the form of a pharmaceutical composition.
Embodiment 23a: A splicing modulator for use according to any one of embodiments 1a to 21a, wherein the splicing modulator is provided in the form of a pharmaceutical combination.
Embodiment 24a: A splicing modulator for use according to any one of embodiments 1a to 23a, wherein the splicing modulator is administered following gene therapy or treatment with an antisense compound.
Embodiment 25a: A splicing modulator for use according to any one of embodiments 1a to 24a, wherein the splicing modulator is branaplam, or a pharmaceutically acceptable salt thereof.
Embodiment 26a: A splicing modulator for use according to any one of embodiments 1a to 24a, wherein the splicing modulator is branaplam hydrochloride salt.
Embodiment 27a: A splicing modulator for use according to any one of embodiments 1a to 24a, wherein the splicing modulator is selected from the group consisting of List 4, List 5, List 6, List 7, List 8, List 9, List 10, List 11; List 12 and List 13, in particular List 4, List 5, List 6 and List 7.
Embodiment 28a: A splicing modulator for use according to any one of embodiments 1a to 24a, wherein the splicing modulator is selected from the group consisting of List 1, List 2, and List 3.
Embodiment 29a: A splicing modulator for use according to any one of embodiments 1a to 24a, wherein the splicing modulator is selected from the group consisting of List 14, List 15, List 16, List 17, List 18, List 19, List 20, List 21, List 22, List 23 and List 24.

### EMBODIMENTS (B)

Embodiment 1b: A method of treatment for slowing progression of Huntington's disease in a subject, in need thereof, comprising administering to said subject an effective amount of a splicing modulator.
Embodiment 2b: A method of treatment of Huntington's disease in a subject, in need thereof, comprising administering to said subject an effective amount of a splicing modulator as a disease-modifying therapy.
Embodiment 3b: A method of treatment for slowing the decline of motor function associated with Huntington's disease in a subject, in need thereof, comprising administering to said subject an effective amount of a splicing modulator.
Embodiment 4b: A method of treatment for slowing cognitive decline associated with Huntington's disease in a subject, in need thereof, comprising administering to said subject an effective amount of a splicing modulator.
Embodiment 5b: A method of treatment for slowing psychiatric decline associated with Huntington's disease in a subject, in need thereof, comprising administering to said subject an effective amount of a splicing modulator.
Embodiment 6b: A method of treatment for slowing the decline of functional capacity associated with Huntington's disease in a subject, in need thereof, comprising administering to said subject an effective amount of a splicing modulator.
Embodiment 7b: A method of treatment for slowing the progression of Huntington's disease pathophysiology [e.g. reducing the rate of brain (e.g. whole brain, caudate, striatum or cortex) volume loss (e.g. % from baseline volume) associated with Huntington's disease (e.g. as assessed by MRI)] in a subject, in need thereof, comprising administering to said subject an effective amount of a splicing modulator.
Embodiment 8b: The method according to embodiment 3b, wherein motor function comprises one or more selected from the group consisting of ocular motor function, dysarthria, dystonia, chorea, postural stability and gait.
Embodiment 9b: The method according to embodiment 4b, wherein cognitive decline comprises decline of one or more selected from the group consisting of attention, processing speed, visuospatial processing, timing, emotion processing, memory, verbal fluency, psychomotor function, and executive function.
Embodiment 10b: The method according to embodiment 5b, wherein psychiatric decline comprises one or more selected from the group consisting of apathy, anxiety, depression, obsessive compulsive behavior, suicidal thoughts, irritability and agitation.
Embodiment 11b: The method according to embodiment 6b, wherein functional capacity comprises one or more selected from the group consisting of capacity to work, capacity to handle financial affairs, capacity to manage domestic chores, capacity to perform activities of daily living, and level of care needed.
Embodiment 12b: The method according to any one of embodiments 1b to 11b, wherein Huntington's disease is genetically characterized by CAG repeat expansion of from 36 to 39 (SEQ ID NO: 20) in the huntingtin gene on chromosome 4.
Embodiment 13b: The method according to any one of embodiments 1b to 11b, wherein Huntington's disease is genetically characterized by CAG repeat expansion of from >39 (SEQ ID NO: 21) in the huntingtin gene on chromosome 4.
Embodiment 14b: The method according to any one of embodiments 1b to 13b, wherein Huntington's disease is manifest Huntington's disease.
Embodiment 15b: The method according to any one of embodiments 1b to 14b, wherein Huntington's disease is juvenile Huntington's disease or pediatric Huntington's disease.
Embodiment 16b: The method according to any one of embodiments 1b to 15b, wherein Huntington's disease is early stage of Huntington's disease, middle stage of Huntington's disease, or advanced stage of Huntington's disease; in particular early stage of Huntington's disease.
Embodiment 17b: The method according to any one of embodiments 1b to 16b, wherein Huntington's disease is stage I of Huntington's disease, stage II of Huntington's disease, stage III of Huntington's disease, stage IV of Huntington's disease or stage V of Huntington's disease; in particular stage I of Huntington's disease or stage II of Huntington's disease.
Embodiment 18b: The method according to any one of embodiments 1b to 13b, wherein Huntington's disease is pre-manifest Huntington's disease.
Embodiment 19b: The method according to any one of embodiments 1b to 18b, wherein the splicing modulator is administered according to an intermittent dosing schedule.
Embodiment 20b: The method according to any one of embodiments 1b to 18b, wherein the splicing modulator is administered once a week or twice a week.
Embodiment 21b: The method according to any one of embodiments 1b to 20b, wherein the splicing modulator is administered orally.
Embodiment 22b: The method according to any one of embodiments 1b to 21b, wherein the splicing modulator is provided in the form of a pharmaceutical composition.
Embodiment 23b: The method according to any one of embodiments 1b to 21b, wherein the splicing modulator is provided in the form of a pharmaceutical combination.
Embodiment 24b: The method according to any one of embodiments 1b to 23b, wherein the splicing modulator is administered following gene therapy or treatment with an antisense compound.
Embodiment 25b: The method according to any one of embodiments 1b to 24b, wherein the splicing modulator is branaplam, or a pharmaceutically acceptable salt thereof.
Embodiment 26b: The method according to any one of embodiments 1b to 24b, wherein the splicing modulator is branaplam hydrochloride salt.
Embodiment 27b: The method according to any one of embodiments 1b to 24b, wherein the splicing modulator is selected from the group consisting of List 4, List 5, List 6, List 7, List 8, List 9, List 10, List 11, List 12 and List 13; in particular List 4, List 5, List 6 and List 7.
Embodiment 28b: The method according to any one of embodiments 1b to 24b, wherein the splicing modulator is selected from the group consisting of List 1, List 2 and List 3.
Embodiment 29b: The method according to any one of embodiments 1b to 24b, wherein the splicing modulator is selected from the group consisting of List 14, List 15, List 16, List 17, List 18, List 19, List 20, List 21, List 22, List 23 and List 24.

### EMBODIMENTS (C)

It has been surprisingly found that branaplam promotes the inclusion of a novel, 115-bp exon containing an in-frame stop codon (55 bp from the 3′ end of the novel exon) between exons 49 and 50 of the HTT mRNA thereby lowering HTT transcript and protein levels. Thus, in another aspect, the invention relates to:
Embodiment 1c: A method of treatment for slowing progression of Huntington's disease in a subject, in need thereof, comprising administering to said subject an effective amount of a splicing modulator, such as branaplam or a pharmaceutically acceptable salt thereof, by producing an in-frame stop codon between exons 49 and 50 in the HTT mRNA. For example, the splicing modulator is selected from the group consisting of List1, List 2, List 3, List 4, List 5, List 6, List 7, List 8, List 9, List 10, List 11, List 12, List 13, List 14, List 15, List 16, List 17, List 18, List 19, List 20, List 21, List 22, List 23 and List 24.
Embodiment 2c: A splicing modulator, such as branaplam or a pharmaceutically acceptable salt thereof, for use in a treatment slowing progression of Huntington's disease by producing an in-frame stop codon between exons 49 and 50 in the HTT mRNA. For example, the splicing modulator is selected from the group consisting of List1, List 2, List 3, List 4, List 5, List 6, List 7, List 8, List 9, List 10, List 11, List 12, List 13, List 14, List 15, List 16, List 17, List 18, List 19, List 20, List 21, List 22, List 23 and List 24.
Embodiment 3c: The method according to embodiment 1c, wherein the splicing modulator is branaplam, or a pharmaceutically acceptable salt thereof, such as branaplam hydrochloride salt.
Embodiment 4c: A splicing modulator for use according to embodiment 2c, wherein the splicing modulator is branaplam, or a pharmaceutically acceptable salt thereof, such as branaplam hydrochloride salt.

### General Definition of Terms

The term “HD” or “Huntington's disease”, as used herein, refers to the neurodegenerative disorder, characterized by motor, cognitive, psychiatric and functional capacity decline, and caused by CAG repeat expansions in the huntingtin gene.

The term “manifest HD” or “manifest Huntington's disease”, as used herein, refers to having diagnosis of HD as clinically established {e.g. on the basis of: confirmed family history or positive genetic test (confirmation of CAG repeat expansion≥36 (SEQ ID NO: 22)); and onset of motor disturbances [diagnostic confidence score (DCS) of 4, as defined by the Unified Huntington Rating Scale (UHDRS) total motor score (TMS)]}. In one embodiment, the term “manifest HD” or “manifest Huntington's disease”, as used herein, refers to a patient having diagnosis of HD as clinically established {e.g. on the basis of: confirmed family history or positive genetic test (confirmation of CAG repeat expansion≥36 (SEQ ID NO: 22)); and onset of motor disturbances [diagnostic confidence score (DCS) of 4, as defined by the Unified Huntington Rating Scale (UHDRS) total motor score (TMS)]}.

The term “pre-manifest HD” or “pre-manifest Huntington's disease”, as used herein, refers to having genetic diagnosis of HD {e.g. on the basis of: positive genetic test (confirmation of CAG repeat expansion≥40 (SEQ ID NO: 21))} without onset of motor disturbances as clinically stablished, for example, as assessed according to standard scales, such as, clinical scales [e.g. on the basis of a diagnostic confidence score (DCS) of <4, as defined by the Unified Huntington Rating Scale (UHDRS) total motor score (TMS)]. In one embodiment, term “pre-manifest HD” or “pre-manifest Huntington's disease”, as used herein, refers to a patient having genetic diagnosis of HD {e.g. on the basis of: positive genetic test (confirmation of CAG repeat expansion≥40 (SEQ ID NO: 21))} without onset of motor disturbances as clinically stablished, for example, as assessed according to standard scales, such as, clinical scales [e.g. on the basis of a diagnostic confidence score (DCS) of <4, as defined by the Unified Huntington Rating Scale (UHDRS) total motor score (TMS)].

In one embodiment, the term “slowing progression of HD”, “slowing progression of Huntington's disease”, “to slow the progression of HD” or “to slow the progression of Huntington's disease”, as used herein, refers to, for example:
reducing the rate of Huntington's disease progression (e.g. reducing the rate of progression between stages of Huntington's disease);
delaying the onset of Huntington's disease;
delaying the onset of symptoms associated with Huntington's disease;
reducing the rate of progression (e.g. reducing the annual rate of decline) of symptoms (e.g. one or more symptoms) associated with Huntington's disease; or
reducing the rate of progression of Huntington's disease pathophysiology;
   (e.g. compared to placebo or compared to natural history control group; e.g. according to standard scales, such as clinical scales, herein above or below, or according to neuroimaging measures).

In one embodiment, the term “rate of progression”, as used herein, refers, for example, to the annual rate of change (e.g. decline) or the rate of change (e.g. decline) per year, for example as assessed according to standard scales, such as clinical scales, or according to neuroimaging measures.

The term “reducing”, as used herein, refers to e.g. 5%, 10%, 20%, 30%, 40%, 50%, 60% or 70% reduction, for example, per year of treatment.

The term “delaying”, as used herein, refers to delay for at least e.g. 0.5, 1, 2, 3, 4, 5, 6, 7, 8, 9, 10, 11, 12, 13, 14 or 15 years.

In one embodiment, the term “slowing progression of HD”, “slowing progression of Huntington's disease”, “to slow the progression of HD” or “to slow the progression of Huntington's disease”, as used herein, refers to delaying the onset of Huntington's disease, e.g. increasing time for the onset of Huntington's disease as defined herein. In another embodiment, it refers to reducing the rate of progression between stages of Huntington's disease, for example, reducing the rate of progression from an initial stage of HD into a more advanced stage of HD, as assessed, for example, compared to placebo, according to standard scales, such as clinical scales [e.g. according to the UHDRS total functional capacity (TFC) scale, for example, in Neurology, 1979, 29, 1-3]. In one embodiment, it refers to reducing the rate of progression from stage 1 of HD into stage 2 of HD (e.g. compared to placebo). In another embodiment, it refers to reducing the rate of progression from stage 2 of HD into stage 3 of HD (e.g. compared to placebo). In another embodiment, it refers to reducing the rate of progression from stage 3 of HD into stage 4 of HD (e.g. compared to placebo). In another embodiment, it refers to reducing the rate of progression from stage 4 of HD into stage 5 of HD (e.g. compared to placebo). In a further embodiment, it refers to reducing the rate of progression from early HD into middle stage HD (e.g. compared to placebo). In a further embodiment, it refers to reducing the rate of progression from middle stage HD into advanced HD (e.g. compared to placebo). The term “reducing the rate of progression”, as used herein, refers, for example, to increasing time for progression of stage of HD (e.g. compared to placebo).

In another embodiment, the term “slowing progression of HD”, “slowing progression of Huntington's disease”, “to slow the progression of HD” or “to slow the progression of Huntington's disease”, as used herein, refers to delaying the onset of Huntington's disease (e.g. increasing time for the onset of Huntington's disease as defined herein) by at least 25% (e.g. by 25% or more, such as from 25% to 50%).

The term “onset of Huntington's disease”, as used herein, refers to clinical diagnosis of HD as generally established [e.g. onset of motor disturbances based on diagnostic confidence score (DCS) of 4, as defined by the Unified Huntington Rating Scale (UHDRS) total motor score (TMS)].

In another embodiment, the term “slowing progression of HD”, “slowing progression of Huntington's disease”, “to slow the progression of HD” or “to slow the progression of Huntington's disease”, as used herein, refers to delaying the onset of symptoms associated with Huntington's disease, e.g. increasing time for the onset of one or more symptom associated with Huntington's disease selected from decline of motor function associated with Huntington's disease, cognitive decline associated with Huntington's disease, psychiatric decline associated with Huntington's disease and decline of functional capacity associated with Huntington's disease, as defined herein. In another embodiment, it refers to reducing the rate of progression of one or more symptom associated with Huntington's disease selected from decline of motor function associated with Huntington's disease, cognitive decline associated with Huntington's disease, psychiatric decline associated with Huntington's disease and decline of functional capacity associated with Huntington's disease, as defined herein. The term “reducing the rate of”, as used herein, refers, for example, to increasing time for onset or increasing time for a rise of severity (e.g. compared to placebo). In one embodiment, the term “slowing progression of HD”, “slowing progression of Huntington's disease”, “to slow the progression of HD” or “to slow the progression of Huntington's disease”, as used herein, refers to reducing the rate of progression of pre-manifest HD into manifest HD [i.e. delaying the onset of manifest HD; e.g. compared to placebo; e.g. as assessed by a diagnostic confidence score (DCS) of 4, as defined by the Unified Huntington Rating Scale (UHDRS) total motor score (TMS)].

In a further embodiment, the term “slowing progression of HD”, “slowing progression of Huntington's disease”, “to slow the progression of HD” or “to slow the progression of Huntington's disease”, as used herein, refers to slowing the progression of Huntington's disease pathophysiology.

The term “slowing the progression of Huntington's disease pathophysiology”, as used herein, refers to reducing the rate of progression of Huntington's disease pathophysiology, for example, as assessed by magnetic resonance imaging (MRI) [e.g. by neuroimaging measures, such as in Lancet Neurol. 2013, 12 (7), 637-649]. For example, it refers to reducing the rate (e.g. reducing the annual rate, for example, versus placebo) of brain (e.g. whole brain, caudate, striatum or cortex) volume loss (e.g. % from baseline volume) associated with Huntington's disease (e.g. as assessed by MRI).

The term “motor function”, as used herein, refers to motor features of HD comprising, for example, one or more selected from the group consisting of ocular motor function, dysarthria, chorea, postural stability and gait.

The term “decline of motor function”, as used herein, refers to decreased motor function (e.g. from normal motor function or from previous clinic visit). Decline of motor function may be assessed, for example, according to standard scales, such as clinical scales (e.g. UHDRS motor assessment scale, as measured by the UHDRS Total Motors Score; e.g. in Movement Disorders, 1996, 11, 136-142).

The term “slowing the decline of motor function” or “to slow the decline of motor function”, as used herein, refers to reducing the rate of decline of motor function (e.g. compared to placebo; e.g. reduction in the annual rate of decline of motor function, for example, versus placebo; e.g. as assessed by the UHDRS Total Motors Score). The term “reducing the rate”, as used herein, refers to increasing time for onset or increasing time for a rise of severity (e.g. compared to placebo; e.g. reduction in the annual rate of decline, for example, versus placebo).

The term “cognitive decline”, as used herein, refers to decreased cognitive abilities (e.g. from normal cognition function or from previous clinic visit). In one embodiment, it comprises, for example, decline of one or more selected from the group consisting of attention, processing speed, visuospatial processing, timing, emotion processing, memory, verbal fluency, psychomotor function, and executive function. Cognitive decline may be assessed, for example, according to standard scales, such as clinical scales [e.g. as assessed by the Symbol Digit Modalities Test, the Stroop Word Reading Test, the Montreal Cognitive Assessment or the HD Cognitive Assessment Battery (comprising the Symbol Digit Modalities Test, Trail Making Test B, One Touch Stockings, Paced Tapping, Emotion Recognition Test, Hopkins Verbal Learning Test); e.g. in Movement Disorders, 2014, 29 (10), 1281-1288].

The term “slowing cognitive decline” or “to slow cognitive decline”, as used herein, refers to reducing the rate of cognitive decline (e.g. compared to placebo; e.g. reduction in the annual rate of cognitive decline versus placebo; e.g. as assessed by the Symbol Digit Modalities Test, by the Stroop Word Reading Test, by the Montreal Cognitive Assessment or by the HD Cognitive Assessment Battery). The term “reducing the rate”, as used herein, refers to increasing time for onset or increasing time for a rise of severity (e.g. compared to placebo; e.g. reduction in the annual rate of decline, for example, versus placebo).

The term “psychiatric decline”, as used herein, refers to decreased psychiatric function (e.g. from normal psychiatric function or from previous clinic visit). In one embodiment, it comprises, for example, one or more selected from the group consisting of apathy, anxiety, depression obsessive compulsive behavior, suicidal thoughts, irritability and agitation. Psychiatric decline may be assessed, for example, according to standard scales, such as clinical scales (e.g. as assessed by the Apathy Evaluation Scale or by the Hospital Anxiety and Depression Scale; e.g. in Movement Disorders, 2016, 31 (10), 1466-1478, Movement Disorders, 2015, 30 (14), 1954-1960).

The term “slowing psychiatric decline” or “to slow psychiatric decline”, as used herein, refers to reducing the rate of psychiatric decline (e.g. compared to placebo; e.g. reduction in the annual rate of psychiatric decline versus placebo; e.g. as assessed by the Apathy Evaluation Scale or by the Hospital Anxiety and Depression Scale). The term “reducing the rate”, as used herein, refers to increasing time for onset or increasing time for a rise of severity (e.g. compared to placebo; e.g. reduction in the annual rate of decline, for example, versus placebo).

The term “functional capacity”, as used herein, refers, for example, to the ability to work, handle financial affairs, manage domestic chores, perform activities of daily living, and level of care needed. Functional capacity comprises, for example, one or more selected from the group consisting of capacity to work, capacity to handle financial affairs, capacity to manage domestic chores, capacity to perform activities of daily living, and level of care needed.

The term “decline of functional capacity”, as used herein, refers to decreased functional capacity (e.g. from normal functional capacity or from previous clinic visit). Decline of functional capacity may be assessed, for example, according to standard scales, such as clinical scales (e.g. UHDRS functional assessment scale and independence scale, and UHDRS Total Functional Capacity Scale e.g. in Movement Disorders, 1996, 11, 136-142).

The term “slowing the decline of functional capacity” or “to slow the decline of functional capacity”, as used herein, refers to reducing the rate of decline of functional capacity (e.g. compared to placebo; e.g. reduction in the annual rate of decline of functional capacity versus placebo; e.g. as assessed by the UHDRS functional assessment scale and independence scale or by the UHDRS Total Functional Capacity Scale). The term “reducing the rate”, as used herein, refers to increasing time for onset or increasing time for a rise of severity (e.g. compared to placebo; e.g. reduction in the annual rate of decline, for example, versus placebo).

The term “decline”, as used herein, refers, for example, to worsening over time (e.g. annually or per year) of a condition or of a particular feature of a condition, for example as assessed according to standard scales, such as clinical scales.

The term “Unified Huntington Disease Rating Scale” or “UHDRS” as used herein, refers to the clinical rating scale developed by the Huntington Study Group (e.g. in Movement Disorders, 1996, 11, 136-142, which is incorporated fully herein by reference), which assesses domains of clinical performance and capacity in HD. It comprises rating scales for motor function, cognitive function and functional capacity. It yields scores assessing primary features of HD (e.g. motor and cognitive) and overall functional impact of these features. The term “cHDRS” refers to the composite Unified Huntington Disease Rating Scale, which provides composite measure of motor, cognitive and global functioning (e.g. in Neurology, 2017, 89, 2495-2502).

The term “HD stage 1”, “HD stage I”, “Huntington's disease stage 1”, “Huntington's disease stage I”, “stage 1 of Huntington's disease” or “stage I of Huntington's disease”, as used herein, refers to a disease stage of HD as clinically stablished [e.g. as assessed according to standard scales, for example, clinical scales, such as on the basis of the UHDRS total functional capacity (TFC) scale, wherein the TFC score is of 11 to 13]. At HD stage 1, typically, the patient has been clinically diagnosed with HD, is fully functional at home and at work and maintains independence as regards functional capacities; typically 0 to 8 years from onset of Huntington's disease.

The term “HD stage 2”, “HD stage II”, “Huntington's disease stage 2”, “Huntington's disease stage II”, “stage 2 of Huntington's disease” or “stage II of Huntington's disease”, as used herein, refers to a disease stage of HD as clinically stablished [e.g. as assessed according to standard scales, for example, clinical scales, such as on the basis of the UHDRS total functional capacity (TFC) scale, wherein the TFC score is of 7 to 10]. At HD stage 2, typically, the patient is still functional at work, however at lower capacity, is mostly able to carry out daily activities, despite some difficulties, and usually requires only slight assistance; typically 3 to 13 years from onset of Huntington's disease.

The term “HD stage 3”, “HD stage III”, “Huntington's disease stage 3”, “Huntington's disease stage III”, “stage 3 of Huntington's disease” or “stage III of Huntington's disease”, as used herein, refers to a disease stage of HD as clinically stablished [e.g. as assessed according to standard scales, for example, clinical scales, such as on the basis of the UHDRS total functional capacity (TFC) scale, wherein the TFC score is of 4 to 6]. At HD stage 3, typically, the patient can no longer conduct work or manage household chores, requires substantial help for daily financial affairs, domestic responsibilities, and activities of daily living; typically 5 to 16 years from onset of Huntington's disease.

The term “HD stage 4”, “HD stage IV”, “Huntington's disease stage 4”, “Huntington's disease stage IV”, “stage 4 of Huntington's disease” or “stage IV of Huntington's disease”, as used herein, refers to a disease stage of HD as clinically stablished [e.g. as assessed according to standard scales, for example, clinical scales, such as on the basis of the UHDRS total functional capacity (TFC) scale, wherein the TFC score is of 1 to 3]. At HD stage 4, typically, the patient is not independent, but still can reside at home with help from either family or professionals, however, requiring substantial assistance in financial affairs, domestic chores, and most activities of daily living; typically 9 to 21 years from onset of Huntington's disease.

The term “HD stage 5”, “HD stage V”, “Huntington's disease stage 5”, “Huntington's disease stage V”, “stage 5 of Huntington's disease” or “stage V of Huntington's disease”, as used herein, refers to a disease stage of HD as clinically stablished [e.g. as assessed according to standard scales, for example, clinical scales, such as on the basis of the UHDRS total functional capacity (TFC) scale, wherein the TFC score is of 0]. At HD stage 5, typically, the patient needs total support in daily activities from professional nursing care; typically 11 to 26 years from onset of Huntington's disease.

The term “early HD”, “early Huntington's disease”, “early stage of HD” or “early stage of Huntington's disease”, as used herein, refers to a disease stage of HD, wherein the patient is largely functional and may continue to work and live independently, despite suffering from, for example, one or more selected from the group consisting of minor involuntary movements, subtle loss of coordination and difficulty thinking through complex problems. In one embodiment, early HD″, “early Huntington's disease”, “early stage of HD” or “early stage of Huntington's disease” refers to “HD stage 1” or “HD stage 2”, as defined herein.

The term “moderate HD”, “moderate Huntington's disease”, “moderate stage of HD”, “moderate stage of Huntington's disease”, “middle stage HD”, “middle stage Huntington's disease”, “middle stage of HD” or “middle stage of Huntington's disease”, as used herein, refers to a disease stage of HD, wherein the patient may no be able to work, manage own finances or perform own household chores, but will be able to eat, dress, and attend to personal hygiene with assistance. Typically, at this stage, for example, chorea may be prominent, as well as problems with swallowing, balance, falls, weight loss, and problem solving. In one embodiment, moderate HD″, “moderate Huntington's disease”, “moderate stage of HD”, “moderate stage of Huntington's disease”, “middle stage HD”, “middle stage Huntington's disease”, “middle stage of HD” or “middle stage of Huntington's disease” refers to “HD stage 3”, as defined herein.

The term “advanced HD”, “advanced Huntington's disease”, “advanced stage of HD”, “advanced stage of Huntington's disease”, “late HD” or “late Huntington's disease”, “late stage of HD” or “late stage of Huntington's disease”, as used herein, refers to a disease stage of HD, wherein the patient requires assistance in all activities of daily living. Typically, at this stage, for example, chorea may be severe, but more often it is replaced by rigidity, dystonia, and bradykinesia. In one embodiment, “advanced HD”, “advanced Huntington's disease”, “advanced stage of HD”, “advanced stage of Huntington's disease”, “late HD” or “late Huntington's disease”, “late stage of HD” or “late stage of Huntington's disease” refers to “HD stage 4” or “HD stage 5”, as defined herein.

The term “juvenile HD” or “juvenile Huntington's disease”, as used herein, refers to diagnosis of HD as clinically stablished {e.g. on the basis of: confirmed family history or positive genetic test (i.e. confirmation of CAG repeat expansion≥36 (SEQ ID NO: 22)); and onset of symptoms by age <21 years}. In one embodiment, the term “juvenile HD” or “juvenile Huntington's disease”, as used herein, refers to a patient affected by HD {e.g. on the basis of: confirmed family history or positive genetic test (i.e. confirmation of CAG repeat expansion≥36 (SEQ ID NO: 22))} and who has onset of symptoms by age <21 years.

The term “pediatric HD” or “pediatric Huntington's disease”, as used herein, refers to a patient affected by HD {e.g. on the basis of: confirmed family history or positive genetic test (i.e. confirmation of CAG repeat expansion≥36 (SEQ ID NO: 22)) and clinical diagnosis} and who is aged <18 years.

The term “HD patient”, “Huntington's disease patient”, “patient with Huntington's disease” or “patient with HD” refers to a patient with HD, as defined herein.

The term “treat” “treating” “treatment” or “therapy”, as used herein, means obtaining beneficial or desired results, for example, clinical results. Beneficial or desired results can include, but are not limited to, stabilizing or improving progression of stage of HD (e.g. compared to placebo). One aspect of the treatment is, for example, that said treatment should have a minimal adverse effect on the patient, e.g. the agent used should have a high level of safety, for example without producing adverse side effects. In one embodiment, the term “method for the treatment”, as used herein, refers to “method to treat”.

The term “intermittent dosing regimen” or “intermittent dosing schedule”, as used herein, means a dosing regimen that comprises administering a splicing modulator, such as those defined herein, followed by a resting period. For example, the splicing modulator is administered according to an intermittent dosing schedule of at least two cycles, each cycle comprising (a) a dosing period and thereafter (b) a resting period. As used herein, the term “resting period” refers, in particular, to a period of time during which the patient is not given the splicing modulator (i.e., a period of time wherein the treatment with the splicing modulator is withheld). For example, if a splicing modulator, such as those defined herein, is given on a daily basis, there would be rest period if the daily administration is discontinued for some time, e.g., for some number of days, or the plasma concentration of the splicing modulator is maintained at sub-therapeutic level for some time e.g., for some number of days. The dosing period and/or the dose of the splicing modulator can be the same or different between cycles. The total treatment time (i.e., the number of cycles for treatment) may also vary from patient to patient based, for example, on the particular patient being treated (e.g., Stage I HD patient). In one embodiment, an intermittent dosing schedule comprises at least two cycles, each cycle comprising (a) a dosing period during which a therapeutically effective amount of the splicing modulator is administered to said patient and thereafter (b) a resting period. The term “intermittent dosing regimen” or “intermittent dosing schedule”, as used herein, refers to both a dosing regimen for administering the splicing modulator alone (i.e. monotherapy) or a dosing regimen for administering the splicing modulator in combination with at least a further active ingredient (i.e. combination therapy). In one embodiment, the term “intermittent dosing regimen” or “intermittent dosing schedule” refers to repeated on/off treatment, wherein the splicing modulator is administered at regular intervals in a periodic manner, for example, once a week, every 3 days, every 4 days or twice a week.

The term “once a week” or “once weekly” or “QW” in the context of administering a drug means herein administering one dose of a drug once each week, wherein the dose is, for example, administered on the same day of the week. In one embodiment, the term administering or administration of branaplam once a week, as used herein above or below, in particular in the embodiments and the claims, refers to branaplam administered in an amount, for example, of from 50 mg to 200 mg once a week, such as 140 mg once a week, of from 200 mg to 400 mg once a week, such as 280 mg once a week, or of from 400 mg to 700 mg once a week, such as 560 mg once a week.

The term “twice a week” or “twice weekly” or “BIW” in the context of administering a drug means herein administering one dose of a drug twice each week, wherein each administration is, for example, on separate days, for example, at regular intervals of, for example, 72 hours. In one embodiment, the term administering or administration of branaplam twice a week, as used herein above or below, in particular in the embodiments and the claims, refers branaplam administered in an amount, for example, of from 25 mg to 100 mg twice a week, such as 70 mg twice a week, of from 100 mg to 200 mg twice a week, such as 140 mg twice a week, or of from 200 mg to 350 mg twice a week, such as 280 mg twice a week.

As used herein, reference to an amount (e.g. mg, mg/ml, mg/m², percentage) of branaplam, or a pharmaceutical acceptable salt thereof, is to be understood to refer the amount of the compound of formula (I), as herein below, in the free form, which will be adapted accordingly for a pharmaceutically acceptable salt thereof, for example hydrochloride salt thereof (e.g. branaplam hydrochloride monohydrate).

As used herein, the terms “free form” or “free forms” or “in free form” or “in the free form” refers to the compound in non-salt form, such as the base free form.

The term “about” in relation to a numerical value X means, for example, X±15%, including all the values within this range.

The term “disease-modifying therapy” or disease-modifying treatment”, as used herein, refers to a drug that can modify or change the course of a condition or a disorder or a disease (i.e. a disease-modifying drug), such as HD, as defined herein.

As used herein, the term “subject” refers to a mammalian organism, preferably a human being (male or female).

As used herein, the term “patient” refers to a subject who is diseased and would benefit from the treatment.

As used herein, a subject is “in need of” a treatment if such subject (patient) would benefit biologically, medically or in quality of life from such treatment.

The term “a therapeutically effective amount” or “an effective amount” of a compound of the present invention refers to an amount of a compound of the present invention that will elicit the biological or medical response of a subject. In another embodiment, the term refers to the amount of the compound of the present invention that, when administered to a subject, is effective to at least partially ameliorate a condition, or a disorder or a disease.

The term “one or more” refers to either one or a number above one (e.g. 2, 3, 4, 5, etc.).

The term “List 1”, as used herein above and below [e.g. in Embodiments (A) or (B)], refers to compounds disclosed in WO2014/028459, which are incorporated herein by reference, such as compounds of the Examples and claims (e.g. compounds according to any one of claims 1 to 14, each of which are herein incorporated by reference, for example, taken as such or in a combination thereof), or pharmaceutically acceptable salts thereof. In one embodiment, it refers to compounds of the Examples of WO2014/028459, or pharmaceutically acceptable salts thereof, such as Examples 1-1, 1-2, 1-3, 1-4, 1-5, 1-6, 1-7, 1-8, 1-9, 1-10, 1-11, 1-12, 1-13, 1-4, 1-5, 1-16, 1-17, 1-8, 1-19, 1-20, 1-21, 1-22, 2-1, 2-2, 2-3, 3-1, 3-2, 3-3, 3-4, 3-5, 3-6, 3-7, 3-8, 3-9, 3-10, 3-11, 3-12, 4-1, 5-1, 6-1, 7-1, 8-1, 9-1, 10-1, 11-1, 12-1, 13-1, 14-1, 15-1, 16-1, 16-1, 16-1, 16-4, 16-5, 17-1, 17-2, 17-3, 17-4, 17-5, 17-6, 17-7, 17-8, 17-9, 17-10, 17-11, 17-12, 17-13, 18-1, 18-2, 18-3, 18-4, 18-5, 18-6, 18-7, 18-8, 18-9, 18-10, 18-11, 18-12, 18-13, 18-14, 18-15, 18-16, 18-17, 18-18, 19-1, 19-2, 19-3, 19-4, 19-5, 19-6, 19-7, 20-1, 20-2, 20-3, 20-4, 20-5, 20-6, 20-7, 20-8, 20-9, 21-1, 21-2, 22-1, 23-1, 24-1, 24-2, 24-3, 24-4, -5, 24-6, 24-7, 24-8, 24-1, 24-2, 24-3, 24-4, 24-5, 24-6, 24-7, 24-8, 24-9, 24-10, 24-11, 24-12, 25-1, 25-2, 25-3, 25-4, 25-5, 25-6, 26-1, 26-2, 26-3, 26-4, 26-5, 27-2, 27-2, 27-3, 28-1, 29-1, 30-1, 30-2, 31-1, 32-1, 32-2, 32-3, 32-4, 32-5, 32-6, 32-7, 32-8, 32-9, 33-1, 34-1, 34-1, 34-2, 34-3, 34-4, 34-5, 35-1, 35-1, 35-2, 35-3, 35-4, 35-5, 35-6, 36-1, 37-1, 38-1, 39-1, 39-2, 40-1, 40-2, 40-3, 40-4, 40-5, 40-6, 40-7, 40-8, 41-1, 41-2, 41-3, 41-4, 41-5, 41-6, 41-7, 41-8, 41-9, 41-10, 41-11, 41-12, 41-13, 41-14, 41-15, 41-16, 41-17, 41-18, 41-19, 41-20, 42-1, 42-2, 42-3, 42-4, 42-5, 42-6, 42-7, 42-8, 42-9, 42-10, 42-11, 43-1, 43-2, 43-3, 43-4, 43-5, 43-6 or 43-7, each of which are herein incorporated by reference, or pharmaceutically acceptable salts thereof. In another embodiment, it refers to one or more compounds selected from claim 14 of WO2014/028459, each of which are herein incorporated by reference, or pharmaceutically acceptable salts thereof.

The term “List 2”, as used herein above and below [e.g. in Embodiments (A) or (B)], refers to compounds disclosed in WO2014/116845, which are incorporated herein by reference, such as compounds of the Examples and claims (e.g. compounds according to any one of claims 1 to 12, each of which are herein incorporated by reference, for example, taken as such or in a combination thereof), or pharmaceutically acceptable salts thereof. In one embodiment, it refers to compounds of Examples 1 to 12 or 15 to 109 of WO2014/116845, each of which are herein incorporated by reference, or pharmaceutically acceptable salts thereof. In another embodiment, it refers to one or more compounds selected from claim 12 of WO2014/116845, each of which are herein incorporated by reference, or pharmaceutically acceptable salts thereof.

The term “List 3”, as used herein above and below [e.g. in Embodiments (A) or (B)], refers to compounds disclosed in WO2015/017589, which are incorporated herein by reference, such as compounds of the Examples and claims (e.g. compounds according to any one of claims 1 to 12, each of which are herein incorporated by reference, for example, taken as such or in a combination thereof), or pharmaceutically acceptable salts thereof. In one embodiment, it refers to compounds of the Examples of WO2015/017589, or pharmaceutically acceptable salts thereof, such as Examples 1-1, 1-2, 1-3, 2-1, 3-1, 3-2, 3-3, 3-4, 3-5, 3-6, 3-7, 4-1, 5-1, 6-1, 6-2, 7-1, 7-2, 7-3, 7-4, 7-5, 7-6, 8-1, 8-2, 9-1, 9-2, 10-1, 10-2, 10-3, 10-4, 10-5, 11-1, 12-1, 13-1, 14-1, 15-1, 15-2, 15-3, 16-1, 17-1, 18-1, 18-2, 18-3, 19-1, 20-1, 20-2, 20-3, 20-4, 20-5, 20-6, 22-1, 23-1, 24-1, 25-1, 26-1, 26-2, 26-3, 26-4, 26-5, 26-6, 26-7, 27-1, 27-2, 27-3, 28-1, 29-1, 29-2, 29-3, 30-1, 31-1, 32-1, 33-1, 34-1, 34-2, 35-1, 35-2, 35-3, 35-4, 36-1, 36-2, 36-3, 36-4, 36-5, 36-6 or 37-1, each of which are herein incorporated by reference, or pharmaceutically acceptable salts thereof. In another embodiment, it refers to one or more compounds selected from claim 12 of WO2015/017589, each of which are herein incorporated by reference, or pharmaceutically acceptable salts thereof.

The term “List 4”, as used herein above and below [e.g. in Embodiments (A) or (B)], refers to compounds disclosed in WO2017/100726, which are incorporated herein by reference, such as compounds of the Examples and claims (e.g. compounds according to any one of claims 1 to 9, each of which are herein incorporated by reference), or pharmaceutically acceptable salts thereof. In one embodiment, it refers to any one of compounds 1 to 465 of WO2017/100726, each of which are herein incorporated by reference, or pharmaceutically acceptable salts thereof, In one embodiment, it refers to compounds 26, 31, 47, 258, 307, 352, 424, 425, 426, 427, 428, 429, 430, 431, 432 or 433, of, WO2017/100726, each of which are herein incorporated by reference, or pharmaceutically acceptable salts thereof. In one embodiment, it refers to compounds 44, 46, 48, 51, 63, 64, 170, 176, 200, 212, 218, 315, 318, 348, 350, 393, 411, 412, 413, 414, 415, 416, 417, 418, 419, 420, 421, 422 or 423, of WO2017/100726, each of which are herein incorporated by reference, or pharmaceutically acceptable salts thereof. In another embodiment, it refers to one or more compounds selected from claim 6 of WO2017/100726, each of which are herein incorporated by reference, or pharmaceutically acceptable salts thereof. In another embodiment, it refers to one or more compounds selected from claim 7 of WO2017/100726, each of which are herein incorporated by reference, or pharmaceutically acceptable salts thereof. In another embodiment, it refers to one or more compounds selected from claim 8 of WO2017/100726, each of which are herein incorporated by reference, or pharmaceutically acceptable salts thereof. In another embodiment, it refers to one or more compounds selected from claim 9 of WO2017/100726, each of which are herein incorporated by reference, or pharmaceutically acceptable salts thereof.

The term “List 5”, as used herein above and below [e.g. in Embodiments (A) or (B)], refers to compounds disclosed in WO2018/226622, which are incorporated herein by reference, such as compounds of the Examples and claims (e.g. compounds according to any one of claims 1 to 7, each of which are herein incorporated by reference), or pharmaceutically acceptable salts thereof. In one embodiment, it refers to compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 714, 175, 176, 177, 178, 179, 180, 181, 182, 183, 184, 185, 186, 187, 188, 189, 190, 191,192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227 or 228, of WO2018/226622, each of which are herein incorporated by reference, or pharmaceutically acceptable salts thereof. In one embodiment, it refers to compounds 3, 10, 14, 15, 16, 17, 18, 19, 20, 21, 22, 23, 24, 25, 26, 27, 30, 31, 32, 33, 34, 35, 36, 37, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 92, 93, 94, 95, 96, 98, 99, 100, 101, 102, 103, 104, 105, 106, 107, 108, 110, 111, 112, 113, 114, 115, 116, 117, 118, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 714, 175, 176, 177, 178, 179, 180, 181, 182, 183, 184, 185, 186, 187, 188, 189, 190, 191,192, 193, 194, 195, 196, 197, 198, 199, 200, 201, 202, 204, 205, 206, 207, 208, 209, 210, 211, 212, 213,214, 215, 216, 217, 218, 219, 220, 221, 222, 223, 224, 225, 226, 227 or 228, of WO2018/226622, each of which are herein incorporated by reference, or pharmaceutically acceptable salts thereof. In one embodiment, it refers to compounds 15, 17, 19, 20, 21, 22, 25, 26, 32, 33, 34, 35, 36, 37, 43, 45, 46, 47, 48, 49, 50, 51, 52, 55, 56, 57, 58, 59, 62, 63, 64, 66, 68, 69, 70, 71, 72, 73, 74, 75, 76, 77, 78, 79, 80, 81, 82, 83, 84, 86, 87, 88, 89, 90, 92, 93, 95, 96, 99, 100, 101, 102, 103, 104, 105, 106, 107, 108, 110, 111, 112, 113,114, 115, 117, 121, 122, 123, 124, 125, 126, 127, 128, 129, 130, 131, 132, 133, 134, 135, 136, 137, 139, 140, 141, 143, 144, 145, 146, 148, 149, 150, 151, 152, 153, 154, 155, 156, 157, 158, 159, 160, 161, 162, 163, 164, 165, 166, 167, 169, 171, 172, 173, 714, 175, 176, 177, 178, 179, 180, 181, 182, 183, 184, 185, 186, 187, 188, 189, 190, 192, 193, 195, 196, 197, 199, 200, 201, 202, 204, 206, 207, 210, 211, 215, 216, 217, 218, 219, 221, 223, 224, 225, 226 or 227, of WO2018/226622, each of which are herein incorporated by reference, or pharmaceutically acceptable salts thereof. In another embodiment, it refers to one or more compounds selected from claim 6 of WO2018/226622, each of which are herein incorporated by reference, or pharmaceutically acceptable salts thereof. In another embodiment, it refers to one or more compounds selected from claim 7 of WO2018/226622, each of which are herein incorporated by reference, or pharmaceutically acceptable salts thereof.

The term “List 6”, as used herein above and below [e.g. in Embodiments (A) or (B)], refers to compounds disclosed in WO2019/005980, such as compounds of the claims and Examples thereof. In particular, it refers to compounds of the Examples of WO2019/005980, which are incorporated herein by reference, such as compounds of the Examples and claims (e.g. compounds according to any one of claims 1 to 9, each of which are herein incorporated by reference), or pharmaceutically acceptable salts thereof. In one embodiment, it refers to any one of compounds 1 to 877 of WO2019/005980, each of which are herein incorporated by reference, or pharmaceutically acceptable salts thereof. In one embodiment, it refers to compounds 209, 287, 302, 305, 306, 413, 422, 452, 480, 502, 504, 516, 566, 587, 653, 654, 655, 656, 696, 710, 711, 713, 718, 719, 738, 752, 753, 756, 763, 791, 796, 864, 869, 870, 871, 872, 873, 874, 875 or 877, of WO2019/005980, each of which are herein incorporated by reference, or pharmaceutically acceptable salts thereof. In one embodiment, it refers to compounds 413, 502, 516, 653, 654, 696, 719, 791, 796, 864, 869, 870, 871, 872, 875 or 877, of WO2019/005980, each of which are herein incorporated by reference, or pharmaceutically acceptable salts thereof. In another embodiment, it refers to one or more compounds selected from claim 6 of WO2019/005980, each of which are herein incorporated by reference, or pharmaceutically acceptable salts thereof. In another embodiment, it refers to one or more compounds selected from claim 7 of WO2019/005980, each of which are herein incorporated by reference, or pharmaceutically acceptable salts thereof. In another embodiment, it refers to one or more compounds selected from claim 8 of WO2019/005980, each of which are herein incorporated by reference, or pharmaceutically acceptable salts thereof. In another embodiment, it refers to one or more compounds selected from claim 9 of WO2019/005980, each of which are herein incorporated by reference, or pharmaceutically acceptable salts thereof.

The term “List 7”, as used herein above and below [e.g. in Embodiments (A) or (B)], refers to compounds disclosed in WO2019/005993, such as compounds of the claims and Examples thereof. In particular, it refers to compounds of the Examples of WO2019/005993, which are incorporated herein by reference, such as compounds of the Examples and claims (e.g. compounds according to any one of claims 1 to 4, each of which are herein incorporated by reference), or pharmaceutically acceptable salts thereof. In one embodiment, it refers to compounds 801, 802, 803, 804, 805, 806, 807, 808, 810, 811, 812, 813, 814, 815, 816, 817, 818, 821, 822, 827, 828, 835, 878, 879, 880, 881, 882, 883, 884, 885, 886, 887, 888, 889, 890, 891,892, 893, 894, 895, 896, 897, 898, 899, 900, 901, 902, 903, 904, 905, 906, 907, 908, 910, 911, 912, 913, 914, 915, 916, 917, 918, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963 or 964, of WO2019/005993, each of which are herein incorporated by reference, or pharmaceutically acceptable salts thereof. In one embodiment, it refers to compounds 886 or 899 of WO2019/005993, each of which are herein incorporated by reference, or pharmaceutically acceptable salts thereof. In one embodiment, it refers to compound 954 of WO2019/005993, which is herein incorporated by reference. In another embodiment, it refers to one or more compounds selected from claim 4 of WO2019/005993, each of which are herein incorporated by reference, or pharmaceutically acceptable salts thereof.

The term “List 8”, as used herein above and below [e.g. in Embodiments (A) or (B)], refers to compounds disclosed in WO2020/005873, which are incorporated herein by reference, such as compounds of the Examples and claims (e.g. compounds according to any one of claims 1 to 7, each of which are herein incorporated by reference, for example, taken as such or in a combination thereof), or pharmaceutically acceptable salts thereof. In one embodiment, it refers to any one, such as one or more, of compounds 1 to 176 of WO2020/005873, or pharmaceutically acceptable salts thereof, each of which are herein incorporated by reference, or pharmaceutically acceptable salts thereof.

The term “List 9”, as used herein above and below [e.g. in Embodiments (A) or (B)], refers to compounds disclosed in WO2020/005877, which are incorporated herein by reference, such as compounds of the Examples and claims (e.g. compounds according to any one of claims 1 to 7, each of which are herein incorporated by reference, for example, taken as such or in a combination thereof), or pharmaceutically acceptable salts thereof. In one embodiment, it refers to any one, such as one or more, of compounds 1 to 399 of WO2020/005877, or pharmaceutically acceptable salts thereof, each of which are herein incorporated by reference, or pharmaceutically acceptable salts thereof.

The term “List 10”, as used herein above and below [e.g. in Embodiments (A) or (B)], refers to compounds disclosed in WO2020/005882, which are incorporated herein by reference, such as compounds of the Examples and claims (e.g. compounds according to any one of claims 1 to 6, each of which are herein incorporated by reference, for example, taken as such or in a combination thereof), or pharmaceutically acceptable salts thereof. In one embodiment, it refers to any one, such as one or more, of compounds 1 to 226 of WO2020/005882, or pharmaceutically acceptable salts thereof, each of which are herein incorporated by reference, or pharmaceutically acceptable salts thereof.

The term “List 11”, as used herein above and below [e.g. in Embodiments (A) or (B)], refers to compounds disclosed in WO2019/191092, which are incorporated herein by reference, such as compounds of the Examples and claims (e.g. compounds according to any one of claims 1 to 9, each of which are herein incorporated by reference, for example, taken as such or in a combination thereof), or pharmaceutically acceptable salts thereof. In one embodiment, it refers to any one, such as one or more, of compounds 1 to 65 of WO2019/191092, or pharmaceutically acceptable salts thereof, each of which are herein incorporated by reference, or pharmaceutically acceptable salts thereof.

The term “List 12”, as used herein above and below [e.g. in Embodiments (A) or (B)], refers to compounds disclosed in WO2018/0232039, which are incorporated herein by reference, such as compounds of the Examples and claims (e.g. compounds according to claim 1, which are herein incorporated by reference), or pharmaceutically acceptable salts thereof. In one embodiment, it refers to any one, such as one or more, of compounds 1 to 465 of WO2018/0232039, or pharmaceutically acceptable salts thereof, each of which are herein incorporated by reference, or pharmaceutically acceptable salts thereof.

The term “List 13”, as used herein above and below [e.g. in Embodiments (A) or (B)], refers to compounds disclosed in WO2019/028440, which are incorporated herein by reference, such as compounds of the Examples and claims (e.g. compounds according to any one of claims 1 to 43 and 170, each of which are herein incorporated by reference, for example, taken as such or in a combination thereof), or pharmaceutically acceptable salts thereof. In one embodiment, it refers to any one, such as one or more, of compounds 1 to 357 in Table 1A, Table 1B and Table 1C of WO2019/028440, or pharmaceutically acceptable salts thereof, each of which are herein incorporated by reference, or pharmaceutically acceptable salts thereof.

The term “List 14”, as used herein above and below [e.g. in Embodiments (A) or (B)], refers to compounds disclosed in WO2020/163544, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 3 and Table 5 of WO2020/163544, each of which are herein incorporated by reference, or pharmaceutically acceptable salts thereof. In one embodiment, it refers to any one, such as one or more, of compounds in Table 1A, Table 1B, Table 1C, Table 1D, Table 1E, Table 1F, Table 1G, Table 1H, Table 11 and Table 1J of WO2020/163544, each of which are herein incorporated by reference, or pharmaceutically acceptable salts thereof.

The term “List 15”, as used herein above and below [e.g. in Embodiments (A) or (B)], refers to compounds disclosed in WO2020/163401, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1A, Table 1B and Table 1C of WO2020/163401, each of which are herein incorporated by reference, or pharmaceutically acceptable salts thereof.

The term “List 16”, as used herein above and below [e.g. in Embodiments (A) or (B)], refers to compounds disclosed in WO2020/163405, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 3, Table 4, Table 5, Table 6, and Table 7 of WO2020/163405, each of which are herein incorporated by reference, or pharmaceutically acceptable salts thereof. In one embodiment, it refers to any one, such as one or more, of compounds in Table 1A, Table 1B, Table 1C, Table 1D, Table 1E, Table 1F, Table 1G, Table 1H, Table 1J, Table 1K, Table 1L, and Table 1M of WO2020/163405, each of which are herein incorporated by reference, or pharmaceutically acceptable salts thereof.

The term “List 17”, as used herein above and below [e.g. in Embodiments (A) or (B)], refers to compounds disclosed in WO2020/163409, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1A, Table 1B, Table 1C, Table 1D, Table 1E and Table 1F of WO2020/163409, each of which are herein incorporated by reference, or pharmaceutically acceptable salts thereof.

The term “List 18”, as used herein above and below [e.g. in Embodiments (A) or (B)], refers to compounds disclosed in WO2020/163323, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1A, Table 1B, Table 1C, Table 1D, Table 1E, Table 1F, Table 1G, Table 1H, Table 11, Table 1J, Table 1K and Table 1L of WO2020/163323, each of which are herein incorporated by reference, or pharmaceutically acceptable salts thereof. In one embodiment, it refers to any one, such as one or more, of compounds in Table 3, Table 4, and Table 5 of WO2020/163323, each of which are herein incorporated by reference, or pharmaceutically acceptable salts thereof.

The term “List 19”, as used herein above and below [e.g. in Embodiments (A) or (B)], refers to compounds disclosed in WO2020/163375, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1, Table 2, Table 3, Table 4, Table 5, Table 6, Table 7, Table 8, Table 9, Table 10, Table 11, Table 12, Table 13, Table 14, Table 15, Table 16, Table 17, Table 18, Table 19, Table 20, Table 21, Table 22 and Table 23 of WO2020/163375, each of which are herein incorporated by reference, or pharmaceutically acceptable salts thereof. In one embodiment, it refers to any one, such as one or more, of compounds in Table A-10, Table A-12 and Table 24 of WO2020/163375, each of which are herein incorporated by reference, or pharmaceutically acceptable salts thereof.

The term “List 20”, as used herein above and below [e.g. in Embodiments (A) or (B)], refers to compounds disclosed in WO2020/163406, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1 of WO2020/163406, each of which are herein incorporated by reference, or pharmaceutically acceptable salts thereof.

The term “List 21”, as used herein above and below [e.g. in Embodiments (A) or (B)], refers to compounds disclosed in WO2020/163541, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1A, Table 1C, Table 1E, Table 1G and Table 1H of WO2020/163541, each of which are herein incorporated by reference, or pharmaceutically acceptable salts thereof. In one embodiment, it refers to any one, such as one or more, of compounds in Table 15 of WO2020/163541, each of which are herein incorporated by reference, or pharmaceutically acceptable salts thereof.

The term “List 22”, as used herein above and below [e.g. in Embodiments (A) or (B)], refers to compounds disclosed in WO2020/163647, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1, Table 2 and Table 5 of WO2020/163647, each of which are herein incorporated by reference, or pharmaceutically acceptable salts thereof. In one embodiment, it refers to any one, such as one or more, of compounds in Table B, Table C, Table A-5 and Table A-6 of WO2020/163647, each of which are herein incorporated by reference, or pharmaceutically acceptable salts thereof.

The term “List 23”, as used herein above and below [e.g. in Embodiments (A) or (B)], refers to compounds disclosed in WO2020/163248, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1A, Table 1B, Table 1C, Table 1D, Table 1E, Table 1F, Table 1G, Table 1H, Table 11 and Table 1J of WO2020/163248, each of which are herein incorporated by reference, or pharmaceutically acceptable salts thereof. In one embodiment, it refers to any one, such as one or more, of compounds in Table 3B, Table 4, Table 6, Table 7, Table 8 and Table 9 of WO2020/163248, each of which are herein incorporated by reference, or pharmaceutically acceptable salts thereof.

The term “List 24”, as used herein above and below [e.g. in Embodiments (A) or (B)], refers to compounds disclosed in WO2020/163382, which are incorporated herein by reference, such as compounds of the Examples, tables and claims (e.g. compounds according to any one of claims, each of which are herein incorporated by reference, for example, taken as such or in a combination thereof), or pharmaceutically acceptable salts thereof. In one embodiment, it refers to any one, such as one or more, of compounds in Table 1A and Table 1B of WO2020/163382, each of which are herein incorporated by reference, or pharmaceutically acceptable salts thereof.

As used herein, the compound named branaplam, as used herein above and below, is the splicing modulator also named 5-(1H-Pyrazol-4-yl)-2-(6-((2,2,6,6-tetramethylpiperidin-4-yl)oxy)pyridazin-3-yl)phenol, of formula (I):

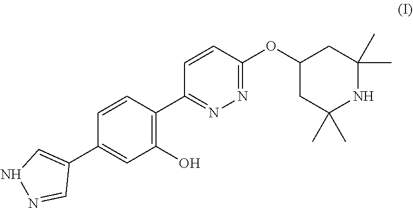 [SMILES: CC1(C)CC(Oc2ccc(-c3ccc(-c4cn[nH]c4)cc3O)nn2)CC(C)(C)N1]

Branaplam, or pharmaceutical salt thereof, such as branaplam hydrochloride salt, can be prepared as described in WO2014/028459, which is incorporated herein by reference, e.g. in Example 17-13 therein. As used herein, “branaplam” refers to the free form, and any reference to “a pharmaceutically acceptable salt thereof” refers to a pharmaceutically acceptable acid addition salt thereof. As used herein, the term “branaplam, or a salt thereof, such as a pharmaceutically acceptable salt thereof”, as used in the context of the present invention (especially in the context of the any of the embodiments, above or below, and the claims) is thus to be construed to cover both the free form and a pharmaceutically acceptable salt thereof, unless otherwise indicated herein. As used herein, the term “branaplam hydrochloride salt” or “branaplam monohydrochloride salt” refers to 5-(1H-Pyrazol-4-yl)-2-(6-((2,2,6,6-tetramethylpiperidin-4-yl)oxy)pyridazin-3-yl)phenol monohydrochloride salt or hydrate thereof, such as 5-(1H-Pyrazol-4-yl)-2-(6-((2,2,6,6-tetramethylpiperidin-4-yl)oxy)pyridazin-3-yl)phenol monohydrochloride monohydrate, also named branaplam hydrochloride monohydrate. In particular, branaplam is in the form of branaplam hydrochloride salt.

In one embodiment, the term “splicing modulator”, as used herein, refers to a small molecule that directly or indirectly increases association of a target pre-mRNA sequence with the spliceosome to enhance or reduce gene expression.

In one embodiment, the term “splicing modulator”, as used herein, refers to a compound, e.g., a small molecule, that alters splicing of a precursor messenger RNA (abbreviated as pre-mRNA). Exemplary splicing modulators alter the recognition of splice sites by the spliceosome, e.g., by interacting with components of the splicing machinery (e.g. the proteins and/or the nucleic acids (e.g., mRNAs and/or pre-mRNAs)), which leads to an alteration of normal splicing of the targeted pre-mRNA. Exemplary splicing modulators thus alter the sequence (or relative level of one or more sequences) of a mature RNA product of a targeted pre-mRNA. Exemplary splice modulators act by directly or indirectly altering, e.g., increasing, association of a target pre-mRNA sequence with the spliceosome to, e.g., enhance or reduce gene expression. Non-limiting examples of splicing modulators are small molecules (e.g. branaplam) and oligonucleotides, such as antisense oligonucleotides and splice-switching oligonucleotides (SSOs). More examples of splicing modulators can be found e.g. in WO2014/028459, WO2014/116845 and WO2015/017589, which are incorporated herein by reference in their entirety, or in WO2020/005873, WO2020/005877, WO2020/005882 and WO2019/191092, which are incorporated herein by reference in their entirety. Certain oligomeric compounds and nucleobase sequences that may be used to alter splicing of a pre-mRNA may be found for example in U.S. Pat. Nos. 6,210,892; 5,627,274; 5,665,593; 5,916,808; 5,976,879; US2006/0172962; US2007/002390; US2005/0074801; US2007/0105807; US2005/0054836; WO 2007/090073; WO2007/047913, Hua et al., PLOS Biol 5(4): e73; Vickers et al., J. Immunol. 2006 Mar. 15; 176(6):3652-61; and Hua et al., American J. of Human Genetics (April 2008) 82, 1-15, each of which is hereby incorporated by reference in its entirety for any purpose. Antisense compounds have also been used to alter the ratio of naturally-occurring alternative splice variants such as the long and short forms of Bcl-X pre-mRNA (U.S. Pat. No. 6,172,216: U.S. Pat. No. 6,214,986: Taylor et al., Nat. Biotechnol. 1999, 17, 1097-1100) or to force skipping of specific exons containing premature termination codons (Wilton et al., Neuromuscul. Disord., 1999, 9, 330-338). U.S. Pat. No. 5,627,274 and WO 94/26887 disclose compositions and methods for combating aberrant splicing in a pre-mRNA molecule comprising a mutation using antisense oligonucleotides which do not activate RNAse H.

In one embodiment, the relative expression level of a naturally-occurring alternative splice variant is altered, e.g. the ratio of one splice variant derived from a target pre-mRNA is changed with respect to another splice variant or the whole pool of splice variants derived from that pre-mRNA.

In another embodiment, a new splice variant is generated while the ratio of naturally-occurring alternative splice variants may or may not be altered. In one embodiment, said new splice variant is generated by removal of one or more nucleic acids from the mRNA otherwise produced in the absence of the splice modulator. This may occur, for example, by exon skipping, i.e. wherein an exon is spliced out of the pre-mRNA and is therefore not present in the mature mRNA. In another embodiment, said new splice variant is generated by activation of an alternative donor site, where an alternative 5′ splice junction (donor site) is used, changing the 3′ boundary of the upstream exon. In another embodiment, said new splice variant is generated by activation of an alternative acceptor site, where an alternative 3′ splice junction (acceptor site) is used, changing the 5′ boundary of the downstream exon. In a preferred embodiment, said new splice variant is generated which includes additional sequence not included in the mRNA in the absence of the splice modulator, e.g., by intron retention, where additional sequence, e.g., an intron or a portion thereof, is retained in the pre-mRNA and therefore is included in the mature mRNA. Therefore, if the retained sequence, e.g., intron or portion thereof, is in the coding region, said intron retention may lead to generation of,
a) a splice variant encoding additional amino acids encoded by the retained intron (for example, in the case that said intron does not cause a frameshift and does not introduce a stop codon in the reading frame), or, in an embodiment,
b) a splice variant containing a premature stop codon, e.g. inclusion of the additional sequence, e.g., the intron or portion thereof, causes a frameshift and/or introduces sequence comprising an in-frame stop codon upstream of the original stop codon, and therefore the resulting splice variant mRNA encodes a protein lacking one or more amino acid residues, e.g., in the C-terminus, compared to the protein encoded by a splice variant in which said intron-retention has not taken place. In a preferred embodiment, the expression level of the encoded protein is altered, e.g., is reduced, in the presence of the splice modulator relative to the expression level of the protein encoded by the splice variant in the absence of the splice modulator. In a preferred embodiment, the expression level of the splice variant (transcript) is less than the expression level of a splice variant without the intron retention. In particular, said reduced expression levels is at least partly due to instability (e.g. reduced half-life) and/or increased degradation of the resulting mRNA or encoded polypeptide, for example via a nonsense-mediated decay mechanism (in the case of the mRNA) or increased protein degradation (in the case of the encoded polypeptide).

A “splice variant” as the term is used herein refers to a mature mRNA species that is produced from a particular pre-mRNA, or a polypeptide encoded by said mature mRNA species. A particular pre-mRNA species of interest may produce one or more splice variants.

In one embodiment, a splicing modulator is a SMN splicing modulator, for example a SMN2 splicing modulator. In a preferred embodiment, the splicing modulator according to the present invention modulates splicing of the HTT gene between exons 49 and 50.

The term “SMN splicing modulator” (e.g. SMN2 splicing modulator) refers to a compound (e.g. a small molecule) that directly or indirectly increases association of the SMN2 pre-mRNA sequence with the spliceosome to enhance SMN2 exon7 inclusion and increase SMN expression.

The term “the splicing modulator is provided in the form of a pharmaceutical composition”, as used herein, refers to a pharmaceutical composition comprising the splicing modulator and at least one pharmaceutically acceptable excipient.

The term “the splicing modulator is provided in the form of a pharmaceutical combination”, as used herein, refers to a pharmaceutical combination comprising the splicing modulator and at least one further pharmaceutical active ingredient.

An “antisense compound” as used herein refers to a compound (e.g., an antisense oligonucleotide) that hybridizes (e.g., via base pairing) to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Examples of antisense compounds that target Huntington's disease are described in, for example, WO19157531, WO18022473, WO17015575, WO17192664, WO15107425, WO14121287, WO14059356, WO14059341, WO13033223, WO12109395, WO13022990, WO12012467, WO11097643, WO11097644, WO11097641, WO11032045, WO07089584, WO07089611, the contents of which are hereby incorporated by reference in their entirety. Additional examples of antisense compounds that target Huntington's disease include RG6042 (Roche), WVE-120101 (Wave/Takeda) and WVE-120102 (Wave/Takeda).

The term gene therapy, as used herein, refers, for example, to AMT-130, described, for example, in WO 2016/102664, which is hereby incorporated by reference in its entirety.

The term “pharmaceutical composition” is defined herein to refer, for example, to a mixture or solution containing at least one active ingredient or therapeutic agent to be administered to a subject, in order to treat a subject, for example as herein defined. The compounds specified herein [e.g. a splicing modulator selected from the group consisting of List 1, List 2, List 3, List 4, List 5, List 6, List 7, List 8, List 9, List 10, List 11, List 12 and List 13, such as List 1, List 2, List 3, List 4, List 5, List 6 and List 7; e.g. branaplam] can be administered by conventional route, in particular orally, which can be manufactured according to pharmaceutical techniques as known in the art (for example in “Remington Essentials of Pharmaceutics, 2013, 1^{st} Edition, edited by Linda Felton, published by Pharmaceutical Press 2012, ISBN 978 0 85711 105 0; in particular Chapter 30), wherein pharmaceutical excipients are, for example, as described in Handbook of Pharmaceutical Excipients, 2012, 7^{th} Edition, edited by Raymond C. Rowe, Paul J. Sheskey, Walter G. Cook and Marian E. Fenton, ISBN 978 0 85711 027 5.

As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.

For the above-mentioned uses/treatment methods the appropriate dosage may vary depending upon a variety of factors, such as, for example, the age, weight, sex, the route of administration or salt employed.

As used herein, the term “a,” “an,” “the” and similar terms used in the context of the present invention (especially in the context of the embodiments and claims) are to be construed to cover both the singular and plural unless otherwise indicated herein or clearly contradicted by the context.

The use of any and all examples, or exemplary language (e.g. “such as”) provided herein is intended merely to better illuminate the invention and does not pose a limitation on the scope of the invention otherwise claimed.

The term “compound of the present invention”, as used herein, refers to a “splicing modulator”, as defined herein, and is understood to be in free form or in the form of a pharmaceutically acceptable salt.

As used herein, the terms “free form” or “free forms” refers to the compound in non-salt form, such as the base free form or the acid free form of a respective compound, e.g. the compounds specified herein [e.g. selected from the group consisting of List 1, List 2, List 3, List 4, List 5, List 6, List 7, List 8, List 9, List 10, List 11, List 12 and List 13, in particular List 1, List 2, List 3, List 4, List 5, List 6 and List 7, as defined herein].

As used herein, the terms “salt”, “salts” or “salt form” refers to an acid addition or base addition salt of a respective compound, e.g. the compounds specified herein [e.g. selected from the group consisting of List 1, List 2, List 3, List 4, List 5, List 6, List 7, List 8, List 9, List 10, List 11, List 12 and List 13, in particular List 1, List 2, List 3, List 4, List 5, List 6 and List 7, as defined herein]. “Salts” include in particular “pharmaceutically acceptable salts”. The term “pharmaceutically acceptable salts” refers to salts that retain the biological effectiveness and properties of the compounds and, which typically are not biologically or otherwise undesirable.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).

The terms “drug”, “active substance”, “active ingredient”, “pharmaceutically active ingredient”, “active agent”, “therapeutic agent” or “agent” are to be understood as meaning a compound in free form or in the form of a pharmaceutically acceptable salt.

The term “combination” or “pharmaceutical combination” refers to either a fixed combination in one unit dosage form, non-fixed combination, or a kit of parts for the combined administration where a compound of the present invention and one or more combination partner (e.g. another drug, also referred to as further “pharmaceutical active ingredient”, “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. the compound of the present invention and one or more combination partners, are both administered to a patient simultaneously in the form of a single entity or dosage. The term “non-fixed combination” means that the active ingredients, e.g. a compound of the present invention and one or more combination partners, are both administered to a patient as separate entities either simultaneously or sequentially with no specific time limits, wherein such administration provides therapeutically effective levels of the two compounds in the body of the patient.

The compound of the present invention may be administered separately, by the same or different route of administration, or together in the same pharmaceutical composition as the other agents. In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.

### ABBREVIATIONS

h=hr=hour(s)
min=minute(s)
sec=second(s)
msec=millisecond(s)
mg=milligram(s)
Kg=kg=kilogram(s)
ml=mL=milliliter(s)
uL==ul=microliter(s)
PCR=polymerase chain reaction
rpm=revolutions per minute
° C.=degree(s) Celsius
xg=times gravity (centrifugal force)
HTT=Huntingtin
mHTT=mutant Huntingtin
tHTT=total Huntingtin
HTRF=Homogenous Time Resolved Fluorescence
n=number of animals
CSF=cerebrospinal fluid
cP=centipoise, unit of viscosity
pIC₅₀=−Log(IC₅₀) where IC₅₀ is expressed in molar or mol/L
RT=room temperature (25±3° C.)
56-FAM=5′ 6-FAM (Fluorescein)
3IABKFQ=3′ Iowa Black® FAM quencher
ZEN=ZEN™ internal quencher
USA=United States of America
BacHD=BACH=Bacterial artificial chromosome-mediated transgenic Huntington's Disease
model
ACAT=Advanced Compartmental And Transit
AUC=area under the curve
BIW=twice per week
CD=cyclodextrin
CL=clearance of elimination from central compartment
Cmax=maximum concentration
Ctrough=minimum concentration
F=bioavailability
Fa=fraction absorbed
IC50=half-maximum inhibitory concentration
Imax: maximum inhibition effect
k12=first-order rate constant from compartment 1 to compartment 2
k21=first-order rate constant from compartment 2 to compartment 1
ka=first-order absorption rate constant
kin=synthesis rate of mutant HTT protein
kout=degradation rate or fractional turnover parameter of the mutant HTT protein synthesis
Log P=logarithm of partition coefficient between organic and aqueous solution
MTD=maximum tolerated dose
PBPK=Physiologically Based Pharmacokinetic (PBPK)
PD=pharmacodynamics
Peff=effective permeability
PK=pharmacokinetics
pka=negative logarithm of the acid dissociation constant
Q=intercompartmental clearance
QW=once per week
R=mutant HTT protein level at a given time
RO=baseline of mutant HTT protein concentration (e.g. in brain)
RSE=relative standard error reported on the approximate standard deviation scale
(SE/variance)/2
SE=standard deviation scale
ss=steady state
SMA=spinal muscular atrophy
Tlag=lag time of absorption
Tmax=time of maximum concentration
V1=central volume of 2-compartment PK model
V2, Vc=peripheral volume of 2-compartment PK model
T½=apparent terminal elimination half life

### EXAMPLES

### Example 1a: Pre-Clinical Evaluation of Branaplam

### Methods

RNA-Seq Analysis of Human Cells Treated with Splice Modulators

A normal Human Fibroblast line (HD1994) was treated with branaplam and Splice modulator 2 (described as Example 3-2 in WO 2015017589) and Splice modulator 3 (described as NVS-SM3 in Nat Chem Biol. 2015 July; 11(7):511-7. doi: 10.1038/nchembio.1837) or DMSO for 24 hours. The following compound doses were used:
Branaplam was used at an efficacious dose (100 nM) and a cytotoxic dose (5 uM).
Splice modulator 2 was used at 750 nM.
Splice modulator 3 was used at 5 uM.

There were 3 biological replicates per group.

Total RNA was isolated using the Qiagen RNeasy Mini isolation kit. RNA-Seq libraries were prepared using the Illumina TruSeq RNA Sample Prep kit v2 and sequenced using the Illumina HiSeq 2500 platform.

Each sample was sequenced on four different lanes belonging to the same flow cells to a length of 2×76 base-pairs (bp). The quality of the generated reads was assessed by running FastQC (version 0.10.1) on the FASTQ files. The average quality per base in Phred score was computed for each sample. The reads were of excellent quality (mean Phred score >28 for all base positions). A total of 847 million 76-base-pair (bp) paired-end reads were mapped to the Homo sapiens genome (hg19), the human RefSeq (Pruitt et al., 2007) transcripts (release 59, May 3, 2013) using TopHat (2.0.3).

In order to increase the ability to detect exons, the three alignment files (bam files) for each of the five conditions (DMSO, branaplam at 5 uM, branaplam at 100 nM, splice modulator 2 at 500 nM and splice modulator 3 at 5 uM) were pooled before the transcript assembly by Cufflinks (2.1.1). After transcript assembly, the exon coordinates were extracted from the transcript gtf files. Exons on alternative chromosomes and on chromosome M were excluded and the strand information were ignored. That yielded 273866 putative exons. Exons that do not intersect any RefSeq exon (release 59, May 3, 2013) were considered as candidates for non-annotated splice in events. That resulted in 19474 candidates. To gain further confidence, we merged overlapping exons in the full set of all RefSeq exons plus the initial 19474 candidates resulting in 229665 non-overlapping exons. For this set of exons, all possible exon-exon junctions within each RefSeq gene were considered. A junction database was created using R (2.15.2) scripts and bedtools (2.15.0). The first mate of each paired end read was then mapped against the database. Only non-annotated exons supported by at least one junction alignment were retained. This excludes in particular candidates not attached to a RefSeq gene. That left 10898 final candidates. Sequences for these candidates were extracted from hg19 using bedtools. To assess variability, separate Cufflinks assemblies for each replicate were also computed and presence of each candidate in such an assembly was checked. In addition, the alignments against the junction database were used to determine the number of junctions that skip over a novel exon. That information was used to estimate a splice in fraction. Further, the read coverage for the 10898 candidates was determined for each replicate using bedtools on the TopHat alignments (bam files) and then aggregated within each of the five conditions. The original fastq files were reprocessed with STAR (020201) and aligned against the human genome (hg38). The 10898 candidate exons were lifted over to hg38 using the UCSC genome browser tools—7 candidates could not be lifted. The junctions detected by STAR were mapped to the remaining 10891 candidates and provide an alternative source of junction counts.

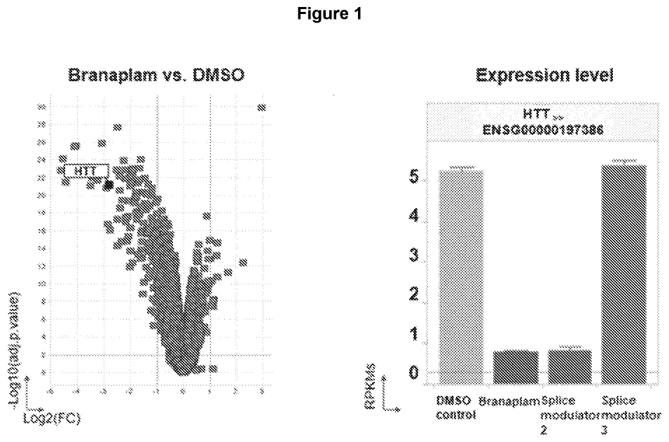

A candidate, falling into the gene region of HTT (chr4:3213622-3213736) was detected and appeared to be modulated by active compounds. It was supported by the re analysis with STAR alignments. In addition, the 3′ end shows the AGA|GT motif that is associated to the mode of action of the active compounds. Finally, this candidate (comprised in the splice variant herein referred to as the novel-exon-containing HTT transcript) could be verified by PCR as described below. The candidate chr4:3213622-3213736 introduces an in-frame stop codon (TAG) which is 55 nucleotides from the 3′ end of the exon and therefore may trigger nonsense-mediated decay. NGS data show that expression of HTT is downregulated by the active compounds about six fold (FIG. 1). A partial sequence (showing only the part corresponding to exon 49, novel exon, and exon 50) of the novel-exon-containing HTT transcript is included herein as SEQ ID NO: 9. The novel exon is underlined.

SEQ ID NO: 9
GGGATGCTGCACTGTATCAGTCCCTGCCCACTCTGGCCCGGGCCCTGG
CACAGTACCTGGTGGTGGTCTCCAAACTGCCCAGTCATTTGCACCTTC
CTCCTGAGAAAGAGAAGGACATTGTGAAATTCGTGGTGGCAACCCTTG
AGAGGCAAGCCCTGGTGCTGTGGGAGCCCCAAGGAAGAGCCTCTGGCC
TGGTGGCCACGTAGCCCAGGAGAGATTTCTACAGGAGCCCACAGCGCT
GAAGGAGAGAGAGGCAGCAGAGCCCTGTCCTGGCATTTGATCCATGAG
CAGATCCCGCTGAGTCTGGATCTCCAGGCAGGGCTGGACTGCTGCTGC
CTGGCCCTGCAGCTGCCTGGCCTCTGGAGCGTGGTCTCCTCCACAGAG
TTTGTGACCCACGCCTGCTCCCTCATCTACTGTGTGCACTTCATCCTG
GAGGCCG

### In Vitro Evaluation of Branaplam

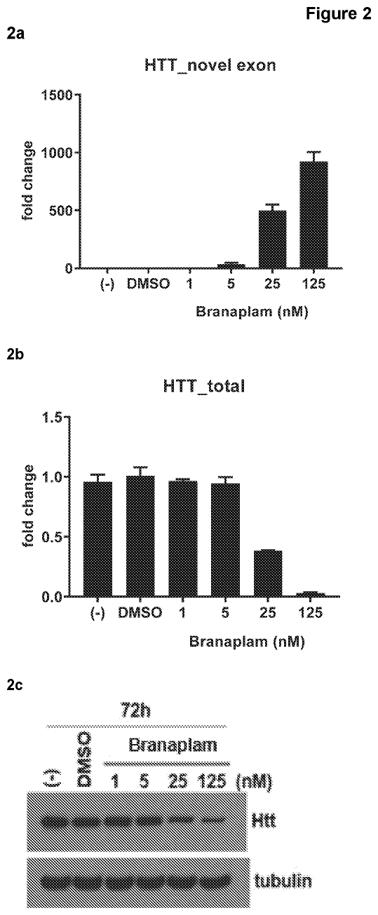

Cultured human neuroblastoma (SH-SY5Y cell line) cells were treated at doses ranging from 5 nM-125 nM for 24 hours (for transcript evaluation) or 48 hours (for protein evaluation). RNA was quantified by Nanodrop 2000 (Thermo Scientific). cDNAs were synthesized from 140-400 ng RNA using Maxima First strand cDNA synthesis kit using a mix of oligo dT and random hexamers (Thermo Scientific) in 20 uL reaction at 25° C. for 10 min, 50° C. for 15 min then 85° C. for 5 min. Quantitative PCR was performed using Taqman Fast Advanced master mix (Thermo Scientific) in 20 uL with 4 uL of cDNA reaction and primers specific for each genes. The PCR steps were as follows: 95° C. for 20 sec then 40 cycles of 95° C. for 1 sec, 55° C. for 20 sec. The sequence of primers were, for WT human HTT, forward, 5′-GTCATTTGCACCTTCCTCCT-3′ (SEQ ID NO: 1); reverse, 5′-TGGATCAAATGCCAGGACAG-3′ (SEQ ID NO: 2) and sequence of probe was 56-FAM/TTG TGA AAT/ZEN/TCG TGG TGG CAA CCC/3IABKFQ/(SEQ ID NO: 8), for HTT novel exon, forward, 5′-TCCTGAGAAAGAGAAGGACATTG-3′ (SEQ ID NO: 3); reverse, 5′-CTGTGGGCTCCTGTAGAAATC-3′ (SEQ ID NO: 4) and sequence of probe/56-FAM/AAT TCG TGG/ZEN/TGG CAA CCC TTG AGA/3IABKFQ/(SEQ ID NO: 7). Relative quantification of gene expression was performed using 2-AACT method. Fold changes in the mRNA expression level was calculated following normalization to mouse glucuronidase beta (Gusb) as an endogenous reference (FIGS. 2a and 2b).

For protein analysis, cells were lysed in RIPA buffer with protease and phosphatase inhibitor (Thermo Scientific). Supernatant was obtained by centrifugation for 20 min at 13,000 rpm at 4° C. Total protein concentration was quantified using the BCA protein Assay (Thermo Scientific). Samples were resolved in 3-8% Tris-Acetate protein gel under reducing condition. Proteins were transferred onto PVDF membrane (Millipore) and western blot analysis was performed using rabbit anti-Huntingtin antibody (Millipore #MAB2166), mouse anti-actin (Sigma, #A5316) and mouse anti-vinculin (Bio-Rad, #MCA465). Protein bands were quantified by Image J.

### BacHD Mice

Twenty-eight BacHD mice (FVB/N-Tg(HTT*97Q)|Xwy/J transgenic mice—Jackson Laboratories) were used for the experiment. Animal protocols were approved by the Children's Hospital of Philadelphia Institutional Animal Care and use Committee. Mice were housed in a temperature-controlled environment on a 12-h light/dark cycle. Food and water were provided ad libitum.

For the repeat dosing study, twenty-four BacHD mice (FVB/N-Tg(HTT*97Q)IXwy/J transgenic mice—Jackson Laboratories) were used for the experiment. Animal protocols were approved by the Children's Hospital of Philadelphia Institutional Animal Care and use Committee. Mice were housed in a temperature-controlled environment on a 12-h light/dark cycle. Food and water were provided ad libitum.

### Branaplam Treatment

A single dose of branaplam or vehicle solution was administered by oral gavage. Mice were divided in seven groups (n=4 mice/group) and treated with branaplam (10 mg/kg—2 groups) and 50 mg/kg—3 groups), or vehicle (2 groups) as control. Mice were firmly restrained by grasping the loose skin to immobilize the head, maintained in a vertical position and a 22- to 26-gauge gavage needle was placed in the side of the mouth. The needle was guided following the roof of the mouth into the esophagus and allowed to gently enter in the stomach. The amount of branaplam or vehicle administrated to each mouse was based on the weight recorded before treatment.

For the repeat dosing study, branaplam or vehicle was administered by oral gavage 3 times a week for 3 consecutive weeks. Mice were divided in 4 groups (n=6 mice/group) and treated with branaplam (12 mg/kg—1 group, or 24 mg/kg—2 groups), or vehicle (1 groups) as control. Mice were firmly restrained by grasping the loose skin to immobilize the head, maintained in a vertical position and a 22- to 26-gauge gavage needle was placed in the side of the mouth. The needle was guided following the roof of the mouth into the esophagus and allowed to gently enter in the stomach. The amount of branaplam or vehicle administrated to each mouse was based on the weight recorded the day of dosing.

### Blood Collection and Tissue Sampling

At 8 h, 24 h (vehicle and branaplam 10 mg/kg & 50 mg/kg), and 48 h (branaplam 50 mg/kg) after oral gavage, blood and tissue samples were obtained for PK and PD analysis. Mice were anesthetized with isoflurane and blood was obtained via submandibular vein bleeds and collected for RNA extraction (PD analysis) using RNAprotect Animal Blood Tubes, and plasma (PK analysis) using K₂EDTA coated tubes. Cells were removed from plasma by centrifugation for 10 min at 2000×g at 4° C., and plasma samples were stored at −80° C. Following blood collection, mice were anesthetized with a lethal dose of ketamine/xylazine (100 ml of a 10 mg:1 mg), and perfused with 18 ml of 0.9% cold saline mixed with 2 ml of RNAlater (Ambion) solution for tissue collection. Liver, skeletal muscle, cerebrum, and cerebellum samples were flash frozen in liquid nitrogen and stored at −80° C.

### Blood and CSF Collection, and Tissue Sampling for Repeat Dose Study

At 24 h (vehicle and branaplam 12 mg/Kg & 24 mg/Kg), and 72 h (branaplam 24 mg/Kg) after the last treatment, blood, CSF, and tissue samples were obtained for PK and PD analysis. To collect CSF, mice were placed in a rodent anesthesia induction chamber where they are exposed to 4-5% isoflurane in 100% oxygen carrier gas. Once an appropriate plane of anesthesia was achieved, they were moved to a nose cone so that maintenance levels of isoflurane (1-3%) could be delivered throughout the procedure. The dorsal aspect of their cervical and occipital region was surgically prepped to visualize the dura mater under a microscope. A glass micropipette attached to a micromanipulator was introduced to the cisterna magna via a puncture through the dura mater at a point where no vasculature was visualized, and CSF was allowed to flow into the micropipette via capillary action. After approximately 15-30 minutes, the micropipette was removed from the cisterna magna, the CSF sample was transferred into Eppendorf tubes, flash frozen in liquid nitrogen, and stored at −80° C.

Following CSF collection, mice were kept under anesthesia with isoflurane. Blood was obtained via submandibular vein bleeds and collected for RNA extraction (PD analysis) using RNAprotect Animal Blood Tubes, and plasma (PK analysis) using K2EDTA coated tubes. Cells were removed from plasma by centrifugation for 10 min at 2000×g at 4° C., and plasma samples were stored at −80° C. Following blood collection, mice were given a lethal dose of ketamine/xylazine (100 ml of a 10 mg:1 mg), and perfused with 18 ml of 0.9% cold saline mixed with 2 ml of RNAlater (Ambion) solution for tissue collection. Liver, skeletal muscle, brain striatum, brain cortex, hemibrain and cerebellum samples were flash frozen in liquid nitrogen and stored at −80° C.

### Blood and CSF Collection, and Tissue Sampling for Repeat Dose Timecourse Study

At 72 h 168 h, 240 h and 336 h (branaplam 24 mg/kg) after the last treatment (vehicle or 24 mg/kg branaplam for 1 week or 3 weeks), blood, CSF, and tissue samples were obtained for PK and PD analysis. To collect CSF, mice were placed in a rodent anesthesia induction chamber where they are exposed to 4-5% isoflurane in 100% oxygen carrier gas. Once an appropriate plane of anesthesia was achieved, they were moved to a nose cone so that maintenance levels of isoflurane (1-3%) could be delivered throughout the procedure. The dorsal aspect of their cervical and occipital region was surgically prepped to visualize the dura mater under a microscope. A glass micropipette attached to a micromanipulator was introduced to the cisterna magna via a puncture through the dura mater at a point where no vasculature was visualized, and CSF was allowed to flow into the micropipette via capillary action. After approximately 15-30 minutes, the micropipette was removed from the cisterna magna, the CSF sample was transferred into Eppendorf tubes, flash frozen in liquid nitrogen, and stored at −80° C.

Following CSF collection, mice were kept under anesthesia with isoflurane. Blood was obtained via submandibular vein bleeds and collected for RNA extraction (PD analysis) using RNAprotect Animal Blood Tubes, and plasma (PK analysis) using K₂EDTA coated tubes. Cells were removed from plasma by centrifugation for 10 min at 2000×g at 4° C., and plasma samples were stored at −80° C. Following blood collection, mice were given a lethal dose of ketamine/xylazine (100 ml of a 10 mg:1 mg), and perfused with 18 mL of 0.9% cold saline mixed with 2 mL of RNAlater (Ambion) solution for tissue collection. Liver, skeletal muscle, brain striatum, brain cortex, hemibrain and cerebellum samples were flash frozen in liquid nitrogen and stored at −80° C.

### Branaplam Dose

In experiments, as herein described, the branaplam dose is provided as a solution of branaplam monohydrochloride salt (10 mg/mL suspension) in methyl cellulose, medium viscosity 400 cP for a 1% solution), Tween 80 (1% v/v), purified water suspension formulation.

### RNA Extraction and Real Time Quantitative PCR

Total RNA from cerebrum and cerebellum was extracted using RNeasy Plus kit (Qiagen) after homogenized in Precellys at 6000 rpm for 40 sec. RNA from blood was extracted using PAXgene blood RNA kit (Qiagen) according to manufacturer protocol. The RNA was quantified by Nanodrop 2000 (Thermo Scientific). cDNAs were synthesized from 140-400 ng RNA using Maxima First strand cDNA synthesis kit using a mix of oligo dT and random hexamers (Thermo Scientific) in 20 uL reaction at 25° C. for 10 min, 50° C. for 15 min then 85° C. for 5 min. Quantitative PCR was performed using Taqman Fast Advanced master mix (Thermo Scientific) in 20 uL with 4 uL of cDNA reaction and primers specific for each genes. The PCR steps were as follows: 95° C. for 20 sec then 40 cycles of 95° C. for 1 sec, 55° C. for 20 sec. The sequence of primers were, for WT human HTT, forward, 5′-GTCATTTGCACCTTCCTCCT-3′ (SEQ ID NO: 1); reverse, 5′-TGGATCAAATGCCAGGACAG-3′ (SEQ ID NO: 2) and sequence of probe was 56-FAM/TTG TGA AAT/ZEN/TCG TGG TGG CAA CCC/3IABKFQ/(SEQ ID NO: 8), for HTT novel exon, forward, 5′-TCCTGAGAAAGAGAAGGACATTG-3′ (SEQ ID NO: 3); reverse, 5′-CTGTGGGCTCCTGTAGAAATC-3′ (SEQ ID NO: 4) and sequence of probe/56-FAM/AAT TCG TGG/ZEN/TGG CAA CCC TTG AGA/3IABKFQ/(SEQ ID NO: 7). Relative quantification of gene expression was performed using 2^{-ΔΔ}CT method. Fold changes in the mRNA expression level was calculated following normalization to mouse glucuronidase beta (Gusb) as an endogenous reference.

### Protein Preparation and Western Blot Analysis

Snap-frozen mouse tissue samples were homogenized in RIPA buffer with protease and phosphatase inhibitor (Thermo Scientific) by Precellys at 6000 rpm for 40 sec. Supernatant was obtained by centrifugation for 20 min at 13,000 rpm at 4° C. Total protein concentration was quantified using the BCA protein Assay (Thermo Scientific). Samples were resolved in 3-8% Tris-Acetate protein gel under reducing condition. Proteins were transferred onto PVDF membrane (Millipore) and western blot analysis was performed using rabbit anti-Huntingtin antibody (Millipore #MAB2166), mouse anti-actin (Sigma, #A5316) and mouse anti-vinculin (Bio-Rad, #MCA465). Protein bands were quantified by Image J.

### Protein Analysis on CSF Samples

CSF samples were clarified after a 5 minutes centrifugation at 14,000 rpm in a centrifuge at 4° C. 96-well V-bottom plate were loaded with a CSF buffer 2.5-5 uL of CSF sample. This was followed by addition of MP-2B7 (magnetic particle antibody conjugated suspension) HTT antibody diluted in Erenna assay buffer. Assay plate was incubated with shaking (600 rpm) at RT for 1 h and then put through a post-transfer wash program on BioTek-405. 20 ul/well of MW1 detection antibody was added to the assay plate. Plate was incubated with shaking (at 750 rpm) at room temperature for 1 hr. Plate was washed on BioTek-405 and after serial buffer washes, assay plate was placed on magnetic rack till all beads were pulled to the magnet (approximately 5 min), 10 uL of sample from the assay plate were transferred to a 384-well plate. The plate was left at room temperature for 30 mins and then run on the Erenna machine using a run time of 60 seconds.

### CONCLUSIONS

In vitro evaluation of branaplam in human neuroblastoma cell line (SHSY5Y) revealed that branaplam treatment leads to a dose dependent lowering of total Huntingtin transcript to 30-90% of normal endogenous levels at doses ranging from 5 nM-125 nM and a concomitant increase (100-500 fold) in a novel-exon-containing HTT transcript (FIGS. 2a and 2b). Furthermore, Western blot analysis revealed that this decrease in transcript was accompanied by a robust reduction of normal Huntingtin protein (50-70%) in the same dose range (FIG. 2c). EC50 for lowering of HTT transcript by Branaplam was in the 20-25 nM range while EC50 for HTT protein lowering was in the 10-25 nM range.

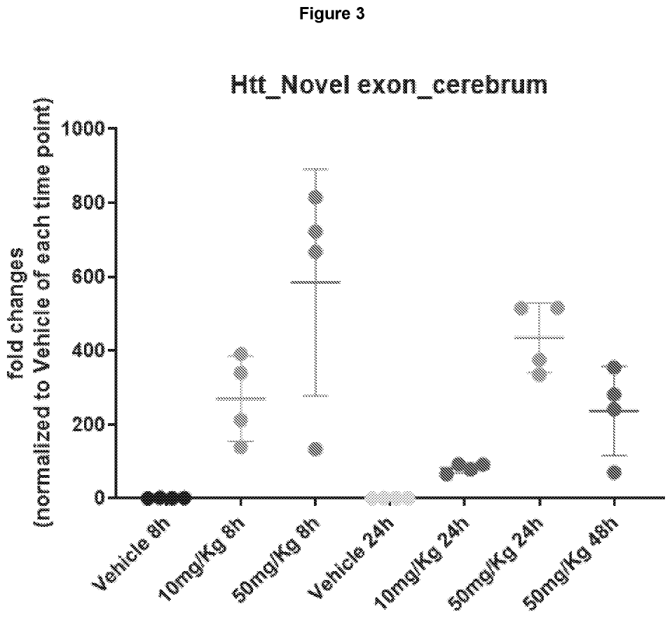

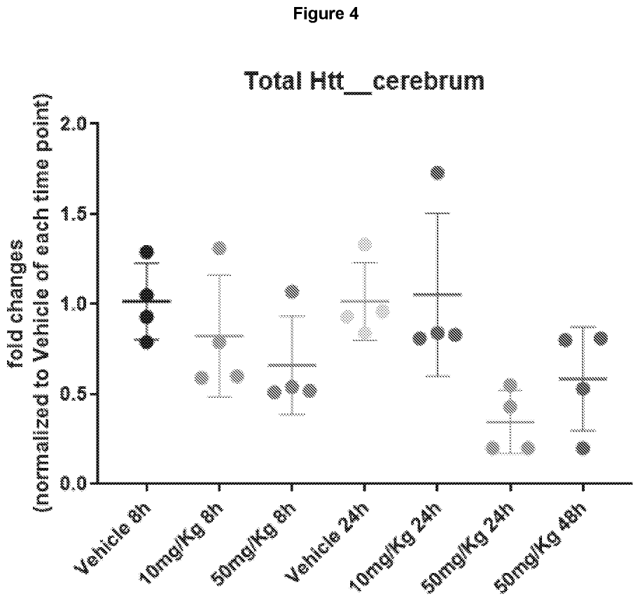

To confirm these findings in vivo a humanized mouse model of HD, the BacHD model (Gray et al, J. Neurosci 2008; 28(24); 6182-6195), which harbors the full length mutant human HTT gene with a CAG expansion of 97 (SEQ ID NO: 23) and expressing mutant protein at ˜1.5× normal mouse HTT was used. BacHD mice received a single oral dose of branaplam at either 10 mg/kg or 50 mg/kg level. Total HTT transcript and novel-exon-containing HTT transcript were measured at 8 hr and 24 h for the 10 mg/kg dose and at 8 h, 24 h and 48 h for the 50 mg/kg dose. Brain tissue (cerebrum, FIGS. 3 and 4) was evaluated by quantitative PCR for changes in levels of total HTT and HTT transcripts containing a novel exon resulting from branaplam treatment. A clear, dose dependent increase in the novel-exon-containing form of HTT was apparent in both brain regions at 8 and 24 h after dosing. Samples from the 50 mg/kg group collected at 48 h after dosing showed a trend towards return to vehicle levels. Total HTT transcript levels at both dose levels showed a lowering trend at 8 h and a greater degree of lowering at the 50 mg/kg level, 24 hours post-dosing.

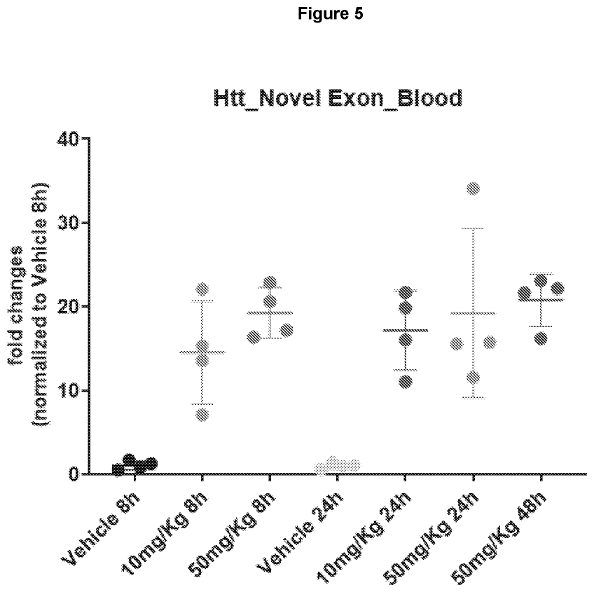

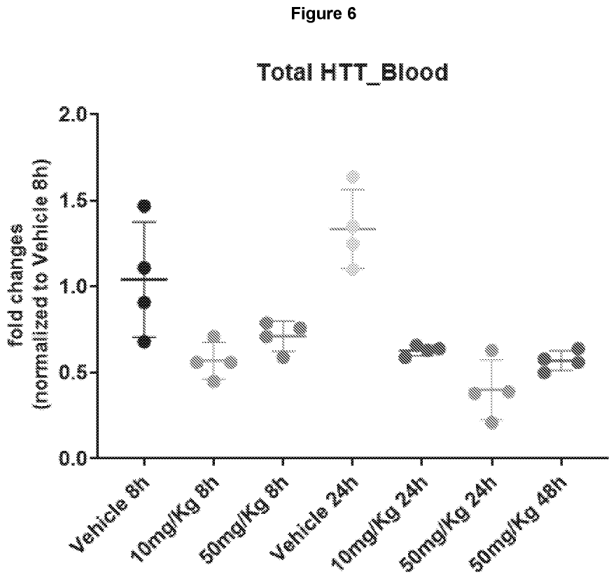

Furthermore, evaluation of blood samples from the same animals revealed robust modulation of novel-exon-containing HTT transcript and lowering of total HTT transcripts (FIGS. 5 and 6).

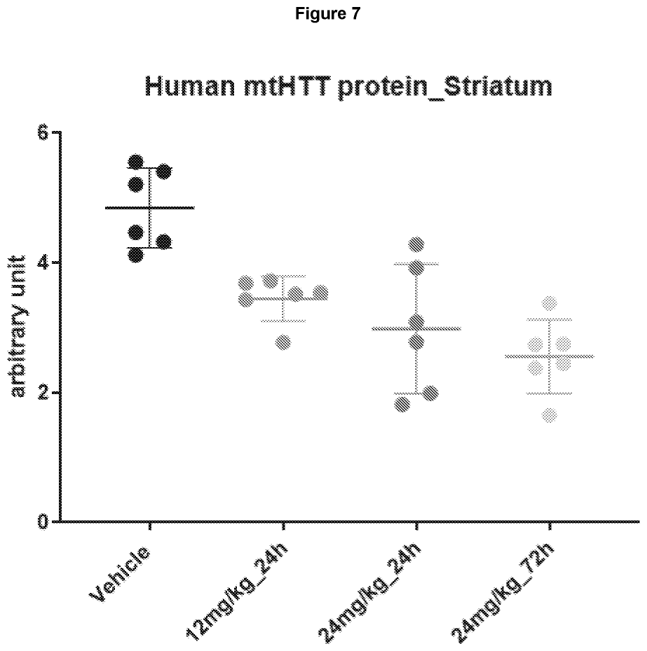

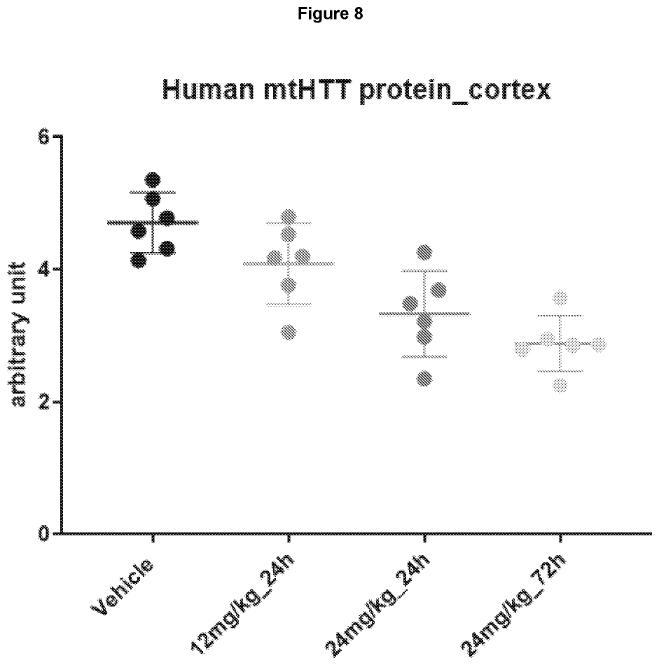

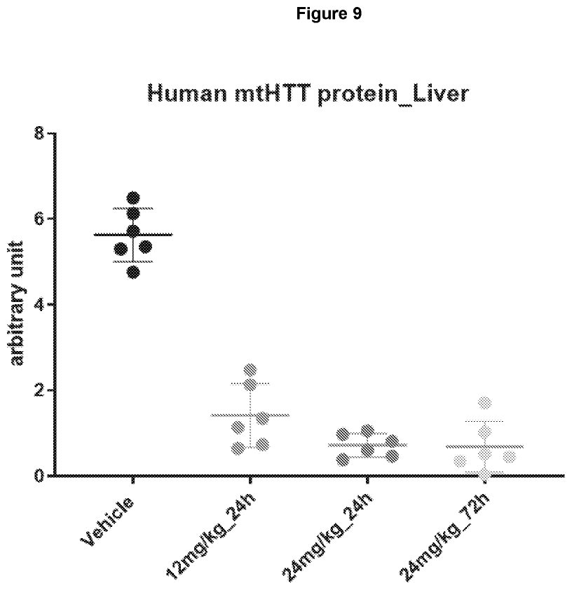

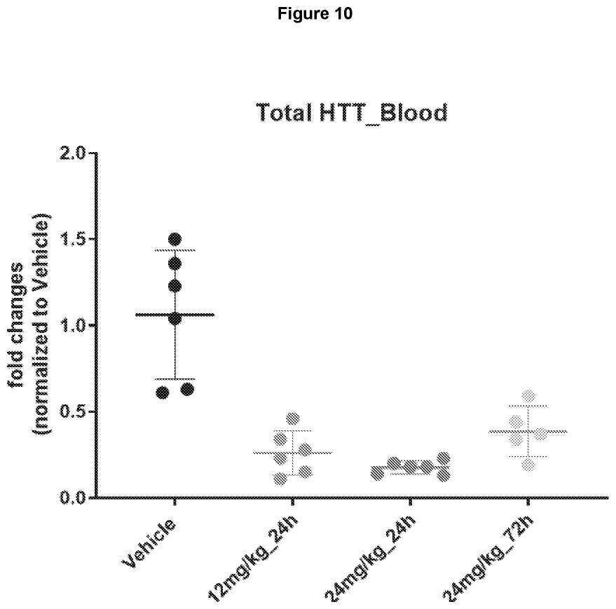

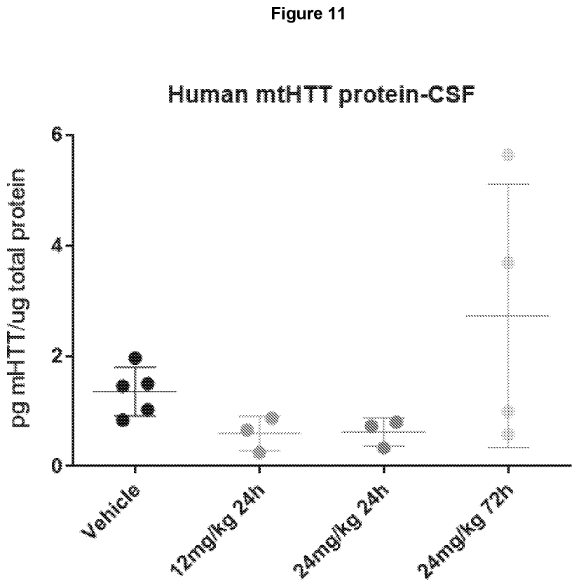

To evaluate the effect of branaplam on mutant HTT protein a repeat dosing study was carried out in the same mouse model. BacHD mice received thrice weekly doses of branaplam at 12 mg/kg or 24 mg/kg for three weeks. Western Blot analysis revealed a dose dependent, significant lowering of mutant HTT protein at the 12 mg/kg dose and at the 24 mg/kg doses 24 h post-dosing in the striatum (FIG. 7) as well as cortex (FIG. 8), with greater HTT reduction evident in the striatum. Evaluation of mutant HTT protein in CSF samples from the same animals revealed a ˜50% lowering at the 12 mg/kg and 24 mg/kg, 24 hrs post-last dose (FIG. 11). A trend towards further lowering of mutant HTT protein, relative to that seen at 24 h was apparent from the 24 mg/kg cohort takedown 72 h post-dose. Additionally, peripheral effect on HTT level was confirmed via robust lowering of mHTT protein in the liver (FIG. 9) and total HTT transcript in blood (FIG. 10). Effects of branaplam are specific to human HTT with no lowering effect seen on endogenous mouse HTT transcript or protein.

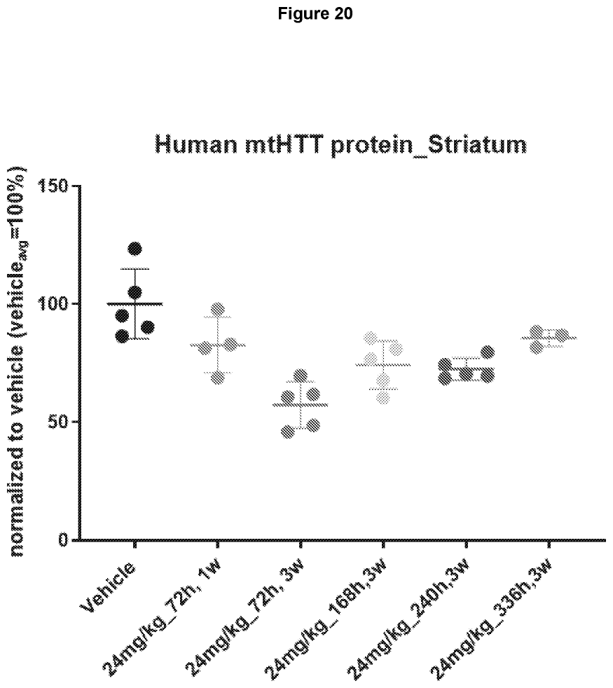

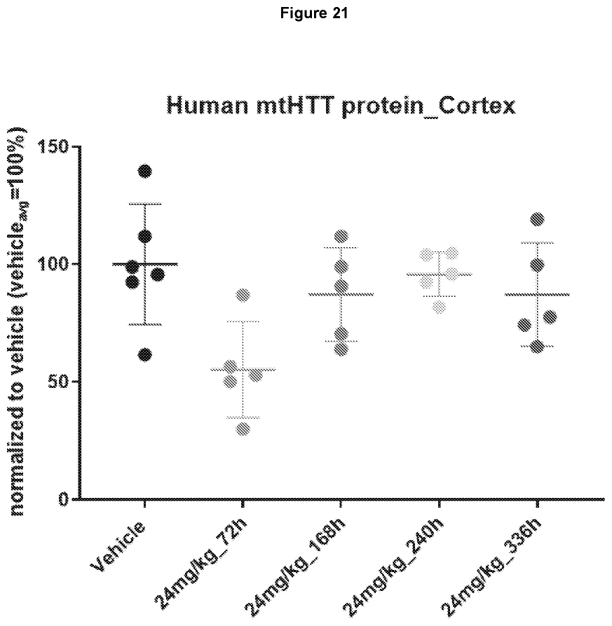

With a view to characterize the time course of HTT protein lowering and recovery and to better model the PK-PD relationship an extended time-course study was performed. BacHD mice received three weekly, oral doses of 24 mg/kg branaplam for three weeks. Animals were taken down and tissues collected 72, 168, 240 or 336 hours after the last dose, An additional cohort of animals received 24 mg/kg dose for one week, with tissues being collected 72 hours after the last dose. Western Blot analysis revealed a time-dependent lowering trend for mutant HTT protein going from 1 week of dosing to 3 weeks of dosing. After 3 weeks of dosing with branaplam maximal HTT protein lowering of approximately 45% (relative to vehicle) was observed at 72 h with a return to baseline between 72-168 h (cortex, FIG. 21) or between 72-336 h (striatum, FIG. 20).

Our results show that intermittent weekly dosing of branaplam results in robust lowering of mutant HTT protein in key areas of the brain (striatum and cortex) impacted in Huntington's disease. The level of mutant HTT protein lowering observed in the CNS is in line with levels expected to provide therapeutic benefit (slowing of HD progression) based on preclinical observations with other HTT lowering modalities (Stanek et al, Hum Gen Ther 2014, 25, 461-474; Kordasiewicz et al, Neuron 2012, 74(6), 1031-1044; Southwell et al, Sci Transl Med 2018, 10, 1-12). Branaplam also lowers peripheral HTT levels offering potential opportunities to address systemic issues (e.g. cardiac, skeletal or metabolic issues) associated with Huntington's disease (van der Burg et al., The Lancet (Neurology) 2009; Vol 8, Issue 8, 765-774).

### Example 1b: Pre-Clinical Evaluation of Further Splicing Modulators: Compounds 1 to 82

### Mutant and Total HTT Multiplex Assay in Q48 HTT Human Embryonic Stem Cell Line

The multiplex assay is performed in human embryonic stem cells (hESCs) which have been derived by Genea Biocells from human blastocysts of HD donors. Cells are plated at a density of 10,000 cells/well into 384-well collagen coated plates and left to adhere for 24 hours, compounds 1 to 82 are then added to cells and incubated for 48 hours (37° C., 5% CO₂), cells are then lysed and the contents split into a black 384-well plate.

The black plates have a combination of HTRF labeled monoclonal antibodies added which recognize discrete areas of the HTT protein, the 2B7-Tb “donor” antibody (0.2 ng/well) recognizes a sequence at the N-terminus of the protein, an MW1-Alexa488 “acceptor 1” antibody (30 ng/well) recognizes an area in the polyQ region, whereas a MAB2166-d2 “acceptor 2” antibody (6 ng/well) recognizes a sequence beyond the polyQ region. The 2B7 antibody was obtained from Coriell (CH02024), the MW1 antibody from Millipore (MABN2427), and the MAB2166 antibody from Millipore (1HU-4C8).

These detection reagents are incubated with the cell lysate at room temperature for 4-6 hours before having their fluorescence quantified at 615 nm (donor) and 535 nm and 665 nm (acceptor 1 and 2 respectively). The donor/acceptor ratio between these signals indicates the relative quantities of mHTT and tHTT proteins. As shown in the Table 1, compounds 1 to 82 as described therein and in Table 2, had the following pIC₅₀ values. For the mHTT pIC₅₀, a pIC₅₀ value below 5 is indicated by zero star ( ), a pIC₅₀ value between 5 and 6 is indicated by a single star (*), a pIC₅₀ value between 6 and 6.5 is indicated by two stars (**), a pIC₅₀ value between 6.5 and 7.0 is indicated by three stars (***), a pIC₅₀ value between 7.0 and 7.5 is indicated by four stars (****) and a pIC₅₀ value above 7.5 is indicated by five stars (*****).

TABLE 1

| | | | Rating | |
|---|---|---|---|---|
| | | | (based on | |
| | | mHTT | mHTT | tHTT |
| Compound | Reference | pIC₅₀ | potency) | pIC₅₀ |
| 1 | WO2014028459 | 7.75 | ***** | 7.8065 |
| | Example 14-1 | | | |
| 2 | WO2015/017589 | 7.73 | ***** | 7.6555 |
| | Example 35-1 | | | |
| 3 | WO2019028440 | 7.717 | ***** | 7.609 |
| | Example 4 | | | |
| 4 | WO2014028459 | 7.6 | ***** | 7.5845 |
| | Example 15-1 | | | |
| 5 | WO2014028459 | 7.591 | ***** | 7.5935 |
| | Example 41-7 | | | |
| 6 | WO2014116845 | 7.585 | ***** | 7.493 |
| | Example 17 | | | |
| 7 | WO2014028459 | 7.5265 | ***** | 7.51 |
| | Example 40-5 | | | |
| 8 | WO2014028459 | 7.4855 | **** | 7.4435 |
| | Example 41-3 | | | |
| 9 | WO2014116845 | 7.4435 | **** | 7.353 |
| | Example 97 | | | |
| 10 | WO2015/017589 | 7.4395 | **** | 7.3875 |
| | Example 30-1 | | | |
| 11 | WO2015/017589 | 7.3875 | **** | 7.3965 |
| | Example 29-3 | | | |
| 12 | WO2014028459 | 7.379 | **** | 7.2825 |
| | Example 30-1 | | | |
| 13 | WO2014116845 | 7.3545 | **** | 7.383 |
| | Example 93 | | | |
| 14 | WO2014028459 | 7.35 | **** | 7.389 |
| | Example 34-1 | | | |
| 15 | WO2015/017589 | 7.349 | **** | 7.283 |
| | Example 6-2 | | | |
| 16 | WO2014116845 | 7.3285 | **** | 7.481 |
| | Example 15 | | | |
| 17 | WO2015/017589 | 7.2895 | **** | 7.424 |
| | Example 15-3 | | | |
| 18 | WO2014028459 | 7.2795 | **** | 7.371 |
| | Example 17-13 | | | |
| 19 | WO2014028459 | 7.2235 | **** | 7.178 |
| | Example 9-1 | | | |
| 20 | WO2014028459 | 7.22 | **** | 7.106 |
| | Example 26-1 | | | |
| 21 | WO2015/017589 | 7.203 | **** | 7.221 |
| | Example 26-5 | | | |
| 22 | WO2014116845 | 7.1945 | **** | 7.1125 |
| | Example 54 | | | |
| 23 | WO2014028459 | 7.1895 | **** | 7.17 |
| | Example 40-3 | | | |
| 24 | WO2015/017589 | 7.186 | **** | 7.168 |
| | Example 35-2 | | | |
| 25 | WO2015017589 | 7.161 | **** | 7.128 |
| | Example 3-6 | | | |
| 26 | WO2014028459 | 7.1215 | **** | 7.0435 |
| | Example 41-14 | | | |
| 27 | WO2014116845 | 7.1125 | **** | 7.085 |
| | Example 96 | | | |
| 28 | WO2015017589 | 7.111 | **** | 7.1045 |
| | Example 34-2 | | | |
| 29 | WO2014028459 | 7.0855 | **** | 7.074 |
| | Example 41-11 | | | |
| 30 | WO2015/017589 | 7.08 | **** | 7.0835 |
| | Example 36-2 | | | |
| 31 | WO2015/017589 | 7.0275 | **** | 7.0015 |
| | Example 3-2 | | | |
| 32 | WO2014028459 | 7.0085 | **** | 7.0625 |
| | Example 43-3 | | | |
| 33 | WO2014028459 | 7.0085 | **** | 7.001 |
| | Example 35-5 | | | |
| 34 | WO2014028459 | 7.004 | **** | 6.9475 |
| | Example 17-12 | | | |
| 35 | WO2014028459 | 7.003 | **** | 6.877 |
| | Example 41-20 | | | |
| 36 | WO2014116845 | 6.996 | *** | 6.827 |
| | Example 100 | | | |
| 37 | WO2014028459 | 6.98 | *** | 7.121 |
| | Example 20-1 | | | |
| 38 | WO2015/017589 | 6.9735 | *** | 7.0015 |
| | Example 18-3 | | | |
| 39 | WO2015/017589 | 6.946 | *** | 6.9105 |
| | Example 13-1 | | | |
| 40 | WO2015/017589 | 6.927 | *** | 6.994 |
| | Example 34-1 | | | |
| 41 | WO2014028459 | 6.923 | *** | 6.8125 |
| | Example 3-1 | | | |
| 42 | WO2015/017589 | 6.92 | *** | 6.885 |
| | Example 18-1 | | | |
| 43 | WO2014116845 | 6.917 | *** | 6.852 |
| | Example 89 | | | |
| 44 | WO2019005980 | 6.869 | *** | 6.7695 |
| | Example 864 | | | |
| 45 | WO2014116845 | 6.861 | *** | 6.7715 |
| | Example 52 | | | |
| 46 | WO2015/017589 | 6.8485 | *** | 6.778 |
| | Example 18-2 | | | |
| 47 | WO2015/017589 | 6.8415 | *** | 6.92 |
| | Example 2-1 | | | |
| 48 | WO2014028459 | 6.822 | *** | 6.9 |
| | Example 1-4 | | | |
| 49 | WO2014116845 | 6.819 | *** | 6.6665 |
| | Example 18 | | | |
| 50 | WO2015/017589 | 6.8055 | *** | 6.7835 |
| | Example 12-1 | | | |
| 51 | WO2015/017589 | 6.801 | *** | 6.779 |
| | Example 26-4 | | | |
| 52 | WO2015/017589 | 6.7625 | *** | 6.6455 |
| | Example 4-1 | | | |
| 53 | WO2015/017589 | 6.746 | *** | 6.7095 |
| | Example 20-3 | | | |
| 54 | WO2014028459 | 6.728 | *** | 6.753 |
| | Example 28-1 | | | |
| 55 | WO2015/017589 | 6.71 | *** | 6.7485 |
| | Example 22-1 | | | |
| 56 | WO2015/017589 | 6.709 | *** | 6.709 |
| | Example 8-1 | | | |
| 57 | WO2014028459 | 6.7085 | *** | 6.791 |
| | Example 24-6 | | | |
| 58 | WO2014028459 | 6.698 | *** | 6.6555 |
| | Example 25-2 | | | |
| 59 | WO2014028459 | 6.678 | *** | 6.5065 |
| | Example 42-9 | | | |
| 60 | WO2015/017589 | 6.6725 | *** | 6.7 |
| | Example 25-1 | | | |
| 61 | WO2014028459 | 6.661 | *** | 6.659 |
| | Example 41-15 | | | |
| 62 | WO2014116845 | 6.6555 | *** | 6.6405 |
| | Example 74 | | | |
| 63 | WO2015/017589 | 6.63 | *** | 6.534 |
| | Example 10-5 | | | |
| 64 | WO2015/017589 | 6.5945 | *** | 6.5885 |
| | Example 26-2 | | | |
| 65 | WO2014116845 | 6.5865 | *** | 6.5835 |
| | Example 63 | | | |
| 66 | WO2014028459 | 6.5815 | *** | 6.637 |
| | Example 35-1 | | | |
| 67 | WO2014028459 | 6.547 | *** | 6.452 |
| | Example 41-4 | | | |
| 68 | WO2014028459 | 6.529 | *** | 6.488 |
| | Example 24-12 | | | |
| 69 | WO2017100726 | 7.1525 | **** | 7.089 |
| | Example 436 | | | |
| 70 | WO2014028459 | 5.999 | * | 5.806 |
| | Example 17-3 or 17-4 | | | |
| 71 | WO2014028459 | 5.996 | * | 5.997 |
| | Example 19-7 | | | |
| 72 | WO2014028459 | 6.21 | ** | 6.13 |
| | Example 31-1 | | | |
| 73 | WO2014028459 | 6.361 | ** | 6.406 |
| | Example 38-1 | | | |
| 74 | WO2014028459 | 6.03 | ** | 6.02 |
| | Example 43-2 | | | |
| 75 | WO2014116845 | 6.4375 | ** | 6.4335 |
| | Example 86 | | | |
| 76 | WO2014116845 | 6.0645 | ** | 6.075 |
| | Example 76 | | | |
| 77 | WO2014116845 | 6.137 | ** | 6.1615 |
| | Example 108 | | | |
| 78 | WO2014116845 | 6.264 | ** | 6.152 |
| | Example 92 | | | |
| 79 | WO2014116845 | 5.9315 | * | 6.01 |
| | Example 46 | | | |
| 80 | WO2014116845 | 6.2465 | ** | 6.071 |
| | Example 61 | | | |
| 81 | WO2018/232039 | <5 | | <5 |
| | Example 16 | | | |
| 82 | WO2014028459A1 | <5 | | <5 |
| | Example 21-1 | | | |

TABLE 2

| Compound | Structure | Name |
|---|---|---|
| 1 | 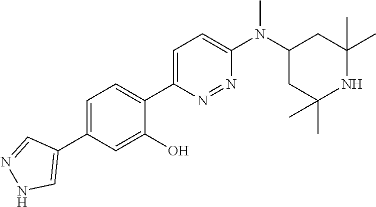 [SMILES: CN(c1ccc(-c2ccc(-c3cn[nH]c3)cc2O)nn1)C1CC(C)(C)NC(C)(C)C1] | 2-(6-(methyl(2,2,6,6- tetramethylpiperidin-4- yl)amino)pyridazin-3-yl)-5- (1H-pyrazol-4-yl)phenol |
| 2 | 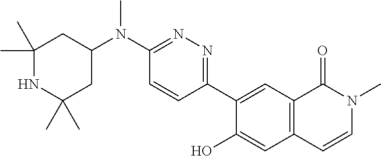 | 6-hydroxy-2-methyl-7-(6- (methyl(2,2,6,6- tetramethylpiperidin-4- yl)amino)pyridazin-3- yl)isoquinolin-1(2H)-one |
| 3 | 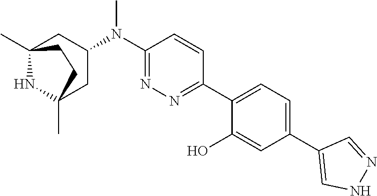 | 2-(6-(((1R,5S)-1,5- dimethyl-8- azabicyclo[3.2.1]octan-3- yl)(methyl)amino)pyridazin- 3-yl)-5-(1H-pyrazol-4- yl)phenol |
| 4 | 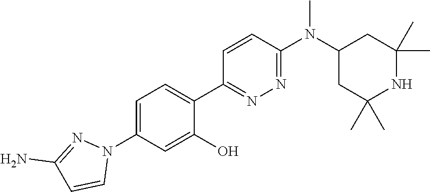 [SMILES: CN(c1ccc(-c2ccc(-n3ccc(N)n3)cc2O)nn1)C1CC(C)(C)NC(C)(C)C1] | 5-(3-amino-1H-pyrazol-1- yl)-2-(6-(methyl(2,2,6,6- tetramethylpiperidin-4- yl)amino)pyridazin-3- yl)phenol |
| 5 | 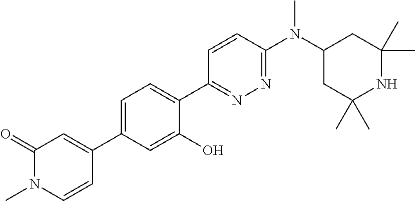 [SMILES: CN(c1ccc(-c2ccc(-c3ccn(C)c(=O)c3)cc2O)nn1)C1CC(C)(C)NC(C)(C)C1] | 4-(3-hydroxy-4-(6- (methyl(2,2,6,6- tetramethylpiperidin-4- yl)amino)pyridazin-3- yl)phenyl)-1-methylpyridin- 2(1H)-one |
| 6 | 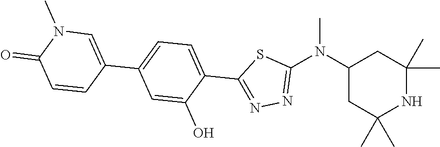 [SMILES: CN(c1nnc(-c2ccc(-c3ccc(=O)n(C)c3)cc2O)s1)C1CC(C)(C)NC(C)(C)C1] | 5-(3-hydroxy-4-(5- (methyl(2,2,6,6- tetramethylpiperidin-4- yl)amino)-1,3,4-thiadiazol- 2-yl)phenyl)-1- methylpyridin-2(1H)-one |
| 7 | 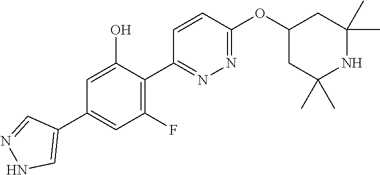 [SMILES: CC1(C)CC(Oc2ccc(-c3c(O)cc(-c4cn[nH]c4)cc3F)nn2)CC(C)(C)N1] | 3-fluoro-5-(1H-pyrazol-4- yl)-2-(6-((2,2,6,6- tetramethylpiperidin-4- yl)oxy)pyridazin-3- yl)phenol |
| 8 | 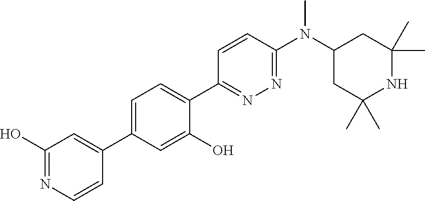 [SMILES: CN(c1ccc(-c2ccc(-c3ccnc(O)c3)cc2O)nn1)C1CC(C)(C)NC(C)(C)C1] | 4-(3-hydroxy-4-(6- (methyl(2,2,6,6- tetramethylpiperidin-4- yl)amino)pyridazin-3- yl)phenyl)pyridin-2-ol |
| 9 | 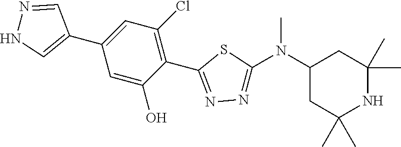 [SMILES: CN(c1nnc(-c2c(O)cc(-c3cn[nH]c3)cc2Cl)s1)C1CC(C)(C)NC(C)(C)C1] | 3-chloro-2-(5- (methyl(2,2,6,6- tetramethylpiperidin-4- yl)amino)-1,3,4-thiadiazol- 2-yl)-5-(1H-pyrazol-4- yl)phenol |
| 10 | 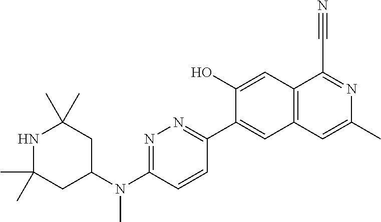 | 7-hydroxy-3-methyl-6-(6- (methyl(2,2,6,6- tetramethylpiperidin-4- yl)amino)pyridazin-3- yl)isoquinoline-1- carbonitrile |
| 11 | 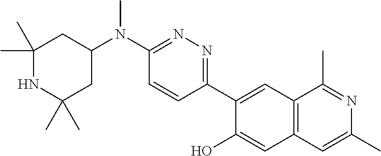 | 1,3-dimethyl-7-(6- (methyl(2,2,6,6- tetramethylpiperidin-4- yl)amino)pyridazin-3- yl)isoquinolin-6-ol |
| 12 | 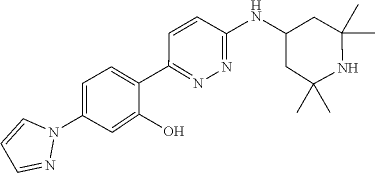 [SMILES: CC1(C)CC(Nc2ccc(-c3ccc(-n4cccn4)cc3O)nn2)CC(C)(C)N1] | 5-(1H-pyrazol-1-yl)-2-(6- ((2,2,6,6- tetramethylpiperidin-4- yl)amino)pyridazin-3- yl)phenol |
| 13 | 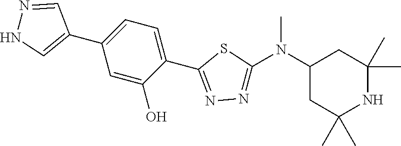 [SMILES: CN(c1nnc(-c2ccc(-c3cn[nH]c3)cc2O)s1)C1CC(C)(C)NC(C)(C)C1] | 2-(5-(methyl(2,2,6,6- tetramethylpiperidin-4- yl)amino)-1,3,4-thiadiazol- 2-yl)-5-(1H-pyrazol-4- yl)phenol |
| 14 | 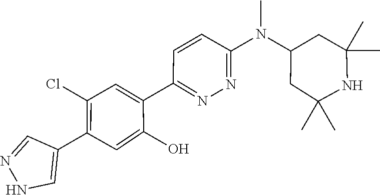 [SMILES: CN(c1ccc(-c2cc(Cl)c(-c3cn[nH]c3)cc2O)nn1)C1CC(C)(C)NC(C)(C)C1] | 4-chloro-2-(6- (methyl(2,2,6,6- tetramethylpiperidin-4- yl)amino)pyridazin-3-yl)-5- (1H-pyrazol-4-yl)phenol |
| 15 | 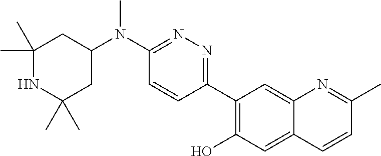 | 2-methyl-7-(6- (methyl(2,2,6,6- tetramethylpiperidin-4- yl)amino)pyridazin-3- yl)quinolin-6-ol |
| 16 | 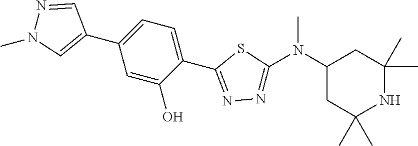 | 2-(5-(methyl(2,2,6,6- tetramethylpiperidin-4- yl)amino)-1,3,4-thiadiazol- 2-yl)-5-(1-methyl-1H- pyrazol-4-yl)phenol |
| 17 | 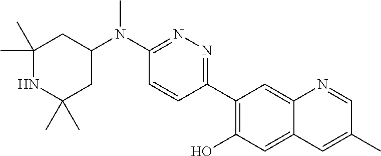 | 3-methyl-7-(6- (methyl(2,2,6,6- tetramethylpiperidin-4- yl)amino)pyridazin-3- yl)quinolin-6-ol |
| 18 | 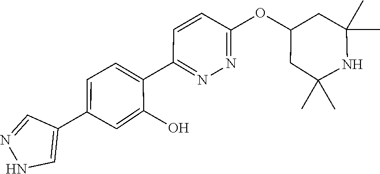 [SMILES: CC1(C)CC(Oc2ccc(-c3ccc(-c4cn[nH]c4)cc3O)nn2)CC(C)(C)N1] | 5-(1H-pyrazol-4-yl)-2-(6- ((2,2,6,6- tetramethylpiperidin-4- yl)oxy)pyridazin-3- yl)phenol |
| 19 | 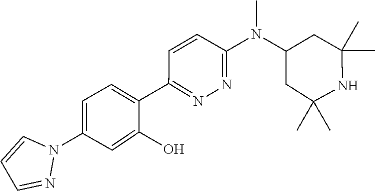 [SMILES: CN(c1ccc(-c2ccc(-n3cccn3)cc2O)nn1)C1CC(C)(C)NC(C)(C)C1] | 2-(6-(methyl(2,2,6,6- tetramethylpiperidin-4- yl)amino)pyridazin-3-yl)-5- (1H-pyrazol-1-yl)phenol |
| 20 | 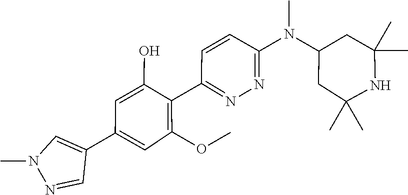 | 3-methoxy-2-(6- (methyl(2,2,6,6- tetramethylpiperidin-4- yl)amino)pyridazin-3-yl)-5- (1-methyl-1H-pyrazol-4- yl)phenol |
| 21 | 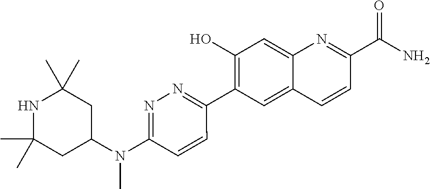 [SMILES: CN(c1ccc(-c2cc3ccc(C(N)=O)nc3cc2O)nn1)C1CC(C)(C)NC(C)(C)C1] | 7-hydroxy-6-(6- (methyl(2,2,6,6- tetramethylpiperidin-4- yl)amino)pyridazin-3- yl)quinoline-2-carboxamide |
| 22 | 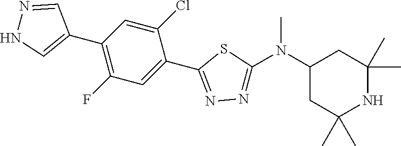 [SMILES: CN(c1nnc(-c2cc(F)c(-c3cn[nH]c3)cc2Cl)s1)C1CC(C)(C)NC(C)(C)C1] | 5-(2-chloro-5-fluoro-4-(1H- pyrazol-4-yl)phenyl)-N- methyl-N-(2,2,6,6- tetramethylpiperidin-4-yl)- 1,3,4-thiadiazol-2-amine |
| 23 | 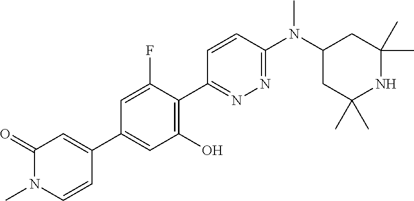 [SMILES: CN(c1ccc(-c2c(O)cc(-c3ccn(C)c(=O)c3)cc2F)nn1)C1CC(C)(C)NC(C)(C)C1] | 4-(3-fluoro-5-hydroxy-4-(6- (methyl(2,2,6,6- tetramethylpiperidin-4- yl)amino)pyridazin-3- yl)phenyl)-1-methylpyridin- 2(1H)-one |
| 24 | 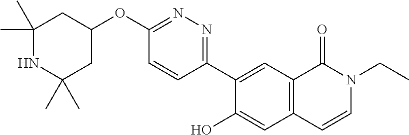 [SMILES: CCn1ccc2cc(O)c(-c3ccc(OC4CC(C)(C)NC(C)(C)C4)nn3)cc2c1=O] | 2-ethyl-6-hydroxy-7-(6- ((2,2,6,6- tetramethylpiperidin-4- yl)oxy)pyridazin-3- yl)isoquinolin-1(2H)-one |
| 25 | 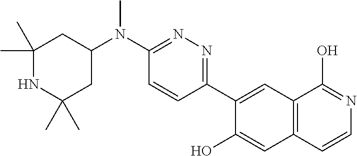 [SMILES: CN(c1ccc(-c2cc3c(O)nccc3cc2O)nn1)C1CC(C)(C)NC(C)(C)C1] | 7-(6-(methyl(2,2,6,6- tetramethylpiperidin-4- yl)amino)pyridazin-3- yl)isoquinoline-1,6-diol |
| 26 | 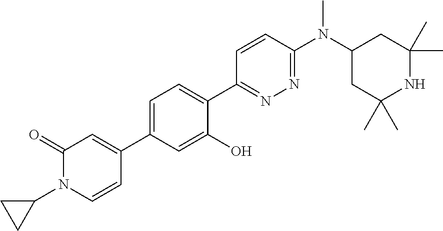 [SMILES: CN(c1ccc(-c2ccc(-c3ccn(C4CC4)c(=O)c3)cc2O)nn1)C1CC(C)(C)NC(C)(C)C1] | 1-cyclopropyl-4-(3- hydroxy-4-(6- (methyl(2,2,6,6- tetramethylpiperidin-4- yl)amino)pyridazin-3- yl)phenyl)pyridin-2(1H)- one |
| 27 | 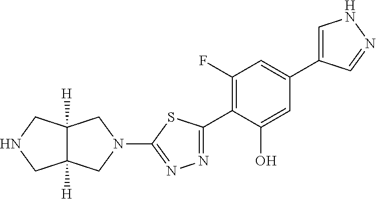 [SMILES: Oc1cc(-c2cn[nH]c2)cc(F)c1-c1nnc(N2C[C@H]3CNC[C@H]3C2)s1] | 3-fluoro-2-(5-((3aR,6aS)- hexahydropyrrolo[3,4- c]pyrrol-2(1H)-yl)-1,3,4- thiadiazol-2-yl)-5-(1H- pyrazol-4-yl)phenol |
| 28 | 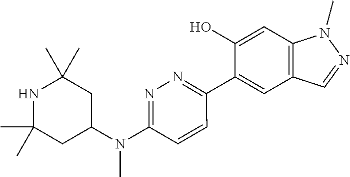 [SMILES: CN(c1ccc(-c2cc3cnn(C)c3cc2O)nn1)C1CC(C)(C)NC(C)(C)C1] | 1-methyl-5-(6- (methyl(2,2,6,6- tetramethylpiperidin-4- yl)amino)pyridazin-3-yl)- 1H-indazol-6-ol |
| 29 | 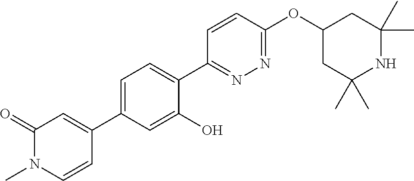 [SMILES: Cn1ccc(-c2ccc(-c3ccc(OC4CC(C)(C)NC(C)(C)C4)nn3)c(O)c2)cc1=O] | 4-(3-hydroxy-4-(6- ((2,2,6,6- tetramethylpiperidin-4- yl)oxy)pyridazin-3- yl)phenyl)-1-methylpyridin- 2(1H)-one |
| 30 | 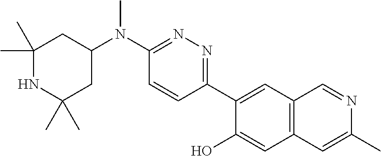 | 3-methyl-7-(6- (methyl(2,2,6,6- tetramethylpiperidin-4- yl)amino)pyridazin-3- yl)isoquinolin-6-ol |
| 31 | 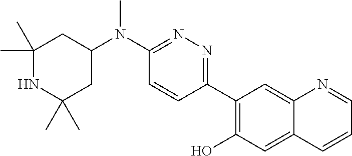 [SMILES: CN(c1ccc(-c2cc3ncccc3cc2O)nn1)C1CC(C)(C)NC(C)(C)C1] | 7-(6-(methyl(2,2,6,6- tetramethylpiperidin-4- yl)amino)pyridazin-3- yl)quinolin-6-ol |
| 32 | 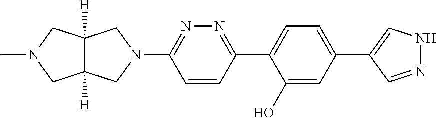 [SMILES: CN1C[C@@H]2CN(c3ccc(-c4ccc(-c5cn[nH]c5)cc4O)nn3)C[C@@H]2C1] | 2-(6-((3aR,6aS)-5- methylhexahydropyrrolo[3, 4-c]pyrrol-2(1H)- yl)pyridazin-3-yl)-5-(1H- pyrazol-4-yl)phenol |
| 33 | 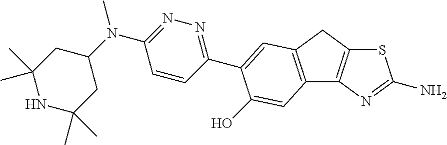 [SMILES: CN(c1ccc(-c2cc3c(cc2O)-c2nc(N)sc2C3)nn1)C1CC(C)(C)NC(C)(C)C1] | 2-amino-6-(6- (methyl(2,2,6,6- tetramethylpiperidin-4- yl)amino)pyridazin-3-yl)- 8H-indeno[1,2-d]thiazol-5- ol |
| 34 | 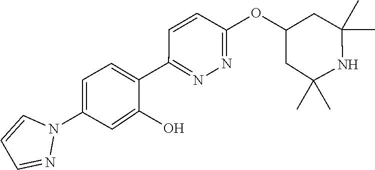 [SMILES: CC1(C)CC(Oc2ccc(-c3ccc(-n4cccn4)cc3O)nn2)CC(C)(C)N1] | 5-(1H-pyrazol-1-yl)-2-(6- (2,2,6,6- tetramethylpiperidin-4- yloxy)pyridazin-3-yl)phenol |
| 35 | 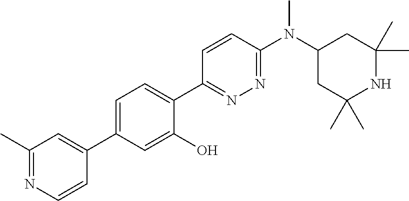 | 2-(6-(methyl(2,2,6,6- tetramethylpiperidin-4- yl)amino)pyridazin-3-yl)-5- (2-methylpyridin-4- yl)phenol |
| 36 | 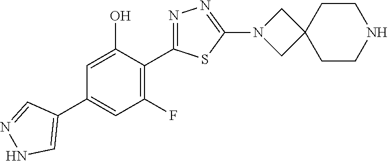 [SMILES: Oc1cc(-c2cn[nH]c2)cc(F)c1-c1nnc(N2CC3(CCNCC3)C2)s1] | 2-(5-(2,7- diazaspiro[3.5]nonan-2-yl)- 1,3,4-thiadiazol-2-yl)-3- fluoro-5-(1H-pyrazol-4- yl)phenol |
| 37 | 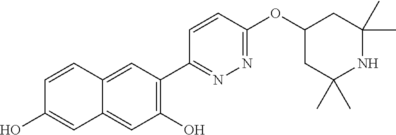 [SMILES: CC1(C)CC(Oc2ccc(-c3cc4ccc(O)cc4cc3O)nn2)CC(C)(C)N1] | 3-(6-(2,2,6,6- tetramethylpiperidin-4- yloxy)pyridazin-3- yl)naphthalene-2,7-diol |
| 38 | 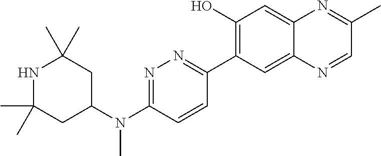 | 3-methyl-7-(6- (methyl(2,2,6,6- tetramethylpiperidin-4- yl)amino)pyridazin-3- yl)quinoxalin-6-ol |
| 39 | 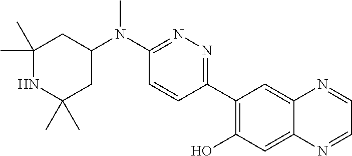 [SMILES: CN(c1ccc(-c2cc3nccnc3cc2O)nn1)C1CC(C)(C)NC(C)(C)C1] | 7-(6-(methyl(2,2,6,6- tetramethylpiperidin-4- yl)amino)pyridazin-3- yl)quinoxalin-6-ol |
| 40 | 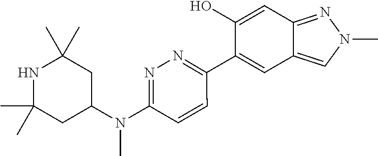 | 2-methyl-5-(6- (methyl(2,2,6,6- tetramethylpiperidin-4- yl)amino)pyridazin-3-yl)- 2H-indazol-6-ol |
| 41 | 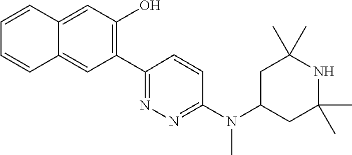 [SMILES: CN(c1ccc(-c2cc3ccccc3cc2O)nn1)C1CC(C)(C)NC(C)(C)C1] | 3-(6-(methyl(2,2,6,6- tetramethylpiperidin-4- yl)amino)pyridazin-3- yl)naphthalen-2-ol |
| 42 | 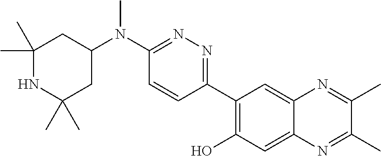 | 2,3-dimethyl-7-(6- (methyl(2,2,6,6- tetramethylpiperidin-4- yl)amino)pyridazin-3- yl)quinoxalin-6-ol |
| 43 | 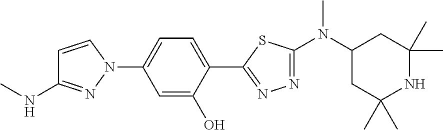 | 2-(5-(methyl(2,2,6,6- tetramethylpiperidin-4- yl)amino)-1,3,4-thiadiazol- 2-yl)-5-(3-(methylamino)- 1H-pyrazol-1-yl)phenol |
| 44 | 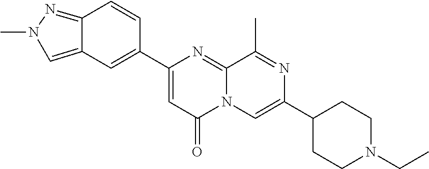 [SMILES: CCN1CCC(c2cn3c(=O)cc(-c4ccc5nn(C)cc5c4)nc3c(C)n2)CC1] | 7-(1-ethylpiperidin-4-yl)-9- methyl-2-(2-methyl-2H- indazol-5-yl)-4H- pyrazino[1,2-a]pyrimidin-4- one |
| 45 | 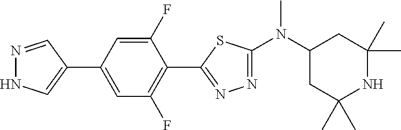 [SMILES: CN(c1nnc(-c2c(F)cc(-c3cn[nH]c3)cc2F)s1)C1CC(C)(C)NC(C)(C)C1] | 5-(2,6-difluoro-4-(1H- pyrazol-4-yl)phenyl)-N- methyl-N-(2,2,6,6- tetramethylpiperidin-4-yl)- 1,3,4-thiadiazol-2-amine |
| 46 | 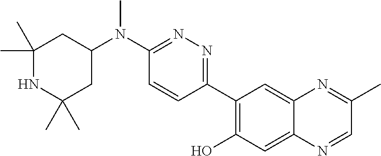 | 2-methyl-7-(6- (methyl(2,2,6,6- tetramethylpiperidin-4- yl)amino)pyridazin-3- yl)quinoxalin-6-ol |
| 47 | 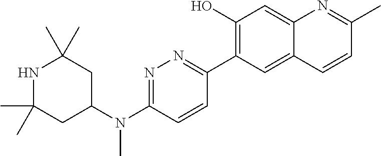 | 2-methyl-6-(6- (methyl(2,2,6,6- tetramethylpiperidin-4- yl)amino)pyridazin-3- yl)quinolin-7-ol |
| 48 | 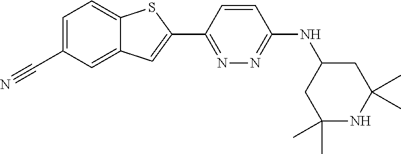 [SMILES: CC1(C)CC(Nc2ccc(-c3cc4cc(C#N)ccc4s3)nn2)CC(C)(C)N1] | 2-(6-(2,2,6,6- tetramethylpiperidin-4- ylamino)pyridazin-3- yl)benzo[b]thiophene-5- carbonitrile |
| 49 | 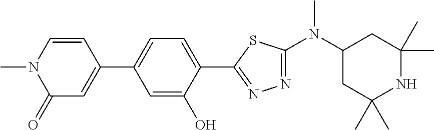 | 4-(3-hydroxy-4-(5- (methyl(2,2,6,6- tetramethylpiperidin-4- yl)amino)-1,3,4-thiadiazol- 2-yl)phenyl)-1- methylpyridin-2(1H)-one |
| 50 | 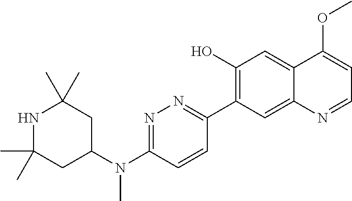 | 4-methoxy-7-(6- (methyl(2,2,6,6- tetramethylpiperidin-4- yl)amino)pyridazin-3- yl)quinolin-6-ol |
| 51 | 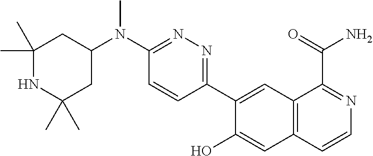 [SMILES: CN(c1ccc(-c2cc3c(C(N)=O)nccc3cc2O)nn1)C1CC(C)(C)NC(C)(C)C1] | 6-hydroxy-7-(6- (methyl(2,2,6,6- tetramethylpiperidin-4- yl)amino)pyridazin-3- yl)isoquinoline-1- carboxamide |
| 52 | 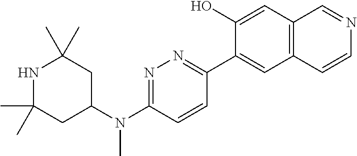 [SMILES: CN(c1ccc(-c2cc3ccncc3cc2O)nn1)C1CC(C)(C)NC(C)(C)C1] | 6-(6-(methyl(2,2,6,6- tetramethylpiperidin-4- yl)amino)pyridazin-3- yl)isoquinolin-7-ol |
| 53 | 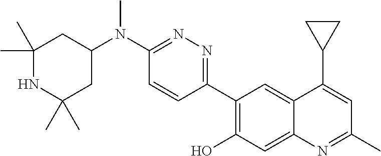 | 4-cyclopropyl-2-methyl-6- (6-(methyl(2,2,6,6- tetramethylpiperidin-4- yl)amino)pyridazin-3- yl)quinolin-7-ol |
| 54 | 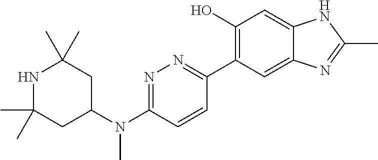 | 2-methyl-5-(6- (methyl(2,2,6,6- tetramethylpiperidin-4- yl)amino)pyridazin-3-yl)- 1H-benzo[d]imidazol-6-ol |
| 55 | 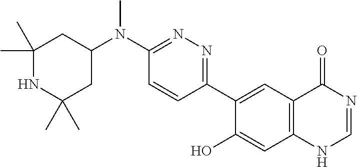 [SMILES: CN(c1ccc(-c2cc3c(=O)nc[nH]c3cc2O)nn1)C1CC(C)(C)NC(C)(C)C1] | 7-hydroxy-6-(6- (methyl(2,2,6,6- tetramethylpiperidin-4- yl)amino)pyridazin-3- yl)quinazolin-4(1H)-one |
| 56 | 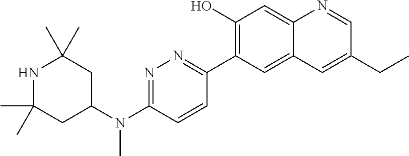 [SMILES: CCc1cnc2cc(O)c(-c3ccc(N(C)C4CC(C)(C)NC(C)(C)C4)nn3)cc2c1] | 3-ethyl-6-(6- (methyl(2,2,6,6- tetramethylpiperidin-4- yl)amino)pyridazin-3- yl)quinolin-7-ol |
| 57 | 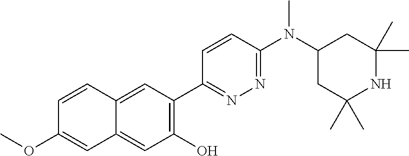 | 7-methoxy-3-(6- (methyl(2,2,6,6- tetramethylpiperidin-4- yl)amino)pyridazin-3- yl)naphthalen-2-ol |
| 58 | 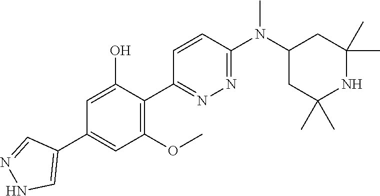 [SMILES: COc1cc(-c2cn[nH]c2)cc(O)c1-c1ccc(N(C)C2CC(C)(C)NC(C)(C)C2)nn1] | 3-methoxy-2-(6- (methyl(2,2,6,6- tetramethylpiperidin-4- yl)amino)pyridazin-3-yl)-5- (1H-pyrazol-4-yl)phenol |
| 59 | 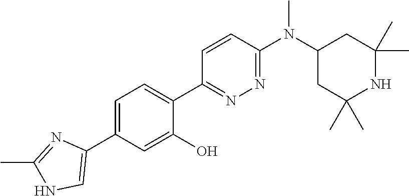 | 2-(6-(methyl(2,2,6,6- tetramethylpiperidin-4- yl)amino)pyridazin-3-yl)-5- (2-methyl-1H-imidazol-4- yl)phenol |
| 60 | 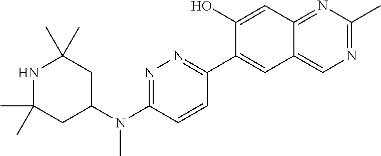 | 2-methyl-6-(6- (methyl(2,2,6,6- tetramethylpiperidin-4- yl)amino)pyridazin-3- yl)quinazolin-7-ol |
| 61 | 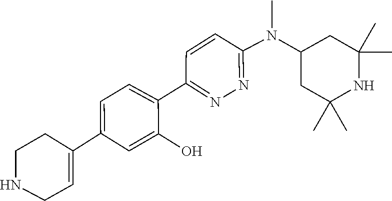 [SMILES: CN(c1ccc(-c2ccc(C3=CCNCC3)cc2O)nn1)C1CC(C)(C)NC(C)(C)C1] | 2-(6-(methyl(2,2,6,6- tetramethylpiperidin-4- yl)amino)pyridazin-3-yl)-5- (1,2,3,6-tetrahydropyridin- 4-yl)phenol |
| 62 | 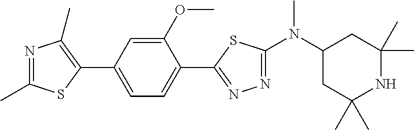 | 5-(4-(2,4-dimethylthiazol-5- yl)-2-methoxyphenyl)-N- methyl-N-(2,2,6,6- tetramethylpiperidin-4-yl)- 1,3,4-thiadiazol-2-amine |
| 63 | 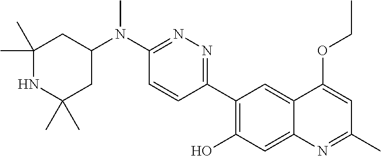 | 4-ethoxy-2-methyl-6-(6- (methyl(2,2,6,6- tetramethylpiperidin-4- yl)amino)pyridazin-3- yl)quinolin-7-ol |
| 64 | 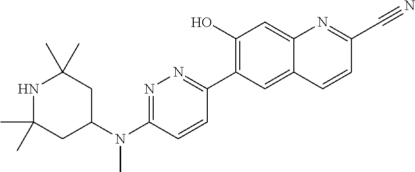 [SMILES: CN(c1ccc(-c2cc3ccc(C#N)nc3cc2O)nn1)C1CC(C)(C)NC(C)(C)C1] | 7-hydroxy-6-(6- (methyl(2,2,6,6- tetramethylpiperidin-4- yl)amino)pyridazin-3- yl)quinoline-2-carbonitrile |
| 65 | 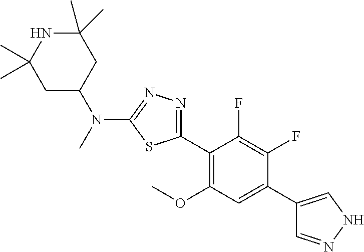 [SMILES: COc1cc(-c2cn[nH]c2)c(F)c(F)c1-c1nnc(N(C)C2CC(C)(C)NC(C)(C)C2)s1] | 5-(2,3-difluoro-6-methoxy- 4-(1H-pyrazol-4-yl)phenyl)- N-methyl-N-(2,2,6,6- tetramethylpiperidin-4-yl)- 1,3,4-thiadiazol-2-amine |
| 66 | 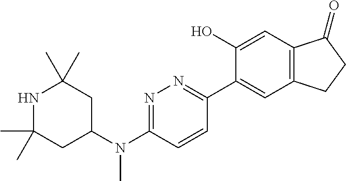 [SMILES: CN(c1ccc(-c2cc3c(cc2O)C(=O)CC3)nn1)C1CC(C)(C)NC(C)(C)C1] | 6-hydroxy-5-(6- (methyl(2,2,6,6- tetramethylpiperidin-4- yl)amino)pyridazin-3-yl)- 2,3-dihydro-1H-inden-1- one |
| 67 | 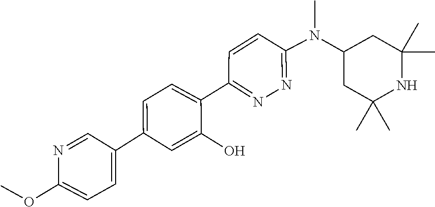 | 5-(6-methoxypyridin-3-yl)- 2-(6-(methyl(2,2,6,6- tetramethylpiperidin-4- yl)amino)pyridazin-3- yl)phenol |
| 68 | 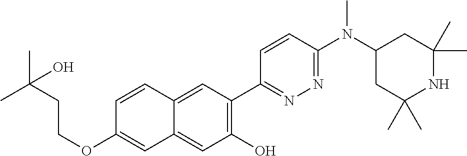 [SMILES: CN(c1ccc(-c2cc3ccc(OCCC(C)(C)O)cc3cc2O)nn1)C1CC(C)(C)NC(C)(C)C1] | 7-(3-hydroxy-3- methylbutoxy)-3-(6- (methyl(2,2,6,6- tetramethylpiperidin-4- yl)amino)pyridazin-3- yl)naphthalen-2-ol |
| 69 | 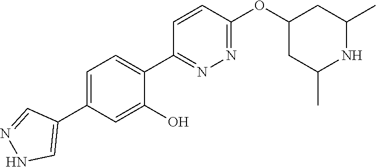 [SMILES: CC1CC(Oc2ccc(-c3ccc(-c4cn[nH]c4)cc3O)nn2)CC(C)N1] | 2-(6-((2,6- dimethylpiperidin-4- yl)oxy)pyridazin-3-yl)-5- (1H-pyrazol-4-yl)phenol |
| 70 | 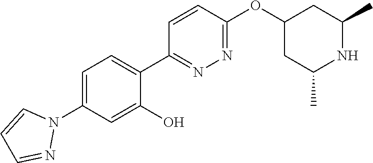 [SMILES: C[C@@H]1CC(Oc2ccc(-c3ccc(-n4cccn4)cc3O)nn2)C[C@@H](C)N1] | 2-(6-(((2R,6R)-2,6- dimethylpiperidin-4- yl)oxy)pyridazin-3-yl)-5- (1H-pyrazol-1-yl)phenol |
| 71 | 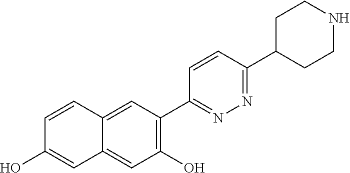 [SMILES: Oc1ccc2cc(-c3ccc(C4CCNCC4)nn3)c(O)cc2c1] | 3-(6-(piperidin-4- yl)pyridazin-3- yl)naphthalene-2,7-diol |
| 72 | 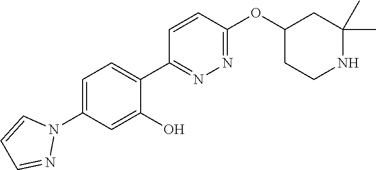 [SMILES: CC1(C)CC(Oc2ccc(-c3ccc(-n4cccn4)cc3O)nn2)CCN1] | 2-(6-((2,2- dimethylpiperidin-4- yl)oxy)pyridazin-3-yl)-5- (1H-pyrazol-1-yl)phenol |
| 73 | 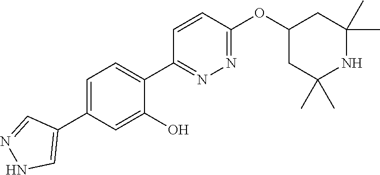 [SMILES: CC1(C)CC(Oc2ccc(-c3ccc(-c4cn[nH]c4)cc3O)nn2)CC(C)(C)N1] | 5-(1H-pyrazol-4-yl)-2-(6- ((2,2,6,6- tetramethylpiperidin-4- yl)methyl)pyridazin-3- yl)phenol |
| 74 | 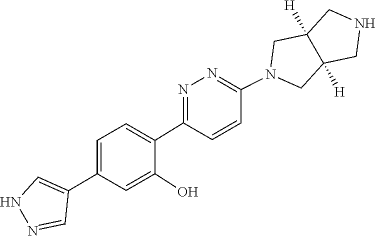 [SMILES: Cc1n[nH]cc1-c1ccc(-c2ccc(N3C[C@H]4CNC[C@H]4C3)nn2)c(O)c1] | 2-(6-((3aR,6aS)- hexahydropyrrolo[3,4- c]pyrrol-2(1H)-yl)pyridazin- 3-yl)-5-(1H-pyrazol-4- yl)phenol |
| 75 | 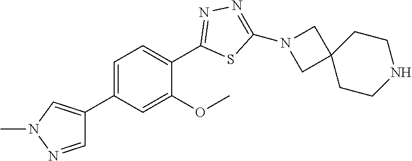 [SMILES: COc1cc(-c2cnn(C)c2)ccc1-c1nnc(N2CC3(CCNCC3)C2)s1] | 2-(2-methoxy-4-(1-methyl- 1H-pyrazol-4-yl)phenyl)-5- (2,7-diazaspiro[3.5]nonan- 2-yl)-1,3,4-thiadiazole |
| 76 | 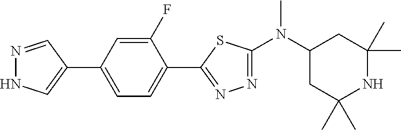 [SMILES: CN(c1nnc(-c2ccc(-c3cn[nH]c3)cc2F)s1)C1CC(C)(C)NC(C)(C)C1] | 5-(2-fluoro-4-(1H-pyrazol- 4-yl)phenyl)-N-methyl-N- (2,2,6,6- tetramethylpiperidin-4-yl)- 1,3,4-thiadiazol-2-amine |
| 77 | 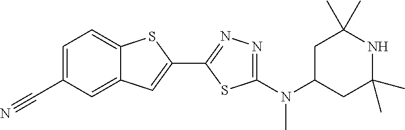 [SMILES: CN(c1nnc(-c2cc3cc(C#N)ccc3s2)s1)C1CC(C)(C)NC(C)(C)C1] | 2-(5-(methyl(2,2,6,6- tetramethylpiperidin-4- yl)amino)-1,3,4-thiadiazol- 2-yl)benzo[b]thiophene-5- carbonitrile |
| 78 | 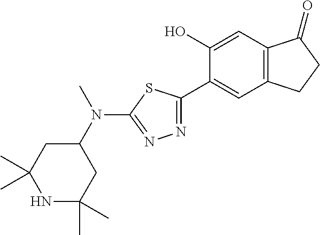 [SMILES: CN(c1nnc(-c2cc3c(cc2O)C(=O)CC3)s1)C1CC(C)(C)NC(C)(C)C1] | 6-hydroxy-5-(5- (methyl(2,2,6,6- tetramethylpiperidin-4- yl)amino)-1,3,4-thiadiazol- 2-yl)-2,3-dihydro-1H-inden- 1-one |
| 79 | 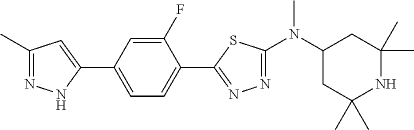 | 5-(2-fluoro-4-(3-methyl-1H- pyrazol-5-yl)phenyl)-N- methyl-N-(2,2,6,6- tetramethylpiperidin-4-yl)- 1,3,4-thiadiazol-2-amine |
| 80 | 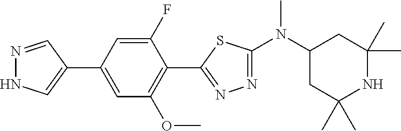 [SMILES: COc1cc(-c2cn[nH]c2)cc(F)c1-c1nnc(N(C)C2CC(C)(C)NC(C)(C)C2)s1] | 5-(2-fluoro-6-methoxy-4- (1H-pyrazol-4-yl)phenyl)- N-methyl-N-(2,2,6,6- tetramethylpiperidin-4-yl)- 1,3,4-thiadiazol-2-amine |
| 81 | 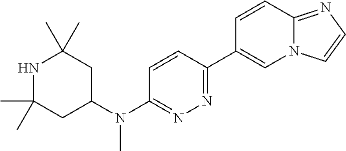 [SMILES: CN(c1ccc(-c2ccc3nccn3c2)nn1)C1CC(C)(C)NC(C)(C)C1] | 6-(imidazo[1,2-a]pyridin-6- yl)-N-methyl-N-(2,2,6,6- tetramethylpiperidin-4- yl)pyridazin-3-amine |
| 82 | 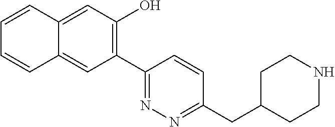 [SMILES: Oc1cc2ccccc2cc1-c1ccc(CC2CCNCC2)nn1] | 3-(6-(piperidin-4- ylmethyl)pyridazin-3- yl)naphthalen-2-ol |

### Example 1c: Dose Selection for Clinical Evaluation of Branaplam

### Example 1c.1: Pharmacokinetic Model Description

The pharmacokinetic (PK) model in adult subjects was developed using an Advanced Compartmental And Transit (ACAT)/Physiologically Based Pharmacokinetic (PBPK) model in GastroPlus™ software, Version 9.6 (SimulationPlus, Lancaster, CA, USA).

### Modeling Strategy

Principle set-up of a pediatric ACAT/PBPK model in GastroPlus™ software including physico-chemical parameters, physiological parameters, and formulation factors. The ACAT model was linked to a full PBPK model in which tissue-to-plasma concentration ratios were estimated via in silico method provided by GastroPlus™ software.
Estimation of the clearance (CL) using plasma concentrations of branaplam from an open-label multi-part first-in-human proof of concept study of oral branaplam, Part 1 only. The aim of Part 1 of this study was to determine the safety and tolerability of ascending weekly doses and to estimate the maximum tolerated dose (MTD) of oral/enteral branaplam (formulation described in Example 3) in infants with Type 1 SMA. All patients had exactly 2 copies of the SMN2 gene. Patients were dosed once weekly with branaplam. The branaplam doses were escalated in subsequent cohorts until MTD was determined or when PK results confirmed that the MTD could not be reached due to a potential pharmacokinetic exposure plateau at higher doses. The starting dose referring to the free base was 6 mg/m² (equal to 0.3125 mg/kg). Subsequent doses were 12 mg/m², 24 mg/m², 48 mg/m² and 60 mg/m² (0.625 mg/kg, 1.25 mg/kg, 2.5 mg/kg and 3.125 mg/kg, respectively). 14 patients were enrolled in Part 1; 13 patients were exposed to branaplam. The duration of exposure ranged from 4-33 months, 7 patients remain in the study. Six of the 7 patients are receiving 60 mg/m², 1 patient is receiving 48 mg/m². No dose-limiting toxicity was observed.
Estimation of in vivo solubility and the in vivo dissolution of branaplam using the plasma concentration-time courses in 33-39 months old Type 1 SMA patients after nominal branaplam doses of 60 mg/m² (3.125 mg/kg, first-in-human proof of concept study with branaplam as described above) and optimization functionality in GastroPlus™ software.
Estimation of distribution parameters of branaplam using concentration-time courses in 33-39 months old Type 1 SMA patients after nominal branaplam doses of 60 mg/m² (3.125 mg/kg, first-in-human proof of concept study with branaplam as described above) and optimization functionality in GastroPlus™ software.
·Qualification of pediatric ACAT/PBPK model by comparison of simulated plasma concentration-time courses with observed courses in 35-44 months old and 22-29 months old Type 1 SMA patients from the first-in-human proof of concept study with branaplam as described above.

### Model Development

Observed plasma concentrations of branaplam in 33-39 months old Type 1 SMA patients after nominal branaplam doses of 60 mg/m² (3.125 mg/kg, first-in-human proof of concept study with branaplam as described above) were used to build the pediatric ACAT/PBPK by means of GastroPlus™ software. In general, drug metabolizing enzyme(s)/transporter(s) are mature at about age of 2 years (Lin W, Yan J H, Heimbach T, et al (2018) Pediatric Physiologically Based Pharmacokinetic Model Development: Current Status and Challenges. Curr Pharmacol Rep; 4: 491-501). CL prediction in children over 2 year old patients can be reliably predicted based on body size, hepatic blood flow rate and other developmental physiological factors (Lin W et al, 2018, see above). The assumption was made, that PK parameters can be transferred to adult population.

The in vivo solubility parameter for branaplam was newly assessed to consider the impact of the solubilizer cyclodextrin (CD), which is present in the currently used formulation (Example 3), on the solubility of branaplam. The Optimization function in GastroPlus™ software was applied to match the observed systemic exposure in 33-39 months old Type 1 SMA patients (nominal dose: 60 mg/m², 3.125 mg/kg, first-in-human proof of concept study with branaplam as described above) and to estimate the in vivo solubility of branaplam in the presence of CD.

In Type 1 SMA patients after oral branaplam administration, the median time of maximum concentration (Tmax) of branaplam in plasma ranged between 2.97 to 4.00 h at all dose levels investigated (nominal dose range: 6 to 60 mg/m², first-in-human proof of concept study with branaplam as described above). Despite the use of the CD-containing branaplam solution suggesting a rapid absorption, the Tmax values indicated a delayed absorption. The Optimization function in GastroPlus™ software was applied to match the observed branaplam plasma concentration-time profile in Type 1 SMA patients with an age of 33-39 months (nominal dose: 60 mg/m², 3.125 mg/kg, first-in-human proof of concept study with branaplam as described above) with the controlled-release dissolution profile resulting in release properties of 2%, 5%, and 95% at 0.25, 1, 3.25 h, respectively.

The pediatric ACAT model was linked to a compartment PK model in plasma (GastroPlus™ software) to describe the plasma concentration-time course of branaplam in Type 1 SMA patients with an age of 33-39 months more accurately (nominal dose range: 6 to 60 mg/m², first-in-human proof of concept study with branaplam as described above). This was executed by means of the Optimization functionality in GastroPlus™ software resulting in fitted distribution parameters (k12, first-order rate constant from compartment 1 to compartment 2; k21, first-order rate constant from compartment 2 to compartment 1; Vc, volume of central compartment).

### Results—Pediatric ACAT/PBPK Model

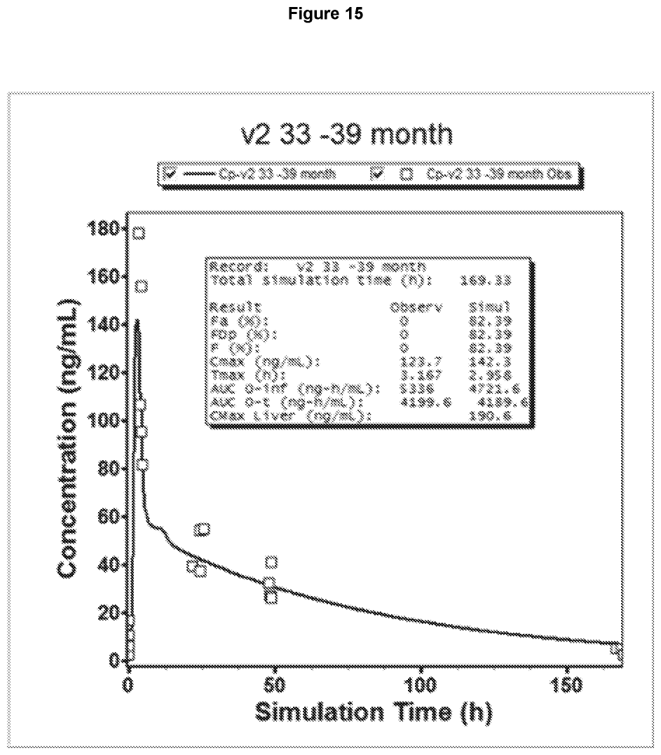

A pediatric ACAT/PBPK model was successfully developed. The model was based on observed plasma concentrations of branaplam in 33-39 months old Type 1 SMA patients after nominal branaplam dose of 60 mg/m² (3.125 mg/kg, first-in-human proof of concept study with branaplam as described above). Used and derived input parameters of the model are presented in Table 3. The simulated plasma concentration-time course of branaplam in 33-39 months old Type 1 SMA patients are presented in FIG. 15.

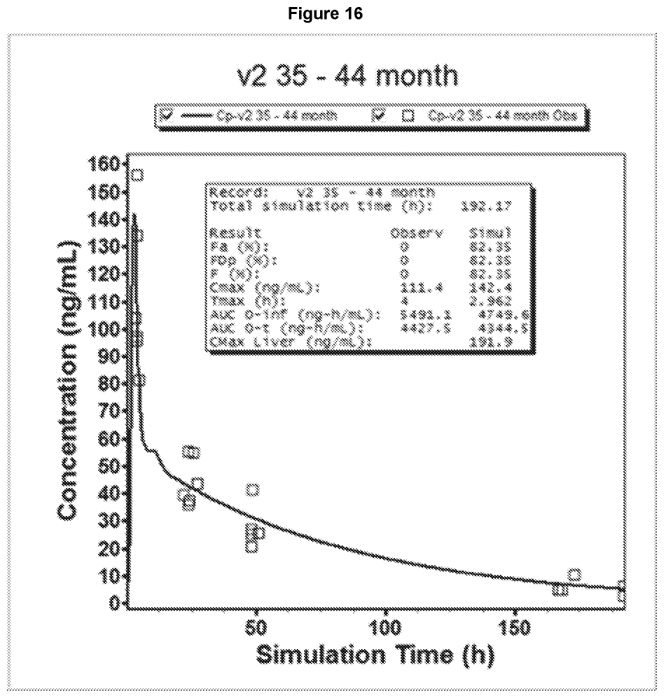

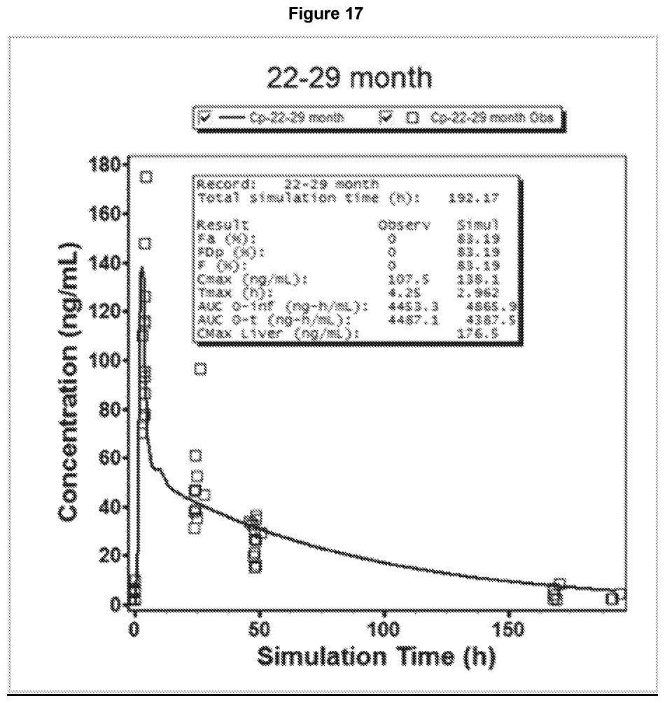

After the pediatric ACAT/PBPK model was constructed, it was further qualified using the branaplam plasma concentration-time courses derived from 35-44 months and 22-29 months old Type 1 SMA patients (nominal dose: 60 mg/m², 3.125 mg/kg, first-in-human proof of concept study with branaplam as described above). The simulated plasma concentration-time course for both patient populations are presented in FIGS. 16 and 17. Both figures showed that the plasma concentration-time course of branaplam simulated by the pediatric ACAT/PBPK model matched appropriately the plasma concentration-time course of additional Type 1 SMA patient populations suggesting an appropriate ACAT/PBPK model development.

TABLE 3
Input parameters for the established branaplam pediatric ACAT/PBPK
model for Type 1 SMA patients, 33-39 months of age

Parameters

| | |
|---|---|
| Dosage form | controlled-released (as described in GastroPlus ™ software; |
| | release profile: 2%, 5%, and 95% at 0.25, 1, 3.25 h, |
| | respectively) |
| LogP | 6.7 (predicted by ADMET predictor module in GastroPlus ™ |
| | software) |
| Solubility (mg/mL) | 0.088 at pH 6.8 (Optimized value using GastroPlus ™ software |
| | based on the observed concentration-time data) |
| pKa | 0.46, 1.47, 8.7 (fitted by GastroPlus ™ on in vitro solubility |
| | information) |
| Dose volume (mL) | 15 |
| Particle density (g/mL) | 1.2 (predicted by ADMET predictor, GastroPlus ™) |
| Mean particle radius (um) | 10 +/− 0, with one particle radius bin |
| Precipitation time (sec) | 100 (predicted by ADMET predictor, GastroPlus ™) |
| Diffusion coefficient (cm²/s * | 0.63 (predicted by ADMET predictor, GastroPlus ™) |
| 10⁵) | |
| Permeability (cm/s * 10⁴) | 2.5 (estimated from Caco-2 permeability ^{a} to human Peff) |
| Simulation time (h) | 168 |
| k12 (1/h) | 1.41 (Optimized value using GastroPlus ™ based on the |
| | observed concentration-time data) |
| k21 (1/h) | 0.144 (Optimized value using GastroPlus ™ based on the |
| | observed concentration-time data) |
| Vc (1/h) | 3.62 (Optimized value using GastroPlus ™ based on the |
| | observed concentration-time data) |
| Patient population parameter: | |
| 33-39 months | body weight: 12.6 kg, absolute dose: 39.2 mg, CL: 6.84 L/kg |
| 35-44 months | body weight: 12.9 kg, absolute dose: 40.2 mg, CL: 6.97 L/kg |
| 22-29 months | body weight: 10.5 kg, absolute dose: 31.1 mg, CL: 5.32 L/kg |

CL: clearance; k12: first-order rate constant between compartment 1 and compartment 2; k21: first-order rate constant between compartment 2 and compartment 1; LogP: logarithm of partition coefficient between organic and aqueous solution; Peff: effective permeability; pKa: negative logarithm of the acid dissociation constant; Vc: volume of central compartment;
^{a} Artursson P, Karlsson J (1991) Correlation between oral drug absorption in humans and apparent drug permeability coefficients in human intestinal epithelial (Caco-2) cells. Biochem Biophys Res Comm; 175: 880-5.

### Scaling-Up Pediatric ACAT/PBPK Model to Adult ACAT/PBPK Model

To scale up the branaplam pediatric ACAT/PBPK model to an adult ACAT/PBPK model, adult CL projected from nonclinical species was applied to replace the pediatric CL, and the body weight of 70 kg was used. Dose volume was set 250 mL. The distribution parameters estimated for the pediatric patients were entered to the adult PK model. Estimated apparent terminal elimination half life (T½) was 62 h in adults). The general input parameters in the adult ACAT/PBPK model are summarized in Table 4. Model predictions of branaplam exposures after single oral administration of branaplam to adults are depicted in Table 5.

TABLE 4
Input parameters for the established branaplam adult ACAT/PBPK model

Parameters

| Dosage form | controlled-released (GastroPlus ™, release profile: 2%, 5%, |
|---|---|
| | and 95% at 0.25, 1, 3.25 h, respectively) |
| LogP | 6.7 (predicted by ADMET predictor, GastroPlus ™) |
| Solubility (mg/mL) | 0.088 at pH 6.8 (Optimized value using GastroPlus ™ based |
| | on the observed concentration-time data) |
| pKa | 0.46, 1.47, 8.7 (fitted by GastroPlus ™ on in vitro solubility |
| | information) |
| Dose volume (mL) | 250 |
| Particle density (g/mL) | 1.2 (predicted by ADMET predictor, GastroPlus ™) |
| Mean particle radius (um) | 10 +/− 0, with one particle radius bin |
| Precipitation time (sec) | 100 (predicted by ADMET predictor, GastroPlus ™) |
| Diffusion coefficient (cm²/s * | 0.63 (predicted by ADMET predictor, GastroPlus ™) |
| 10⁵) | |
| Permeability (cm/s * 10⁴) | 2.5 (estimated from Caco-2 permeability ^{a} to human Peff) |
| Simulation time (h) | 168 |
| CL (L/h/kg) | 0.474 |
| k12 (1/h) | 1.41 (Optimized value using GastroPlus ™ based on the |
| | observed concentration-time data) |
| k21 (1/h) | 0.144 (Optimized value using GastroPlus ™ based on the |
| | observed concentration-time data) |
| Vc (1/h) | 3.62 (Optimized value using GastroPlus ™ based on the |
| | observed concentration-time data) |
| T½ (h) | 62 |
| Body weight (kg) | 70 |

CL: clearance; k12: first-order rate constant between compartment 1 and compartment 2; k21: first-order rate constant between compartment 2 and compartment 1; LogP: logarithm of partition coefficient between organic and aqueous solution; Peff: effective permeability; pKa: negative logarithm of the acid dissociation constant; T½: apparent terminal elimination half life; Vc: volume of central compartment;
^{a} Artursson P, Karlsson J (1991) Correlation between oral drug absorption in humans and apparent drug permeability coefficients in human intestinal epithelial (Caco-2) cells. Biochem Biophys Res Comm; 175: 880-5.

TABLE 5
Predicted branaplam exposures after single
administration of branaplam in adults

| Dose | Fa | Tmax | Cmax | AUC0-24 h | AUC0-168 h |
|---|---|---|---|---|---|
| (mg) | (%) | (h) | (ng/mL) | (h*ng/mL) | (h*ng/mL) |
| 15 | 96 | 3.7 | 9.74 | 108 | 367 |
| 35 | 96 | 3.7 | 23 | 253 | 856 |
| 50 | 96 | 3.7 | 32 | 361 | 1223 |
| 105 | 95 | 3.7 | 67 | 742 | 2529 |
| 200 | 88 | 3.6 | 122 | 1297 | 4506 |
| 210 | 88 | 3.6 | 127 | 1351 | 4701 |
| 400 | 80 | 3.5 | 234 | 2304 | 8173 |
| 420 | 80 | 3.5 | 245 | 2401 | 8522 |
| 630 | 75 | 3.5 | 356 | 3381 | 11997 |

AUC: area under the curve; Cmax: maximum concentration; Fa: fraction absorbed; Tmax: time of maximum concentration

### Example 1c.2: Pharmacodynamic/Pharmacokinetic Model Description

The pharmacodynamic/pharmacokinetic (PD/PK) model in adult subjects was developed using an adult PBPK model as described in Example 1c.1 and coupled with a PD model in GastroPlus™ software, Version 9.6 (SimulationPlus, Lancaster, CA, USA).

### Modeling Strategy

Establishment of PK/PD relationship in BacHD mouse model (see Example 1a) and development of a PK/PD model.
Development of PK/PD model in adult patients
Estimation of the anticipated efficacious dose range

### Model Development

For the establishment of the PK/PD relationship and the development of a mouse PK/PD model, the PK parameters in mouse plasma were estimated considering PK data from studies with male C57BL/6 mice (10 mg/kg, single dose), rasH2 mice (1, 3, 4 and 10 mg/kg, repeated daily doses), and BacHD mice (10 and 50 mg/kg, single dose; 12 and 24 mg/kg, repeated, three times a week doses). The PK parameter analysis of this data pool was executed by means of population PK model with extra-vascular administration, a lag time (Tlag), 2-compartments (V1: volume of compartment 1; V2 volume of compartment 2, Q: intercompartmental clearance; ka: first order rate of absorption; CL: clearance). The population PK model was described and executed by Monolix software (Version 2018R1, Lixoft).

For the development of the PK/PD relationship, the concentration of mutant HTT protein in the brain of BacHD mice and its changes after branaplam administration were used as PD biomarker (Example 1a). To increase the data base, all concentrations of the mutant HTT protein were pooled. The correlation between branaplam concentrations in plasma and the mutant HTT protein concentrations in the brain were investigated by means of a turnover model (described below) considering the inhibition of the production of mutant HTT protein in the brain. Since mutant HTT protein was determined in brain cortex and brain striatum, the PK/PD correlations were executed for both brain parts separately. The population PK/PD model enabled the description of the observed time delay between Cmax of branaplam in plasma (between 3 to 6 h post dose) and maximum decrease of mutant HTT protein in the brain (about 72 h post dose). For the execution of the population PK/PD model, the PK parameters (presented in Table 6) from the previously described population PK model were fixed and the PD parameters were calculated. The Monolix software (Version 2018R1, Lixoft) and its turnover model described in the library (pkpd/oral1_2cpt_SigmoidindirectModelinhibitionKin_TlagkaCIV1QV2R0koutimaxIC50gamma) was used to determine the PD parameters in the mouse. The PD part of the model can be described by the following equation:

mutant HTT protein change over time=kin*(1−I max*max(Cc,0)\hat{ }gamma/(max(Cc,0)\hat{ }gamma+IC50\hat{ }gamma))−kout*R
kin: synthesis rate of mutant HTT protein
kout: degradation rate of mutant HTT protein
Imax: maximum inhibition effect
IC50: half-maximum inhibitory concentration
gamma: sigmoidicity of the drug effect
max(Cc,0): branaplam plasma concentration
R: mutant HTT protein level at a given time with baseline R0 of 1 since only relative changes to the baseline were determined in the BacHD mouse model, Example 1a

It has to be noted, that the maximum inhibitory effect was set to 1 and the mutant HTT protein baseline, R0, was set to 1 since only relative changes to the baseline were determined in the BacHD mouse (Example 1a). Therefore, the ratio R0=Kin/Kout (mutant HTT protein synthesis rate/mutant HTT protein degradation rate) indicated that kin and kout are equal.

For the development of a PK/PD model in adults, the adult ACAT/PBPK model (Example 1c.1) was used to predict the concentration-time course of branaplam in plasma after oral branaplam administration. The PD parameters of the population PK/PD model in mouse were scaled for brain cortex and brain striatum from BacHD mouse to human according to following assumptions:
Mutant HTT baseline level in brain (R0) was kept equal to 1.
Drug's potency (IC50) was assumed to be the same in human as in BacHD mouse. The value was not corrected by the plasma protein binding since values in mouse and human were 0.741 and 0.8 in mouse and human, respectively.
Degradation rate or fractional turnover parameter of the mutant HTT protein synthesis (kout) was scaled from BacHD mouse to human considering an allometric scaling method based on the assumption that endogenous turnover of proteins, peptides and hormones can be scaled across different species and are related to energy turnover or metabolic rates (Gabrielsson J, Hjorth S, Quantitative Pharmacology: An Introduction to Integrative Pharmacokinetic-Pharmacodynamic Analysis. Swedish Pharmaceutical Press; 1 edition (May 7, 2012)). The exponent of −0.2 empirically used for scaling rate constants (Mahmood I, et al, 1996, Interspecies scaling: predicting clearance of drugs in humans: Three different approaches. Xenobiotica 26:887-895 and Mahmood I, 2005, Prediction of oral pharmacokinetic parameters in humans, in Interspecies Pharmacokinetic Scaling: Principles and Application of Allometric Scaling pp 144-167, Pine House Publishers, Rockville, MD) was used for the allometric scaling and the equation kout human=kout mouse*(body weight human/body weight mouse)\hat{ }−0.2 was used for the estimation.

For the estimation of the anticipated efficacious dose range, the adult ACAT/PBPK model (Example 1c.1) and the PD model in adults were coupled and simulations executed by means of GastroPlus™ software, Version 9.6 (PD Plus module, SimulationPlus, Lancaster, CA, USA). Several dose-levels with twice a week dosing and once a week dosing regimens were simulated in adults to predict corresponding PK/PD profiles (vs time) and PK/PD parameters (e.g. maximum concentration, Cmax, and area under the curve, AUC; mHTT decrease in brain). The simulations targeted an approximate 50% reduction in mutant HTT protein in the brain, which is believed to be necessary for clinically meaningful slowing of disease progression (Kaemmerer W F and Grondin R C, 2019, The effects of huntingtin-lowering: what do we know so far?, Degenerative Neurological and Neuromuscular Disease, 9, pp 3-17).

### Results—PK/PD Model Based on BacHD Mouse

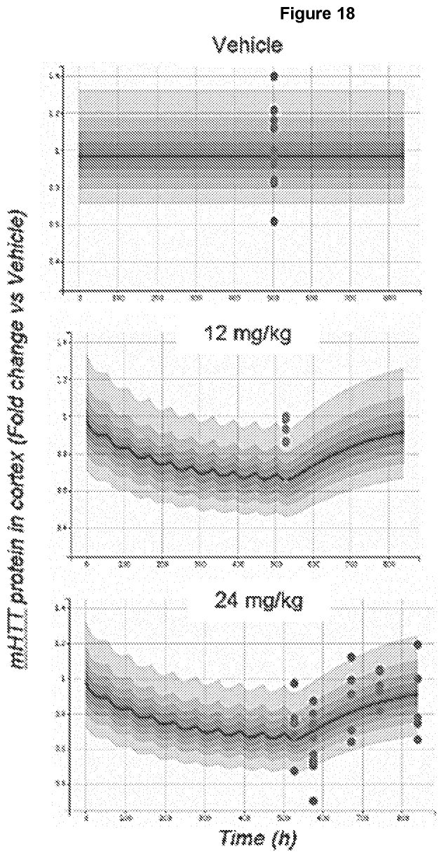

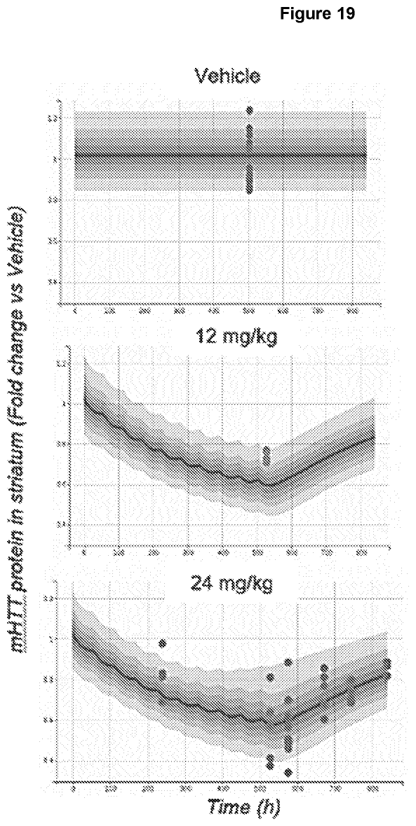

The parameter estimates of the PK/PD model are presented in Table 6. Predicted distribution of mutant HTT protein in the brain (cortex and striatum) of the BacHD mouse after triple oral administration of branaplam for 3 weeks are presented in FIG. 18 and FIG. 19.

TABLE 6
Parameter estimates of the mouse
PK/PD model based on BacHD mouse

| | Parameters | Estimate (RSE) | |
|---|---|---|---|
| popPK parameters (plasma) | Tlag (h) | 0.346 | (8.6%) |
| | ka (1/h) | 0.429 | (17.6%) |
| | CL/F (L/h/kg) | 3.35 | (3.59%) |
| | V1 (L/kg) | 28.2 | (7.25%) |
| | Q/F (L/h/kg) | 0.504 | (11.1%) |
| | V2 (L/kg) | 32.5 | (12.9%) |
| popPD parameters (cortex) | RO | 0.971 | (3.04%) |
| | kout (1/h) | 0.00634 | (8.16%) |
| | Imax | 1 | |
| | IC50 (ng/mL) | 174 | (8.55%) |
| popPD parameters (striatum) | RO | 1.02 | (3.39%) |
| | kout (1/h) | 0.00341 | (7.26%) |
| | Imax | 1 | |
| | IC50 (ng/mL) | 68.2 | (19.9%) |

CL: clearance of elimination from central compartment; F: bioavailability; IC50: half-maximum inhibitory concentration; Imax: maximum inhibition effect; ka: first-order absorption rate constant; kout: degradation rate of mutant HTT protein; Q: intercompartmental clearance; RO: baseline of mutant HTT protein concentrations in the brain; RSE: relative standard error reported on the approximate standard deviation scale (SE/variance)/2; Tlag: lag time of absorption; V1: central volume of 2-compartment PK model; V2: peripheral volume of 2-compartment PK model

### Results—PK/PD Model in Adult Patients

The parameter estimates of the adult ACAT/PBPK model are presented in Example 1c.1 and the scaled population PD data in Table 7.

TABLE 7
Parameter estimates of the human
PK/PD model based on BacHD mouse

| | | | Parameters | Estimate (RSE) |
|---|---|---|---|---|
| Scaled | PD | parameters | RO | 1 |
| (cortex) | | | kout (1/h) | 0.00130 ^{a} |
| | | | T½ (days) | 22 ^{b} |
| | | | Imax | 1 |
| | | | IC50 (ng/mL) | 0.174 ^{c} |
| Scaled | PD | parameters | RO | 1 |
| (cortex) | | | kout (1/h) | 0.000697 ^{a} |
| | | | T½ (days) | 48 ^{b} |
| | | | Imax | 1 |
| | | | IC50 (ng/mL) | 0.0682 ^{c} |

^{a} Allometric scaling of protein turnover based on body weight, kout man = kout mice × (BW man/BW mouse)\hat{ } −0.2;
^{b} mutant HTT protein T½ = Ln(2)/kout;
^{c} Assumes same IC50 in human as in BacHD mouse
IC50: concentration at 50% of Imax; Imax: maximum inhibition effect; kout: fractional turnover parameter of mutant HTT protein; RO: baseline of mutant HTT protein concentrations in the brain; T½: half life

### Results—Anticipated Efficacious Dose in Adult Patients

The developed PK/PD model in adult patients was used to simulate the plasma concentration-time courses of branaplam and the corresponding decrease of mutant HTT protein in the brain (cortex and striatum) following weekly or twice-weekly oral doses of branaplam. The simulations targeted a maximum of about 50% reduction in mutant HTT protein in the brain, which is believed to be necessary for clinically meaningful slowing of disease progression (Kaemmerer W F and Grondin R C, 2019, The effects of huntingtin-lowering: what do we know so far?, Degenerative Neurological and Neuromuscular Disease, 9, pp 3-17). The anticipated efficacious dose range of branaplam was predicted to range between 140 and 560 mg once a week and 70 and 280 mg twice a week. Higher doses are considered to result in a higher decrease of the mutant HTT protein in the brain with a potential higher benefit. However, the potential increase of the adverse events has to be balanced in a risk-benefit assessment.

The predicted exposure parameters and the predicted, corresponding decrease of mutant HTT protein in the brain for the identified dose range at steady state are presented in Table 8.

TABLE 8
Predicted PK and PD values in adult patients at steady state

| | | Cmax | Ctrough | AUCweek, | Predicted mHTT |
|---|---|---|---|---|---|
| Dose | Dose | ss | ss | ss | protein decrease |
| (mg) | regimen | (ng/mL) | (ng/mL) | (h*ng/mL) | in brain |
| 70 | BIW | 60 | 14 | 3960 | 12-25% |
| 140 | QW | 95 | 8 | | |
| 140 | BIW | 115 | 28 | 7430 | 20-40% |
| 280 | QW | 180 | 15 | | |
| 280 | BIW | 215 | 50 | 13540 | 30-55% |
| 560 | QW | 350 | 26 | | |

AUC: area under the curve; BIW: twice a week; Cmax: maximum concentration; Ctrough: minimum concentration; QW: once a week; ss: steady state

### Example 2: Clinical Evaluation of Branaplam

### Example 2.1

A two-part, placebo-controlled dose range finding study to evaluate the safety, tolerability, pharmacokinetics and pharmacodynamics of branaplam when administered as once weekly or twice weekly oral doses in subjects with Huntington's disease.

| Objective(s) | Endpoint(s) |
|---|---|
| Primary objective(s) | Endpoint(s) for primary objective(s) |
| To evaluate the safety and tolerability of | Adverse events, physical exam findings |
| branaplam when administered as once | (including neurological assessments) vital |
| weekly or twice weekly up to 52 weeks | signs, cardiovascular findings, safety |
| in patients with Huntington's disease | laboratory assessments including chemistry, |
| (HD) | hematology, and urinalysis, ophthalmologic |
| | evaluation, treatment-induced neuropathy |
| | assessment scale, testicular atrophy |
| | assessments, and Columbia-Suicide Severity |
| | Rating Scale (C-SSRS), |
| To evaluate the pharmacokinetics and | Plasma and cerebrospinal fluid (CSF) |
| pharmacodynamics of branaplam when | concentrations of branaplam and metabolites |
| administered as once weekly or twice | Plasma and CSF concentrations of mutant |
| weekly up to 52 weeks in patients with | huntingtin (mHTT) and total huntingtin (HTT) |
| Huntington's disease | |
| Secondary objective(s) | Endpoint(s) for secondary objective(s) |
| To assess the effect of branaplam when | Change from baseline compared to placebo |
| administered as once weekly or twice | (PBO) in: |
| weekly up to 52 weeks in patients with | Stroop Word Reading Test (SWRT) |
| Huntington's disease (HD) on clinical | Symbol Digit Modalities Test (SDMT) |
| endpoints relevant to Huntington's | Montreal Cognitive Assessment (MoCA) |
| disease | Unified Huntington's Disease Rating Scale Total |
| | Function Capacity (UHDRS-TFC) |
| | Unified Huntington's Disease Rating Scale Total |
| | Motor Score (UHDRS-TMS) |
| | Unified Huntington's Disease Rating Scale |
| | Independence Score (UHDRS-IS) |
| | Clinical Global Impression Severity Scale (CGI-S) |
| | Apathy Evaluation Scale (AES), self and physician |
| | forms |
| | Hospital Depression and Anxiety Scale (HADS) |
| | Ventricular, Caudate and Total Brain Volume as |
| | measured by structural magnetic resonance |
| | imaging |
| Exploratory objective(s) | Endpoint(s) for exploratory objective(s) |
| To explore the effects of branaplam | Neurofilament light chain (NFL) in serum and |
| when administered as once weekly or | CSF |
| twice weekly doses up to 52 weeks in | |
| patients with Huntington's disease (HD) | |
| on pharmacodynamic biomarkers | |
| relevant to Huntington's disease | |

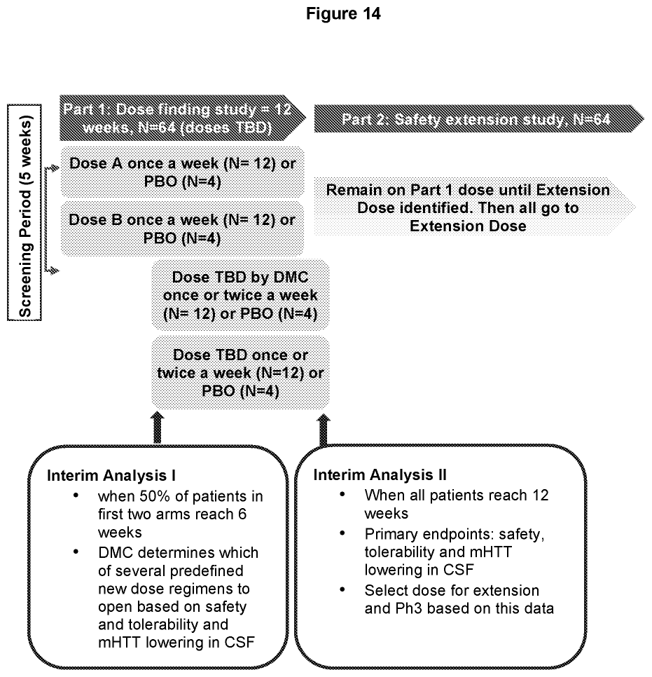

A total of 64 subjects are randomized to 1 of 4 treatment groups in an adaptive fashion (FIG. 14). Each treatment group is randomized active:placebo 3:1. The first 32 subjects are randomized to one of two treatment groups while the last 32 subjects are randomized to a dose regimen per Data Monitoring Committee (DMC) recommendations. A total of 2 interim analyses (IAs) are planned during Part 1 of the study. IA-1, conducted once 16 subjects have completed 6 weeks of treatment, to inform the dose selection for the last 2 treatment groups. IA-2, conducted once all subjects (64) have completed 12 weeks of treatment, to inform the dose selection for Part 2.

Both IAs are to be conducted by an external DMC.

### Part 1: Dose Range Finding

Following confirmation of eligibility, subjects complete a baseline assessment (˜over a day period), and a multi-dose treatment period (varying across subjects) at the assigned treatment regimen. Safety, tolerability, PK/PD and clinical endpoints relevant to HD are collected as outlined in the assessment schedule. The frequency of sample collection/study assessments is greater during the first 12 weeks of treatment due to the nature of the study. Following the confirmation of eligibility patients are randomized to receive either active or placebo treatment as an oral solution administered twice weekly. A total of 7 visits is required during the first 12 weeks. Some visits, if deemed appropriate, may be conducted at an at home basis by a qualified visit health care professional. Patients are presented with the opportunity to continue participation in the study at the 12-week visit. If a subject chooses to remain in the study they continue to receive the assigned study treatment and complete clinic visits on a monthly basis until the open label extension is activated. Subjects. If a subject does not wish to continue an End Of study Evaluation is completed.

### Part 2: Open Label Extension

Once the final subject (64) completes week 12 in Part 1, the DMC reviews all available data to make a final dose recommendation for Part 2. Sites are notified and subjects return to the study clinic to commence Part 2. Prior to the first open label dose a series of assessments are collected, as outlined in the assessment schedule. Clinic visits take place every 6 weeks until the subject reaches week 52 of study participation.

### Population

Approximately 62 male or female subjects with confirmed Stage I or II Huntington's Disease are enrolled in this study to allow for a completion of 60 subjects (following 12 weeks of treatment).

### Inclusion Criteria

Subjects eligible for inclusion in this study must meet all of the following criteria:
1. Written informed consent must be obtained before any assessment is performed.
2. Must be capable of providing informed consent (in the opinion of the Investigator)
3. Clinically diagnosed manifest Huntington's disease with a UHDRS Total Functional Capacity (TFC)>7 at screening
4. Genetically confirmed Huntington's disease, with presence of ≥ 36 CAG repeats (SEQ ID NO: 22) in the huntingtin gene
5. Male and female subjects between 25 to 75 years of age, inclusive, on the day of Informed Consent signature

### Exclusion Criteria

Subjects meeting any of the following criteria are not eligible for inclusion in this study.
1. Any medical history of brain or spinal disease what would interfere with the Lumbar Puncture process, CSF circulation or safety assessments.
2. Score “yes” on item 4 or item 5 of the Suicidal Ideation section of the C-SSRS, if this ideation occurred in the past 6 months, or “yes” on any item of the Suicidal Behavior section, except for the “Non-Suicidal Self-Injurious Behavior” (item also included in the Suicidal Behavior section), if this behavior occurred in the past 2 years.
3. Sexually active males must use a condom during intercourse while taking drug and for 6 months after stopping branaplam medication and should not father a child in this period. A condom is required to be used also by vasectomized men in order to prevent delivery of the drug via seminal fluid.
4. Use of other investigational drugs within 5 half-lives of enrollment, or within 30 days, whichever is longer.
5. History of hypersensitivity to any of the study drugs or its excipients or to drugs of similar chemical classes.
6. Cardiac or cardiac repolarization abnormality, including any of the following:
   History of myocardial infarction (MI), angina pectoris, or coronary artery bypass graft (CABG) within 6 months prior to starting study treatment.
   ·Clinically significant cardiac arrhythmias (e.g., ventricular tachycardia), complete left bundle branch block, high-grade AV block (e.g., bifascicular block, Mobitz type II and third degree AV block).
7. Resting QTcF≥450 msec (male) or ≥460 msec (female) at pretreatment [screening and baseline] or inability to determine the QTcF interval.
8. Subjects taking medications that are inhibitors of CYP3A4 (e.g., clarithromycin, conivaptan, indinavir, itroconazole, ketoconazole, ritonavir, mibefradil, nefazodone, nelfinavir, posaconazole, saquinavir, telaprevir, telithromycin, voriconazole, etc.).
9. History of malignancy of any organ system (other than localized basal cell carcinoma of the skin or in situ cervical cancer), treated or untreated, within the past 5 years, regardless of whether there is evidence of local recurrence or metastases.
10. Pregnant or nursing (lactating) women.
11. Women of childbearing potential, defined as all women physiologically capable of becoming pregnant, unless they are using highly effective methods of contraception during dosing and for 6 months) after stopping the study medication. Highly effective contraception methods include:
   Total abstinence (when this is in line with the preferred and usual lifestyle of the subject). Periodic abstinence (e.g., calendar, ovulation, symptothermal, post-ovulation methods) and withdrawal are not acceptable methods of contraception.
   Female sterilization (have had surgical bilateral oophorectomy with or without hysterectomy) total hysterectomy or tubal ligation at least six weeks before taking investigational drug. In case of oophorectomy alone, only when the reproductive status of the woman has been confirmed by follow up hormone level assessment.
   Male sterilization (at least 6 months prior to screening). For female subjects on the study, the vasectomized male partner should be the sole partner for that subject.
   Use of oral, (estrogen and progesterone), injected or implanted hormonal methods of contraception or placement of an intrauterine device (IUD) or intrauterine system (IUS) or other forms of hormonal contraception that have comparable efficacy (failure rate <1%), for example hormone vaginal ring or transdermal hormone contraception.

In case of use of oral contraception, women should have been stable on the same pill for a minimum of 3 months before taking investigational drug.

In case local regulations deviate from the contraception methods listed above, local regulations apply and are described in the informed consent form (ICF).

Women are considered post-menopausal and not of child bearing potential if they have had 12 months of natural (spontaneous) amenorrhea with an appropriate clinical profile (e.g. age appropriate, history of vasomotor symptoms) or have had surgical bilateral oophorectomy (with or without hysterectomy), total hysterectomy or tubal ligation at least six weeks ago. In the case of oophorectomy alone, only when the reproductive status of the woman has been confirmed by follow up hormone level assessment is she considered not of childbearing potential.
12. Any medical history or condition that would interfere with the ability to complete the protocol specified assessments. e.g., implanted shunt, conditions precluding MRI scans etc.
13. At significant risk of suicide, major depressive episode, psychosis, confusional state or violent behavior as assessed by the Investigator.
15. Antidepressants or benzodiazepine use unless stable dose for at least 12 weeks prior to Screening and with a dose regimen that is not anticipated to change during the study.
16. Active infection requiring systemic antiviral or antimicrobial therapy that will not be completed at least 3 days prior to first study drug administration (Day 1).
17. Any history of gene therapy or cell transplantation or any other experimental brain surgery.
18. Subjects who are not capable of giving consent, persons depending on the sponsor, investigator or site as well as persons who have been committed to an institution by way of official or judicial order.
19. History of hepatitis B or hepatitis C or serologic evidence for active viral hepatitis (HBsAg and HCVab test).
20. Any surgical or medical condition which might put the subject at risk in case of participation in the study. The Investigator should make this determination in consideration of the subject's medical history and/or clinical or laboratory evidence of any of the following:
   Lipase and/or amylase must not exceed the 1.5× upper limit of normal (ULN)
   Liver disease or liver injury as indicated by abnormal liver function tests. ALT (SGPT), AST (SGOT), γ-GT, alkaline phosphatase and serum bilirubin will be tested.
   Any of the following single parameters in serum of ALT, AST, γ-GT, alkaline phosphatase or bilirubin must not exceed 1.5× upper limit of normal (ULN).
   Any elevation above ULN of more than one parameter of ALT, AST, γ-GT, alkaline phosphatase or serum bilirubin will exclude a subject from participation in the study.
   History of renal injury/renal disease or presence of impaired renal function as indicated by any elevation above ULN of creatinine or BUN and/or urea values, or the presence of abnormal urinary constituents (e.g., albuminuria).
   Evidence of urinary obstruction or difficulty in voiding at screening.
21. History of immunodeficiency diseases, including a positive HIV (ELISA and Western blot) test result.
22. History of drug or alcohol abuse or evidence of such abuse as indicated by the laboratory assays conducted during screening.
23. Significant illness which has not resolved within two (2) weeks prior to initial dosing.
25. Stable medical, psychiatric and neurological status for at least 12 weeks prior to screening and at the time of enrollment.
26. Not able or willing to complete all assessments in protocol.
27. Clinically significant signs of testicular abnormalities via ultrasound assessments.
28. Clinically significant retinal abnormalities.

### Example 2.2: Evaluation of the Effect of Branaplam on the Expression Levels of Huntingtin (HTT) mRNA in Infants with Type I Spinal Muscular Atrophy

### Methods

### Open-Label Multi-Part First-In-Human Proof of Concept Study of Oral Branaplam

The effect of branaplam on the expression levels of Huntingtin (HTT) mRNA was assessed in infants with Type I spinal muscular atrophy who were enrolled in an open-label multi-part first-in-human proof of concept study of oral branaplam.

The aim of part one of this study was to determine the safety and tolerability of ascending weekly doses and to estimate the maximum tolerated dose (MTD) of oral/enteral branaplam (see Example 3) in infants with Type 1 SMA. All patients had exactly 2 copies of the SMN2 gene, as determined e.g. by quantitative real time PCR or droplet digital PCR.

Patients were dosed once weekly with branaplam. The branaplam doses were escalated in subsequent cohorts until MTD was determined or when PK results confirmed that the MTD could not be reached due to a potential pharmacokinetic exposure plateau at higher doses.

The starting dose was 6 mg/m² (approximately 0.3125 mg/kg). Subsequent doses were 12 mg/m², 24 mg/m², 48 mg/m² and 60 mg/m² (approximately 0.625 mg/kg, 1.25 mg/kg, 2.5 mg/kg and 3.125 mg/kg, respectively). Each cohort had 2-3 patients. All doses are of branaplam (free form). 14 patients were enrolled in Part 1; 13 patients were exposed to branaplam. The duration of exposure ranged from 4-33 months, 7 patients remain in the study. Six of the 7 patients are receiving 60 mg/m², 1 patient is receiving 48 mg/m². No dose-limiting toxicity was observed.

The aim of part two of this study is to evaluate the long-term safety and tolerability of 2 doses of branaplam administered weekly for 52 weeks in patients with Type 1 SMA. Part 2 of the study enrolls patients into 2 cohorts: cohort 1 at a 0.625 mg/kg dose and cohort 2 at a 2.5 mg/kg dose. The selected dose levels of 0.625 mg/kg and 2.5 mg/kg are based on all safety data from Part 1, as well as, all data from chronic juvenile toxicity studies available at the time of initiation of Part 2. Approximately 10 patients were planned to be enrolled in cohort 1 and 2. A total of twenty-five patients were enrolled and all received the treatment at least once, to date, 22 patients are still being treated for 6 to 18 months.

### Blood Collection

Whole blood samples from patients enrolled in part 1 and part 2 of the study were collected at baseline prior to treatment and at several time-points during treatment with branaplam (day 85 and then every 91 days). Parents of all examined participants provided written informed consent for additional biological research.

A 0.6 mL blood sample was collected with one Multivette® 600 Potassium EDTA (Sarstedt). After gentle mixing, the blood was transferred directly into the solution of a PAXgene Blood RNA tube (Becton Dickinson). The sample was immediately gently inverted 8 to 10 times to prevent clotting and left at room temperature in an upright position for 2 to 3 hours. After incubation, the PAXgene Blood RNA Tubes were stored at −20° C.

### RNA Extraction and Quantitative PCR

Total RNA was extracted using the PAXgene Blood RNA Kit (Qiagen). Total RNA was reverse transcribed to cDNA using random hexamers and the iScript™ Advanced cDNA Synthesis Kit (Bio-Rad). cDNA synthesis was performed according to manufacturer's instructions using 100 ng of total RNA as input into a 20 ul cDNA reaction to generate an initial cDNA with a concentration of 5 ng/ul (total RNA equivalents). Finally, the cDNA was subsequently diluted 1/1 with nuclease-free water to generate a final cDNA with a concentration of 2.5 ng/μl (total RNA equivalents). All preparations were carried out on ice. cDNA synthesis was performed on a C1000 Thermal cycler, Reaction Module 96W Fast (Bio-Rad) using the following conditions: 25° C. for 5 min, 46° C. for 20 min, 95° C. for 1 min and hold at 4° C. cDNA samples were stored at −20° C.

Levels of HTT mRNA and novel-exon-included HTT mRNA were then quantified by polymerase chain reaction (PCR) using the Bio-Rad QX200 droplet digital PCR system. Standard reaction and cycling conditions (95° ° C. for 10 min; 40 cycles of 94° C. for 30 sec and 60° C. for 60 sec; and 98° C. for 10 min; hold at 4° C.) and a cDNA input (total RNA equivalent) of 20 ng were applied.

For HTT mRNA levels, two independent predesigned quantitative PCR assays (Assay Hs.PT.58.14833829 with forward primer 5′-GAGACTCATCCAGTACCATCAG-3′ (SEQ ID NO: 10), reverse primer 5′-GATGTCAGCTATCTGTCGAGAC-3′ (SEQ ID NO: 11) and probe 5′-56-FAM/CGCTTCCAC/ZEN/TTGTCTTCATTCTCCTTGT/3IABKFQ-3′ (SEQ ID NO: 12) and assay Hs.PT.58.25550542 with forward primer 5′-GTAGAACTTCAGACCCTAATCCTG-3′ (SEQ ID NO: 13), reverse primer 5′-CACCACTCTGGCTTCACAA-3′ (SEQ ID NO: 14) and probe 5′-56-FAM/CCCGACAGC/ZEN/GAGTCAGTGATTGTT/3IABKFQ-3′ (SEQ ID NO: 15), purchased from Integrated DNA Technologies, Inc.) were used. A customized quantitative PCR assay with forward primer 5′-TCCTGAGAAAGAGAAGGACATTG-3′ (SEQ ID NO: 3), reverse primer 5′-CTGTGGGCTCCTGTAGAAATC-3′ (SEQ ID NO: 4) and probe 5′-56-FAM/AATTCGTGG/ZEN/TGGCAACCCTTGAGA/3IABKFQ-3′ (SEQ ID NO: 7) was applied to quantify the inclusion of a novel exon into HTT mRNA.

All gene expression values were normalized to Glucuronidase beta (GUSB) mRNA levels. A predesigned quantitative PCR assay (Assay Hs.PT.39a.22214857 with forward primer 5′-TCACTGAAGAGTACCAGAAAAGTC-3′ (SEQ ID NO: 16, reverse primer 5′-TTTTATTCCCCAGCACTCTCG-3′ (SEQ ID NO: 17) and probe 5′-HEX/ACGCAGAAA/ZEN/ATACGTGGTTGGAGAGC/3IABKFQ-3′ (SEQ ID NO: 18), purchased from Integrated DNA Technologies, Inc.) was used to assess GUSB mRNA levels.

### CONCLUSIONS

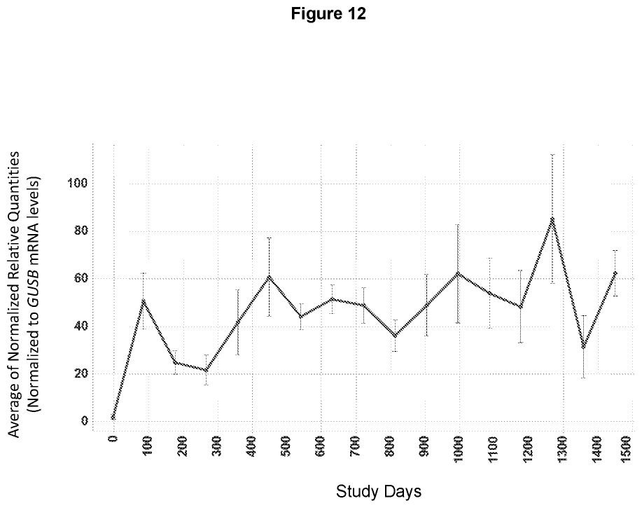

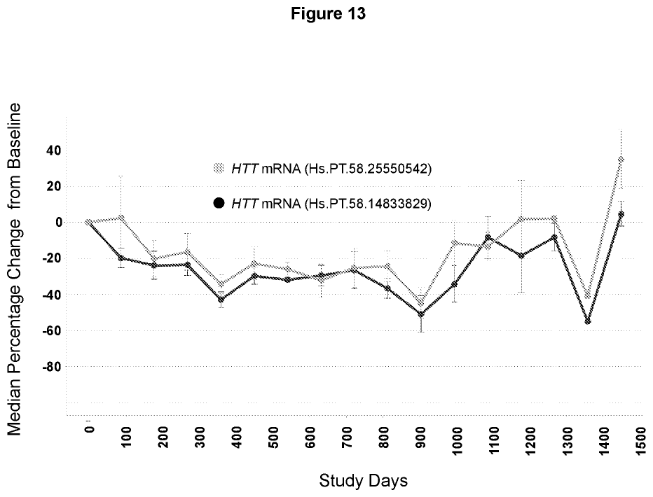

The effect of branaplam on the expression levels of Huntingtin (HTT) mRNA was assessed in infants with Type I spinal muscular atrophy who were enrolled in an open-label multi-part first-in-human proof of concept study of oral branaplam. Patients were dosed once weekly with branaplam. The longitudinal gene expression analysis of blood samples showed that the inclusion of the novel exon into HTT mRNA was induced after first weekly doses of branaplam and was kept sustained at constant levels over a period of 1450 study days (FIG. 12). In addition, blood HTT mRNA levels decreased by up to 50% from baseline over a period of 904 study days (FIG. 13). Afterwards, as assessed from only 1-5 long-term treated subjects depending on their progress within the clinical study, HTT mRNA levels returned to values around baseline levels between study days 904 and 1450 (FIG. 13). Our results demonstrate that branaplam treatment of infants with Type I spinal muscular atrophy induces the inclusion of a novel exon into blood HTT mRNA and lowers blood HTT mRNA levels by up to 50% as compared to baseline. These results demonstrate that sustained lowering of HTT to target therapeutic levels can be attained via intermittent dosing of branaplam.

FIG. 12: Weekly oral doses of branaplam induced and elevated blood HTT transcript levels with inclusion of a novel exon in infants with SMA Type 1. Longitudinal data from study days 358 to 1450 were available from only 1-5 subjects depending on progress of the individual subjects within the study. Error bars represent standard error.

FIG. 13: Weekly oral doses of branaplam lower blood HTT transcript levels in infants with SMA Type 1. Longitudinal data from study days 358 to 1450 were available from only 1-5 subjects depending on progress of the individual subjects within the study. Error bars represent standard error.

### Example 3: Oral Formulation of Branaplam

### Procedure

The required amount of 2-hydroxypropyl-beta-cyclodextrin was dissolved in 80% volume of target water (i.e. final intended volume) and stirred for 30 minutes. The required amount of branaplam monohydrochloride salt was then added to said solution, under stirring, at room temperature. The solution was stirred for 45 minutes after the addition was completed or for longer until a particle-free (i.e. to naked eye) solution was obtained. Initial pH adjustment was performed using NaOH 0.1M or HCl 0.1M to reach the intended pH (±0.25). The required volume of water was added to the solution to reach the final intended volume and stirred for at least 10 minutes at 25±3° C. after the addition was completed. Final pH adjustment was performed using NaOH 0.1M or HCL 0.1M to reach the intended pH.

| Ingredients | Amount |
|---|---|
| Branaplam monohydrochloride salt | 3.826 mg/ml {*} |
| 2-hydroxypropyl-beta-cyclodextrin | 17.5 percent (w/v) |
| Hydrochloride acid | q.s. to pH 4 |
| Sodium hydroxide | |
| Water | q.s. |
| pH adjusted to | 4 |

{*} Salt/base ratio on anhydrous basis 1.093

1. A method of treatment for slowing progression of Huntington's disease in a subject in need thereof, the method comprising administering to the subject a therapeutically effective amount of a compound selected from the group consisting of: 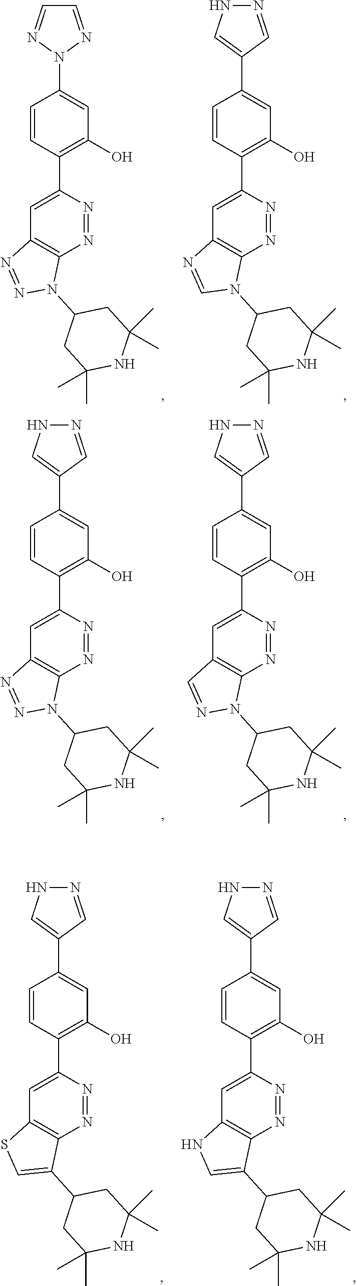 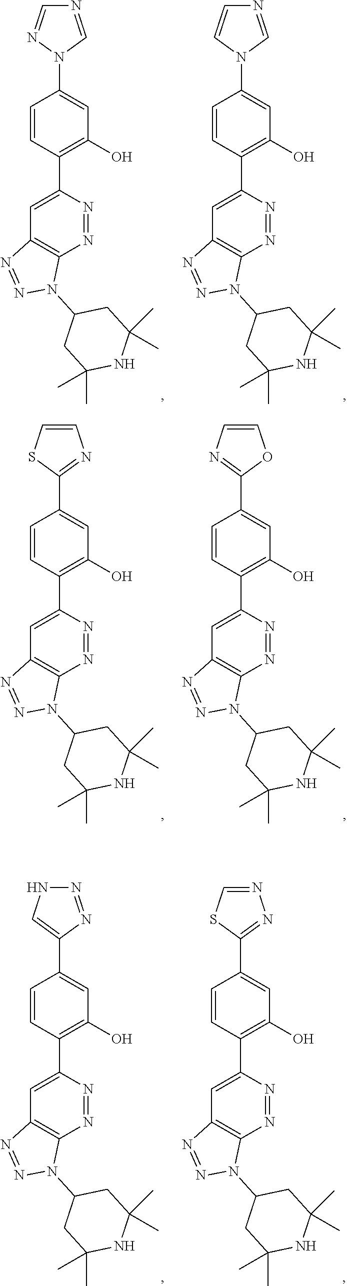 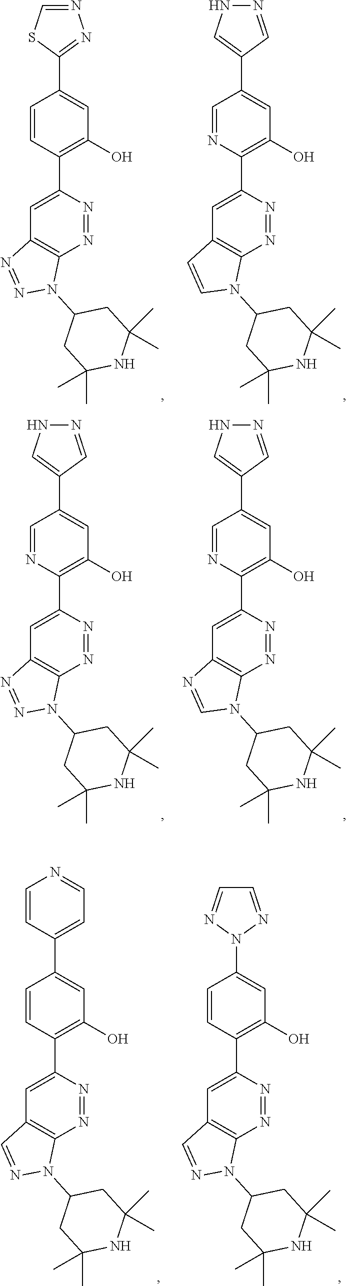 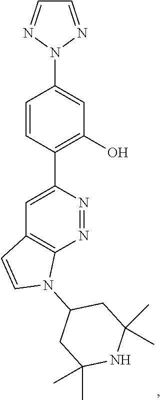 [SMILES: CC1(C)CC(n2ccc3cc(-c4ccc(-n5nccn5)cc4O)nnc32)CC(C)(C)N1]
and pharmaceutically acceptable salts thereof.

2. A method of treatment for Huntington's disease as a disease-modifying therapy, the method comprising administering to the subject a therapeutically effective amount of a compound selected from the group consisting of: 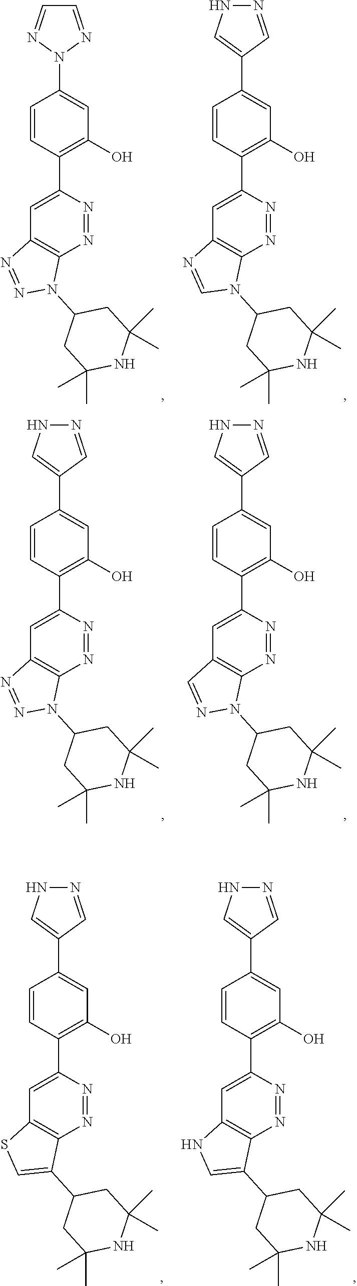 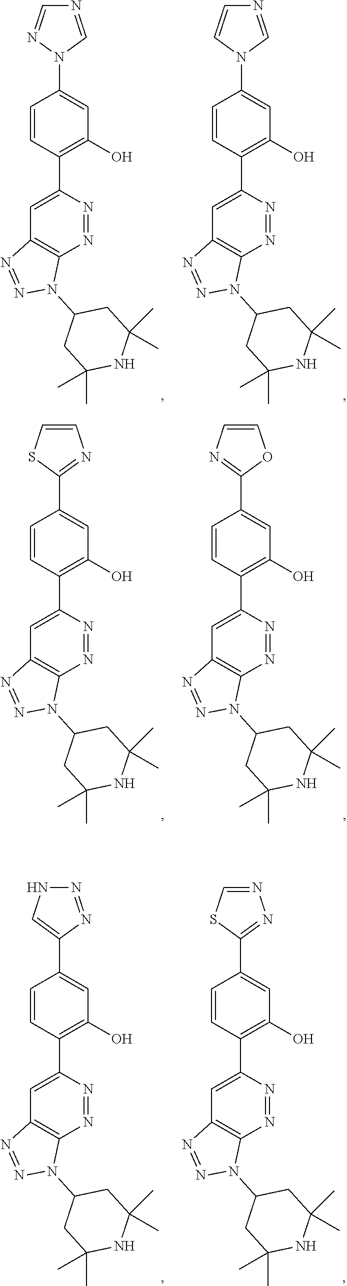 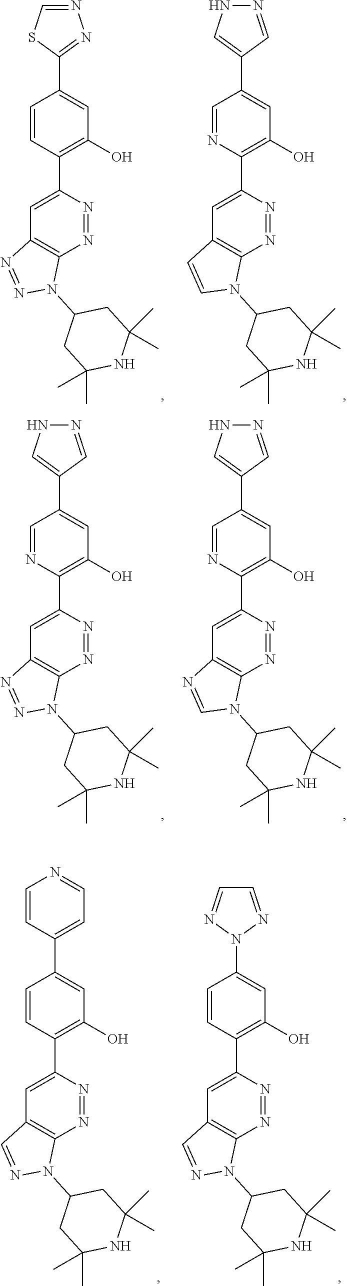 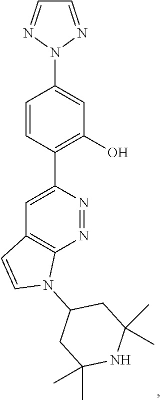 [SMILES: CC1(C)CC(n2ccc3cc(-c4ccc(-n5nccn5)cc4O)nnc32)CC(C)(C)N1]
and pharmaceutically acceptable salts thereof.

3. A method of treatment for slowing the decline of motor function associated with Huntington's disease, the method comprising administering to the subject a therapeutically effective amount of a compound selected from the group consisting of: 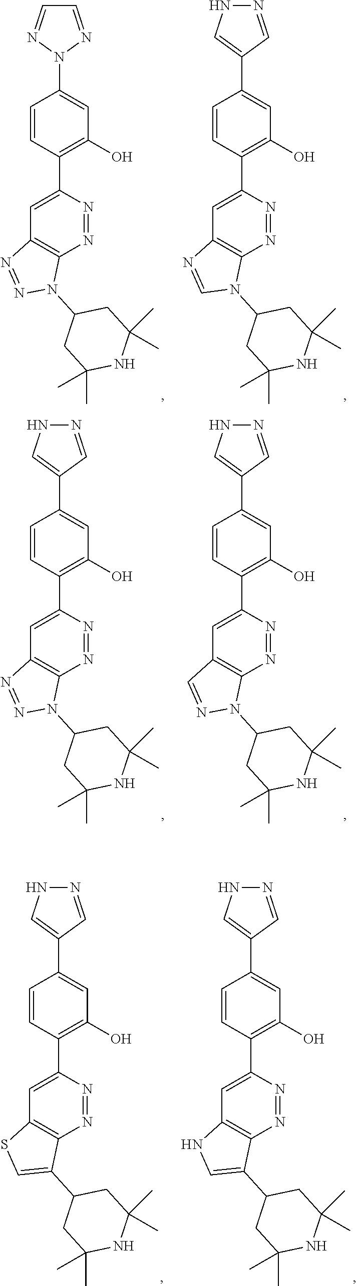 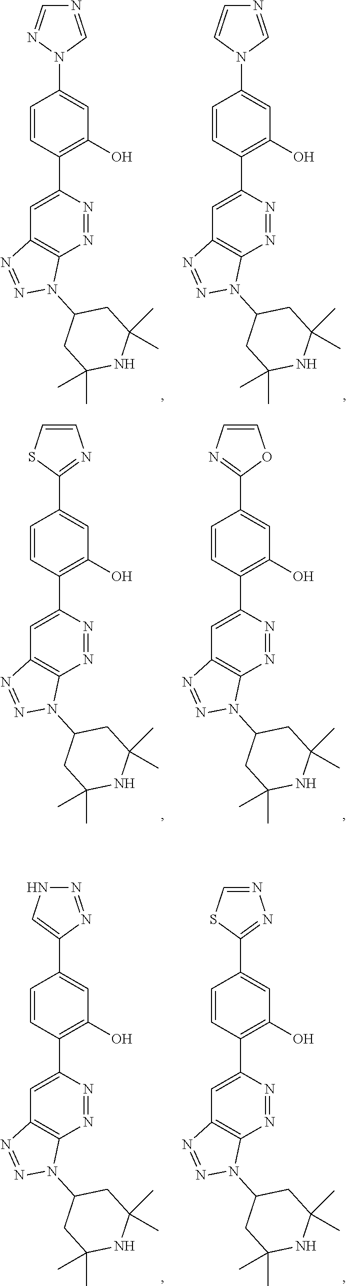 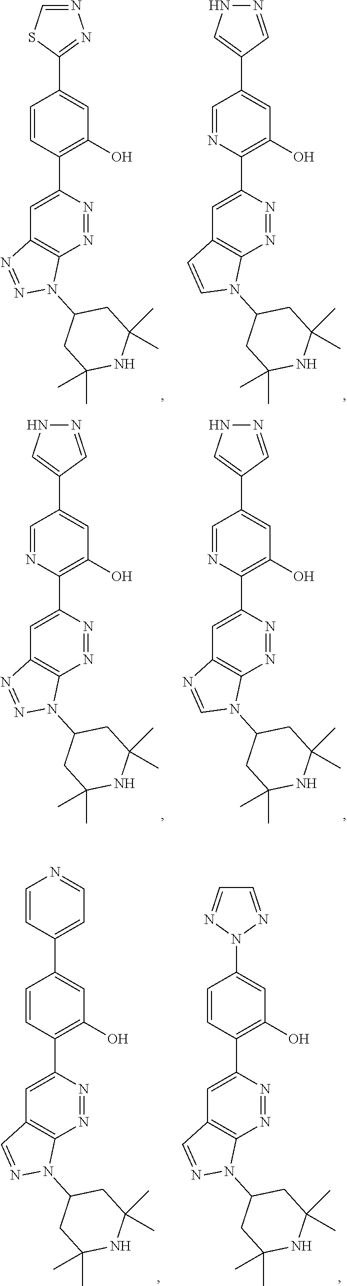 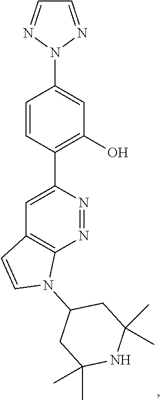 [SMILES: CC1(C)CC(n2ccc3cc(-c4ccc(-n5nccn5)cc4O)nnc32)CC(C)(C)N1]
and pharmaceutically acceptable salts thereof.

4.-7. (canceled)

8. The method of claim 3, wherein motor function comprises one or more selected from the group consisting of ocular motor function, dysarthria, dystonia, chorea, postural stability, and gait.

9.-11. (canceled)

12. The method of claim 1, wherein Huntington's disease is genetically characterized by CAG repeat expansion of from 36 to 39 (SEQ ID NO: 20) in the huntingtin gene on chromosome 4.

13. The method of claim 1, wherein Huntington's disease is genetically characterized by CAG repeat expansion >39 (SEQ ID NO: 21) in the huntingtin gene on chromosome 4.

14. The method of claim 1, wherein Huntington's disease is manifest Huntington's disease.

15. The method of claim 1, wherein Huntington's disease is juvenile Huntington's disease or pediatric Huntington's disease.

16. The method of claim 1, wherein Huntington's disease is in the early stage of Huntington's disease, middle stage of Huntington's disease, or advanced stage of Huntington's disease.

17. The method of claim 1, wherein Huntington's disease is stage I of Huntington's disease, stage II of Huntington's disease, stage III of Huntington's disease, stage IV of Huntington's disease, or stage V of Huntington's disease.

18. The method of claim 1, wherein Huntington's disease is pre-manifest Huntington's disease.

19. The method of claim 1, wherein the compound or a pharmaceutically acceptable salt thereof, is administered according to an intermittent dosing schedule.

20. The method of claim 1, wherein the compound or a pharmaceutically acceptable salt thereof, is administered once a week or twice a week.

21. (canceled)

22. The method of claim 1, wherein the compound or a pharmaceutically acceptable salt thereof, is administered in the form of a pharmaceutical composition.

23. The method of claim 1, wherein the compound or a pharmaceutically acceptable salt thereof, is administered in the form of a pharmaceutical combination.

24. The method of claim 1, wherein the compound or a pharmaceutically acceptable salt thereof, is administered following gene therapy or treatment with an antisense compound.

25. (canceled)

26. The method of claim 16, wherein Huntington's disease is in the early stage of Huntington's disease.

27. The method of claim 17, wherein Huntington's disease is stage I of Huntington's disease or stage II of Huntington's disease.

28. The method of claim 1, wherein the compound is: 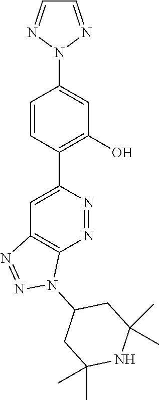 [SMILES: CC1(C)CC(n2nnc3cc(-c4ccc(-n5nccn5)cc4O)nnc32)CC(C)(C)N1]
or a pharmaceutically acceptable salt thereof.

29. The method of claim 2, wherein the compound is: 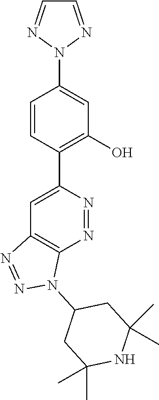 [SMILES: CC1(C)CC(n2nnc3cc(-c4ccc(-n5nccn5)cc4O)nnc32)CC(C)(C)N1]
or a pharmaceutically acceptable salt thereof.

30. The method of claim 3, wherein the compound is: 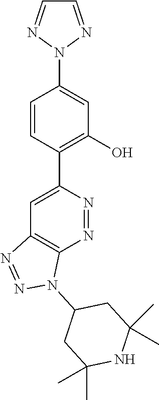 [SMILES: CC1(C)CC(n2nnc3cc(-c4ccc(-n5nccn5)cc4O)nnc32)CC(C)(C)N1]
or a pharmaceutically acceptable salt thereof.
